(19) United States
(12) Patent Application Publication
(10) Pub. No.: US 2009/0264628 A1
(43) Pub. Date: Oct. 22, 2009

(54) CRYSTAL STRUCTURE OF HUMAN UROKINASE PLASMINOGEN ACTIVATOR AMINO TEMINAL FRAGMENT BOUND TO ITS RECEPTOR

(75) Inventors: Mingdong Huang, Newton, MA (US); Qing Huai, Medford, MA (US); Graham C. Parry, San Mateo, CA (US); Douglas B. Cines, Wynnewood, PA (US); Andrew P. Mazar, Highland Park, IL (US)

Correspondence Address: BROWDY AND NEIMARK, P.L.L.C.;624 NINTH STREET, NW, SUITE 300, WASHINGTON, DC, 20001-5303, US

(73) Assignees: Beth-Israel Deaconess Medical Center , Inc., Boston, MA (US); Attenuon LLC, San Diego, CA (US); The University of Pennsylvania, Philadelphia, PA (US)

(21) Appl. No.: 12/089,166

(22) PCT Filed: Oct. 3, 2006
(86) PCT No.: PCT/US2006/038608

§ 371 Date: Nov. 26, 2008

Related U.S. Application Data
(60) Provisional application No. 60/722,402, filed on Oct. 3, 2005.

(51) Int. Cl. C07K 16/00; G06G 7/48

(52) U.S. Cl. 530/391.1; 703/11

(57) ABSTRACT

Urokinase-type plasminogen activator (uPA) binds its cellular receptor (uPAR) with high affinity, thus localizing the generation of plasmin from plasminogen on the surface of a variety of cells. Disclosed herein is the structure of suPAR (uPAR¹⁻²⁷⁷) complexed with the amino terminal fragment (ATF) of uPA (uPA¹⁻¹⁴³) at a resolution of 1.9 ú by X-ray crystallography. Three consecutive domains of uPAR (D1, D2 and D3) form the shape of a thick-walled teacup with a cone shape cavity in the middle, which has a wide opening (25 ú) and large depth (14 ú). uPA¹⁻¹⁴³ inserts into the cavity of uPAR and forms a large interface. The structure provides the basis for high affinity binding between uPA and uPAR and suggests the D1 and D2 domain of uPAR and the GFD domain of uPA (uPA⁷⁻⁴³) are primarily responsible for uPA-uPAR binding. This structure presents the first high resolution view of uPA-uPAR interaction, and provides, among other things, a new platform for designing uPA-uPAR inhibitors/antagonists.

### STATEMENT OF RIGHTS TO INVENTIONS MADE UNDER FEDERALLY SPONSORED RESEARCH

This invention was funded in part by a grant RO1 HL60169 from the National Heart Lung and Blood Institute of the National Institutes of Health (to co-inventor D. Cines) which provides to the United States government certain rights in this invention.

### BACKGROUND OF THE INVENTION

1. Field of the Invention

The invention in the field of structural biology and biochemistry relates to a novel 3D structure determined by x-ray crystallography of a ternary complex of the amino terminal fragment (ATF) of the urokinase-type plasminogen activator (uPA) together with a soluble form of its cell surface receptor (suPAR) and an antibody that binds to suPAR without disrupting ATF-suPAR binding, as well as uses of this structural information to design or screen putative inhibitors of ATF-suPAR interactions. The invention also relates to novel methodologies to generating binary, ternary or quartenary complexes of suPAR, ATF-suPAR, uPA-suPAR with ligands such as an antibody against suPAR or against uPA, or other ligands for suPAR such as integrins and vitronectin for the purpose of generating crystals that diffract to high resolution and are therefore expected to yield high resolution structures suitable for drug discovery and structure based drug design.

2. Description of the Background Art

Urokinase-type plasminogen activator (uPA) together with its cell surface receptor (uPAR) mediate surface-bound plasminogen activation (Myohanen, H et al. (2004) Cell Mol Life Sci 61:2840-58), and have been recognized to play important roles in a variety of cellular functions, including cell adhesion, migration, tissue remodeling, and tumor invasion (Andreasen, P A et al. (2000) Cell Mol Life Sci 57:25-40; Blasi, F et al. (2002) Nat Rev Mol Cell Biol 3:932-43; Ploug, M (2003) Curr Pharm Des 9:1499-528; Mondino, A et al. (2004) Trends Immunol 25:450-5). The molecular basis of these broad physiological roles comes from uPAR's capability to interact with many ligands, e.g., uPA, vitronectin, β1-, β2- and β3-integrins, G-protein coupled receptors, etc. Knowledge of the three-dimensional (3D) structure of uPA/uPAR complexes will provide crucial insights into the molecular mechanisms responsible for many of the unique properties of the uPA-uPAR interaction.

uPA is made up of a serine protease domain located at its carboxy-terminus (C-terminus) and a modular amino-terminal (N-terminal) fragment “ATF”, amino acid residues 1-135 (also referred to herein as uPA¹⁻¹⁴³) that includes a growth factor-like domain (GFD) and a kringle domain (KrD). uPA¹⁻¹⁴³ of uPA is responsible for the receptor binding, forming a stable complex with a dissociation constant of 0.28 nM (Ploug, M et al. (1994) FEBS Lett 349:163-8). uPAR is a 313-amino acid glycoprotein linked to the cell surface through a C-terminal glycosyl phosphatidylinositol (GPI) anchor (Ploug et al., supra). Soluble uPAR variants (suPAR) consisting of residues uPAR¹⁻²⁷⁷ without the GPI anchor have been identified under physiologic and pathological conditions, such as in patients with malignancies (Pappot, H et al (1997) Eur J Cancer 33:867-72) or paroxysmal nocturnal hemoglobinuria (PNH) (Ronne, E et al (1995) Br J Haematol 89:576-81; Gao, W et al (2002) Int J Hematol 75:434-9). suPAR binds uPA with a K_{d} in the subnanomolar range that is indistinguishable from the GPI-anchored full-length uPAR (Ploug et al., supra), indicating that suPAR is an appropriate candidate for the structural study of uPA-uPAR interactions in vitro.

### ABBREVIATIONS

uPA: Urokinase-type plasminogen activator
uPAR Urokinase-type plasminogen activator receptor
suPAR soluble uPAR, residues 1-277 (without the GPI anchor), also referred to herein as uPAR¹⁻²⁷⁷. uPAR and suPAR are used interchangeably when discussing the complexes subjected to x-ray crystallography and 3D structures discovered.
ATF amino-terminal fragment of uPA which may be residues 1-135 or 143 of uPA. In the present invention, uPA¹⁻¹⁴³ was used and this term is used interchangeably with “ATF.”
GFD Epidermal growth factor-like domain of uPA (included in ATF), residues 7-43 of uPA, also referred to herein as uPA⁷⁻⁴³
KrD kringle domain of uPA, included in ATF, residues 50-135 or 50-143, also referred to herein as uPA⁵⁰⁻¹⁴³
GPI glycosylphosphatidylinositol
mAb monoclonal antibody
ATN-615 a mAb raised against suPAR. The Fab fragment of the mAb was used in the present invention, and this fragment may also be referred to herein as ATN-615 (see PCT/US2005/18322, published as WO2005/116077).
rmsd root mean square deviation

### SUMMARY OF THE INVENTION

The present inventors disclose the crystal structure at a 1.9 Å resolution of suPAR-uPA¹⁻¹⁴³ further complexed with a Fab fragment of mAb, ATN-615, that was raised against suPAR. The ternary complex is referred to as “uPAR-uPA¹⁻¹⁴³-Fab” or as “uPAR/uPA¹¹⁴³/Fab”.

Based on knowledge from this structure, the present invention permits design and/or testing of more universal antagonists of uPA-uPAR interactions not limited by species specificities. This derives from the finding that amino acid residues involved in the first region of the uPAR-uPA interface are highly conserved among different species, so that an antagonist that targets this region inhibits human and mouse (and rat, etc.) uPAR-uPA interactions.

The present invention provides a platform for rational design of inhibitors of uPAR-uPA interactions that would be expected to prevent, reverse or attenuate the pathophysiological consequences of these interactions. One example of such consequences is tumor metastasis.

The present invention also relates to methods for forming crystals that diffract to high resolution. In the absence of an antibody that binds to the ATF-suPAR complex, crystals of ATF-suPAR diffract much more poorly (to 3.1 Å). The present invention describes the use of ligands for uPA or uPAR including antibodies, peptides, other proteins and small molecules that, when bound, allow the formation of crystals that diffract to high resolution.

In a preferred embodiment, the present invention is directed to a composition comprising a crystallized complex of uPA or a fragment thereof bound to a soluble uPAR molecule and further bound to, and constrained by, a ligand that has an affinity for uPAR of at least about 100 μM, preferably at least about 1 μM, more preferably at least about 10 nm, and which binds uPAR without disrupting uPA-uPAR binding interactions.

In the above composition, the uPA is preferably human uPA and the uPAR is preferably human uPAR and the ligand is preferably a uPAR-specific antibody or antigen-binding fragment thereof. Alternatively, the ligand may be a uPA-specific antibody or an antigen-binding fragment thereof. A preferred antibody is the anti-uPAR mAb designated ATN-615.

The above composition preferably is characterized by having a 3D atomic structure of the complex defined by a set of structural coordinates corresponding to the set of structural coordinates set forth in Table 1 and FIG. 4 (which show the x-ray crystallographic details and coordinates). In one embodiment, the complex is defined by a set of structural coordinates having a root mean square deviation (rmsd) of not more than about 1.2 Å, preferably not more than about 0.6 Å, most preferably not more than about 0.3 Å from the set of structure coordinates set forth in Table 1 and FIG. 4 (showing the x-ray crystallographic details and coordinates).

Also provided is a computing platform for generating a 3D model of a uPA-uPAR complex further constrained by a uPAR ligand, which computing platform comprises:
(a) a data-storage device storing data comprising a set of structural coordinates defining the structure of at least a portion of a 3D crystal structure of the uPA-uPAR complex; and
(b) a data processing unit for generating the 3D model from the data stored in the data-storage device.

A computer generated model of the present invention preferably represents the conformationally constrained 3D structure of a uPA-uPAR complex to which is also bound a ligand for uPAR, the computer generated model having a 3D atomic structure defined by a set of x-ray crystallographic coordinates set out in Table 1 and FIG. 4, or a set of structure coordinates defining at least a portion of a structure defined by the above x-ray crystallographic coordinates.

The invention includes a computer readable medium comprising, in a retrievable format, data that include a set of structure coordinates defining at least a portion of a 3D crystallographic structure of a crystallized uPA-uPAR complex that is conformationally constrained by being bound to a ligand for uPAR. A preferred ligand above is an Fab fragment of mAb ATN-615

In the above computer readable medium, the structure coordinates defining at least a portion of a 3D structure of the crystallized complex correspond to a set of coordinates set forth in Table 1 and FIG. 4, or have rmsd values of not more than about 1.2 Å, preferably not more than about 0.6 Å, most preferably not more than about 0.3 Å from the set of structure coordinates set forth in Table 1 and FIG. 4.

This invention includes a method of crystallizing a ternary complex of uPA, soluble uPAR and a ligand for uPAR:
(a) forming a binary suPAR-uPA complex by incubating uPA with suPAR at a molar ratio of about 1:1 at room temperature in a first buffer, and purifying the complex;
(b) mixing the binary complex at about a 1:1 molar ratio in a second buffer with a ligand that binds suPAR when the suPAR is bound to uPA, and purifying and concentrating a ternary 1:1:1 suPAR-uPA-ligand complex; and
(c) subjecting the ternary complex to microdialysis with a crystallization buffer, thereby crystallizing the ternary complex.

### BRIEF DESCRIPTION OF THE DRAWINGS

FIG. 1A is a stereo view of the overall structure of suPAR-uPA¹⁻¹⁴³-ATN-615 complex. In the ribbon diagram of ternary complex, D1 domain of suPAR is shown in orange, D2 domain in magenta, D3 domain in green, uPA¹⁻¹⁴³ in cyan, light chain of ATN-615 in light blue and heavy chain in blue. Carbohydrates in suPAR are shown in red sticks. Shown in dashed lines are disulfide bonds in suPAR (12 bonds), uPA¹⁻¹⁴³ (5 bonds), and Fab ATN-615 (4 bonds).

FIG. 1B shows a 2Fo-Fc Electron density map of the residues (T18-W30) of uPA¹⁻¹⁴³ contoured at 1 σ.

FIG. 1C: uPA¹⁻¹⁴³ structure (in cyan) of the present invention was superimposed with structures of soluble uPA¹⁻¹⁴³ (other colors) from NMR analysis using the Cα atoms from the KrD. The GFD (uPA¹¹⁻⁴⁷) and the KrD (uPA⁴⁸⁻¹³⁰) are associated through residues L14, H41, I44, D45, R59, L92 and Y101—shown as sticks. The Ω-loop (uPA²³⁻²⁹) links two β-strands (uPA¹⁸⁻²² and uPA³⁰⁻³²) in the GFD. Two short α-helices (uPA⁷⁸⁻⁸¹ and uPA⁹¹⁻⁹⁴) and two β-strands (uPA¹¹²⁻¹¹⁷ and uPA¹²⁰⁻¹²⁵) with extended loops are found in the KrD.

FIG. 2A shows the domain structure of suPAR in the suPAR-uPA¹⁻¹⁴³ complex. Domain D1, uPAR¹⁻⁸⁰, includes six β-strands (β1: residues 2-7, β2: 10-16, β3: 23-32, β4: 38-46, β5: 53-58, β6: 63-71). Domain D2, uPAR⁹³⁻¹⁹¹ (magenta) includes six β-strands (β7: 94-99, β8: 111-114, β9: 121-128, β10: 143-149, β11: 156-161, β12: 164-171) and a short α-helix (α1: 104-107). Domain D3, uPAR¹⁹²⁻²⁸³ (green) includes five β-strands (β13: 193-198, β: 211-214, β: 220-226, β: 237-242, β17: 262-266) and two short α-helices (α2: 244-246 and α3: 253-256).

FIG. 2B shows the association of D1 and D2 domains. The β5 strand in D1 is essential for D1-D2 association. Residues involved in the D1-D2 interaction are shown in sticks.

FIG. 2C shows superimposition of the D1-D2 domains of uPAR as observed in the present invention (orange and magenta) on a suPAR structure to which is bound an antagonist peptide (Llinas, P, M H Le Du, et al (2005) Embo J 24:1655-63), shown in gray. The two structures were aligned using the Cα atoms from the D2D3 domains. Domain D1 shows 20.5° rotation and 10 Å movement between the two structures.

FIG. 2D shows D1-D3 domain interface variation between uPA¹⁻¹⁴³-bound uPAR. D1 and D3 are depicted in orange and green, respectively and peptidyl inhibitor-bound suPAR (gray) (Llinas et al., supra). Residues H47, E49, K50, R53, L252, D254, N259 and H260 involved in D1-D3 interactions are shown in sticks. Peptide binding causes movement of the uPAR D3 domain by up to 9.5 Å, and disrupts the D1-D3 association.

FIG. 3A-3C depicts the uPAR-uPA¹⁻¹⁴³ binding surface. Carbon atoms of uPAR D1 are depicted in orange, D2 in magenta, and D3 in green. uPA¹⁻¹⁴³ is depicted as a ribbon diagram in cyan. FIG. 3A shows molecular surface representation of overall uPAR-uPA¹⁻¹⁴³ binding. The three uPAR domains form a cone shaped cavity with a wide opening (25 Å) and a depth of 14 Å and are involved in the uPA¹⁻¹⁴³ binding. FIG. 3B shows a detailed surface representation of uPAR-uPA¹⁻¹⁴³ binding. Elements: O is red, N is blue and S is yellow. H₂O is depicted as red spheres. Light blue dashed lines indicate hydrogen bonds. FIG. 3C shows detailed interaction of suPAR (ribbon) and uPA¹⁻¹⁴³. T8, R53, E68, T127 and 166H of uPAR form hydrogen bonds with S21, K23, Y24, S26 and Q40 of uPA¹⁻¹⁴³.

FIG. 4 is a table showing x-ray crystallographic details and coordinates.

### DESCRIPTION OF THE PREFERRED EMBODIMENTS

The present inventors conceived of adding a uPAR binding partner, such as a uPAR-specific antibody or fragment (e.g., an Fab fragment) to constrain the 3D structure and thereby facilitate crystallization and improve the diffraction resolution of uPAR-uPA¹⁻¹⁴³ complexes. The mAb ATN-615, which had been raised by some of the present inventors against suPAR and which binds to suPAR at a domain so that such binding does not disrupt uPA-uPAR interactions was used in this capacity and is exemplified herein. Indeed, the ATN-615 Fab fragment facilitated suPAR-uPA¹⁻¹⁴³ crystallization, greatly improved the diffraction resolution of the crystals to 1.9 Å and provided phasing power to generate a discernible electron density map for suPAR and uPA¹⁻¹⁴³ model building.

Antibodies are not the only type of uPAR binding partners that may be used in the present invention. Any ligand for uPAR that binds to uPAR without disrupting the binding of uPA to uPAR or altering the structure of the uPA-uPAR complex may be used for similar structural analysis. Examples of suPAR binding partners are vitronectin (Vn) and various integrins. Similarly, any binding partner that binds to uPA without altering its structure or interfering with its binding to uPAR may also be used for structural analysis. Other ligands useful as above include peptides, phages, small organic molecules, aptamers, and the like that bind either to suPAR or uPA.

By enabling a structural determination of the uPA-uPAR binding interaction at a new level of resolution, the present invention enables the testing and screening of potential inhibitors or antagonists of this interaction. First, the restrictive species specificity of uPA-uPAR interactions is an impediment to testing inhibitors in murine or other rodent systems, for example. The present structure shows that residues involved in the first region of the uPAR-uPA interface are highly conserved among different species so that antagonists targeting this region would inhibit both human and mouse uPAR-uPA interactions.

The structure of uPAR-uPA¹⁻¹⁴³ complex described herein serves as a platform for rational design of inhibitors of uPAR-uPA interactions.

Having now generally described the invention, the same will be more readily understood through reference to the following examples which are provided by way of illustration, and are not intended to be limiting of the present invention, unless specified.

### Example I

### Materials and Methods

### Recombinant Expression of Polypeptides and Formation of Complexes.

uPAR and uPA¹⁻¹⁴³ (amino acid residue 1-143 uPA) were expressed in drosophila S2 cells and purified as described (Huang, M, A P Mazar, et al (2005) Acta Crystallogr D Biol Crystallogr 61(Pt 6):697-700). The suPAR-uPA¹⁻¹⁴³ complex was formed by incubating uPA¹⁻¹⁴³ with suPAR at a 1:1 molar ratio at room temperature in 50 mM HEPES and 100 mM NaCl pH 7.4 and was purified on a Superdex75 gel filtration column. The suPAR-uPA¹⁻¹⁴³ complex was then mixed at 1:1 molar ratio with Fab fragment of anti-suPAR antibody, ATN-615, and the mixture was purified on a superdex 200 column. The 1:1:1 suPAR-uPA¹⁻¹⁴³ Fab was concentrated to 10 mg/ml using Millipore Ultrafree centrifugal filters.

### Generation of Crystals

Diffracting quality crystals of the suPAR-uPA¹⁻¹⁴³-ATN-615 ternary complex were generated by microdialysis (McPherson, A., Preparation and Analysis of Protein Crystals, John Wiley & Sons, 1992, pp 88-91) with 4% PEG4K, 5% ethylene glycol, 5% methanol, 0.05% sodium azide, 50 mM cacodylate pH 6.5. The crystals typically appeared in 3 to 7 days, and grew to a maximal size of 0.03×0.05×0.1 mm³. The crystals are harvested from dialysis button, and brief soaked in a cryoprotectant of 20% glycerol, 20% PEG4K, 5% methanol, 50 mM cacodylate pH 6.5.

### X-Ray Crystallography, Analysis and Model Building

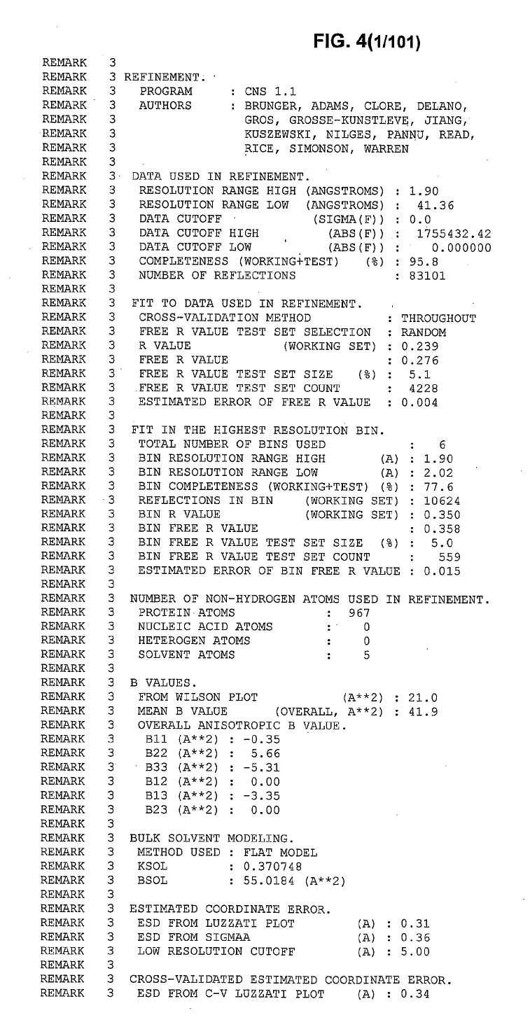

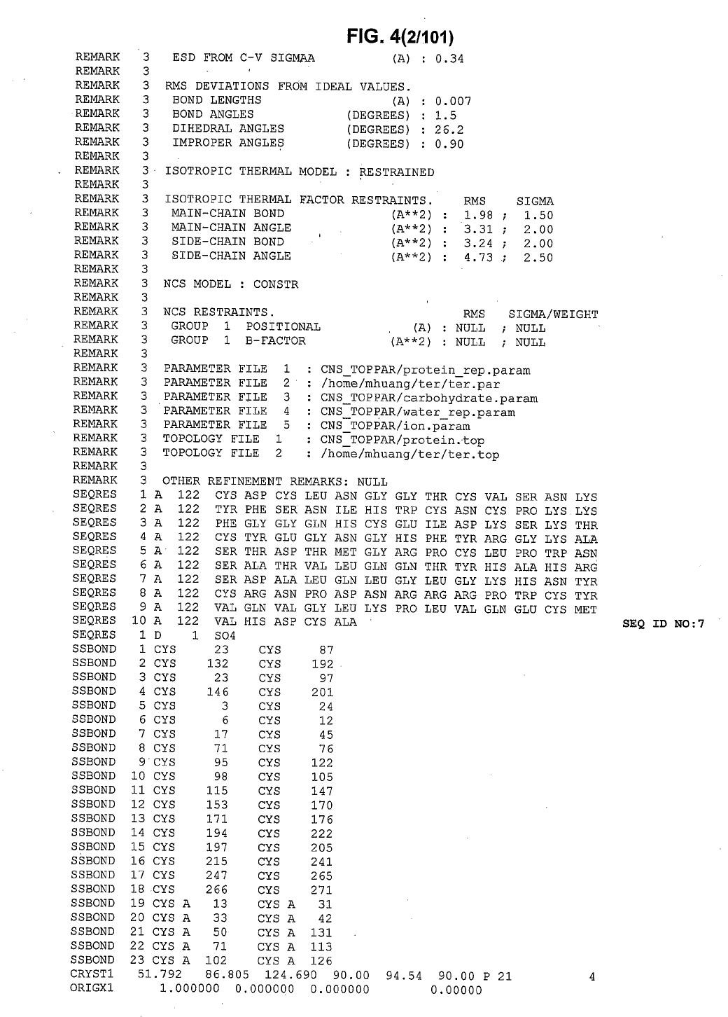

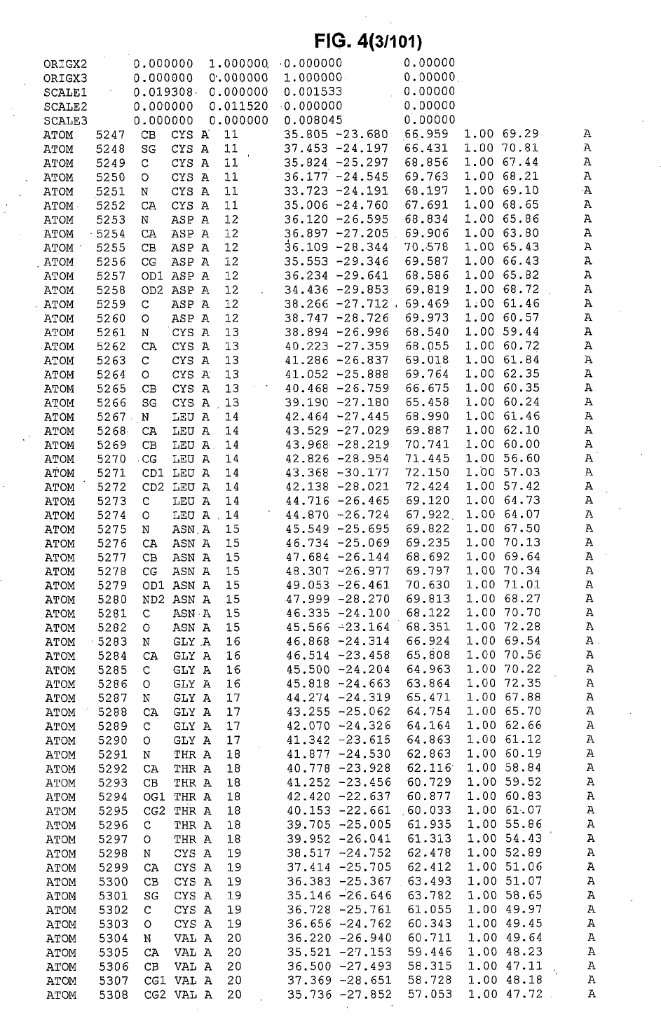

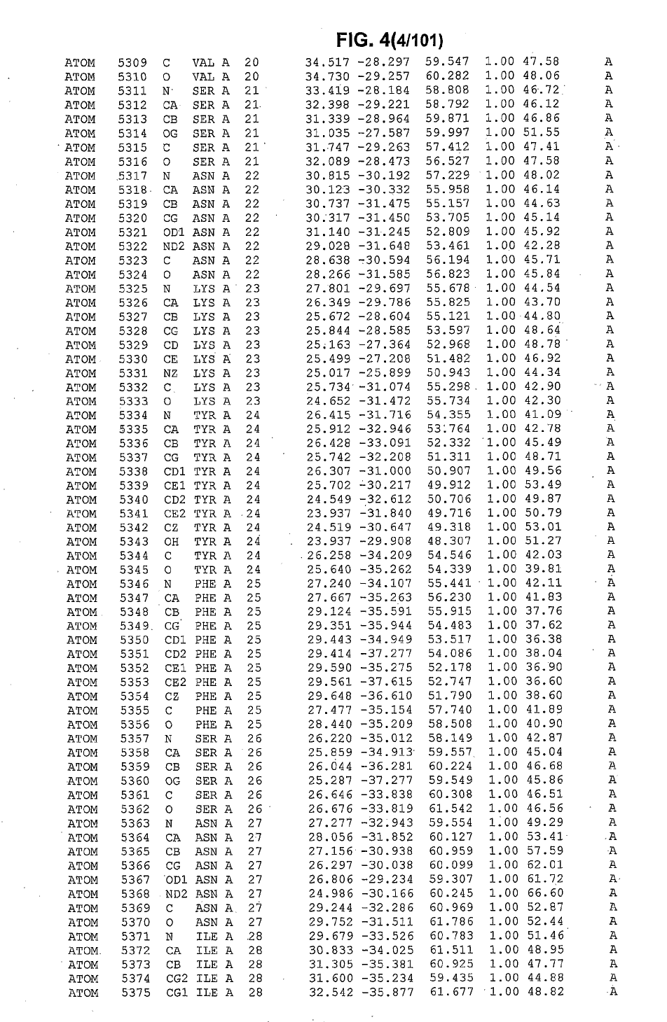

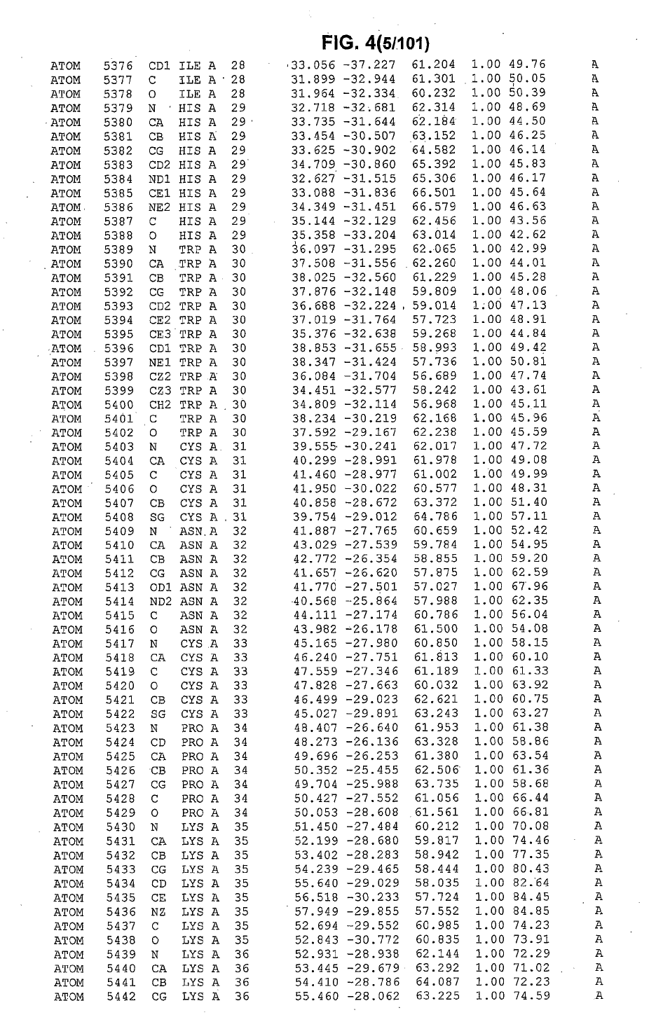

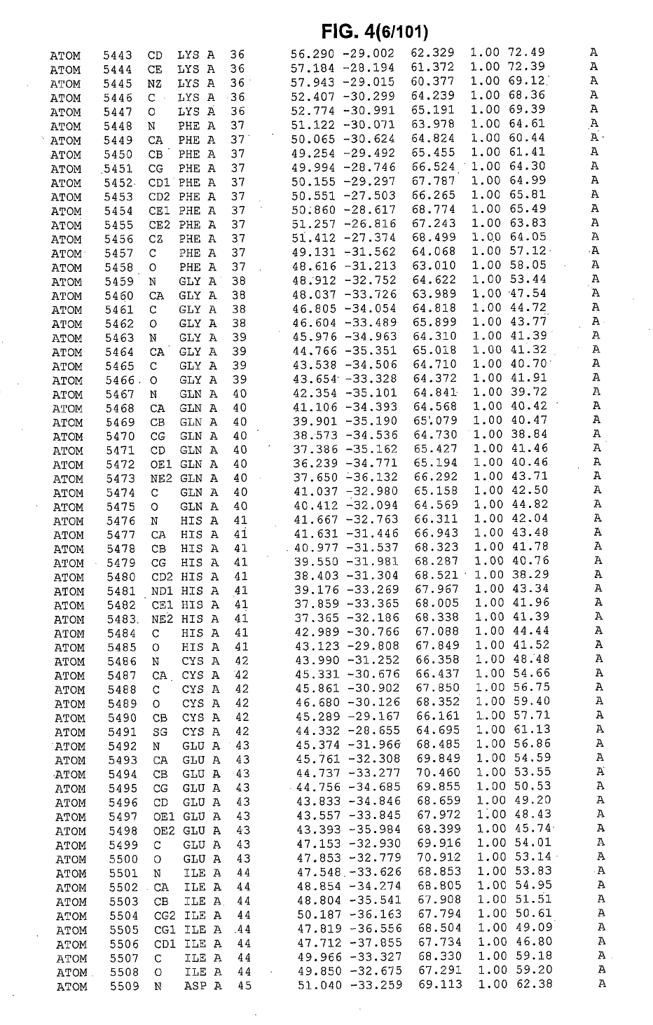

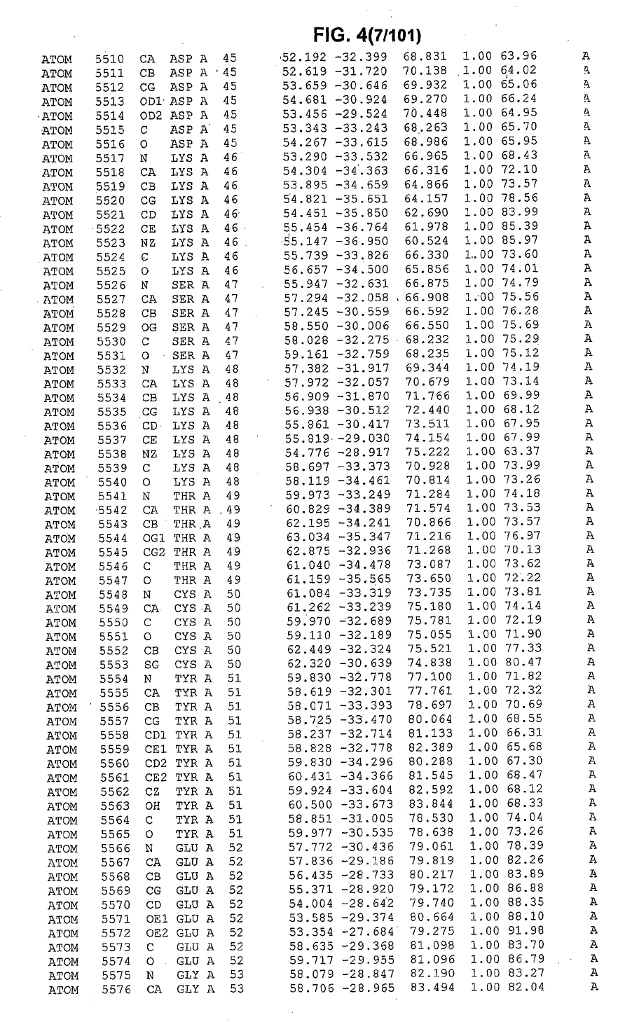

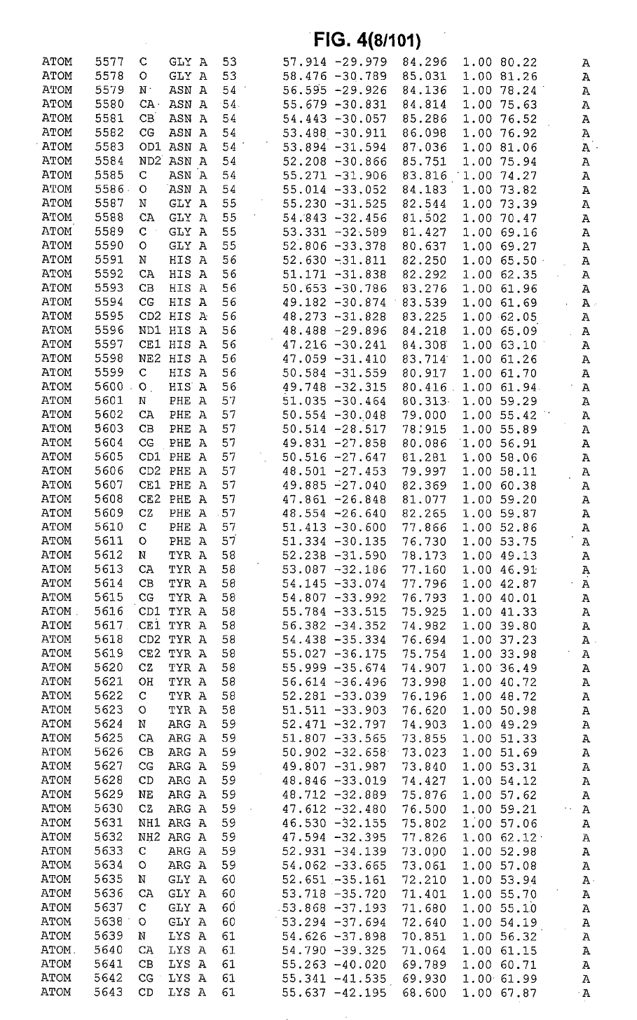

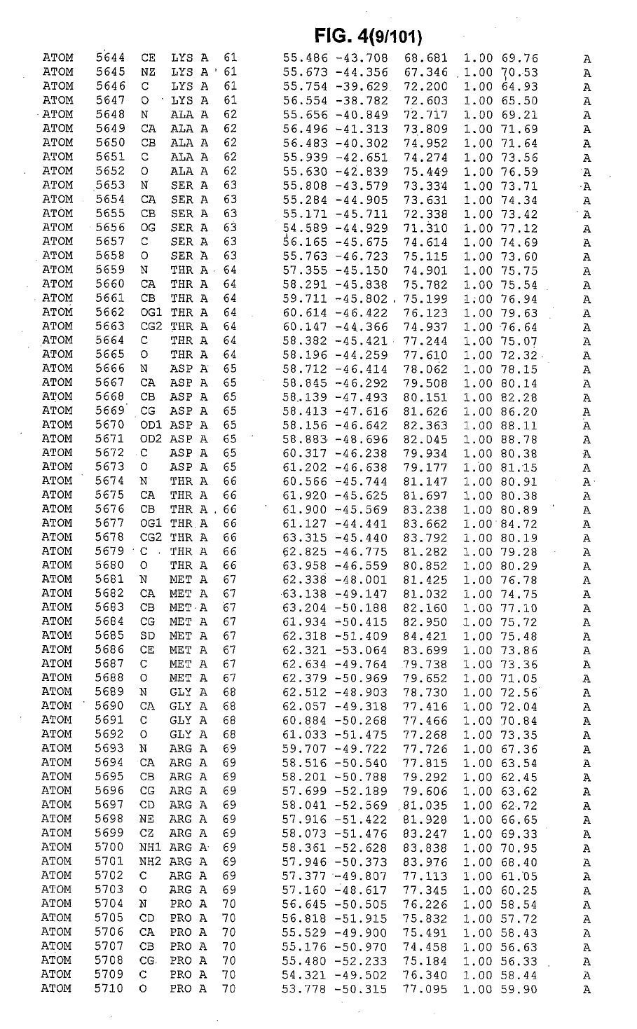

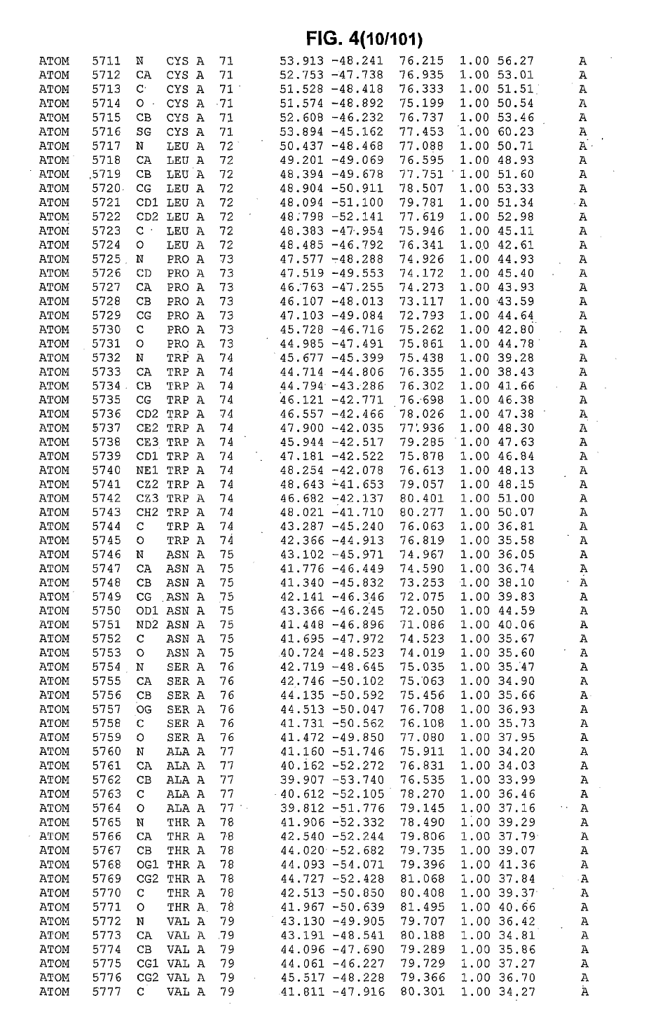

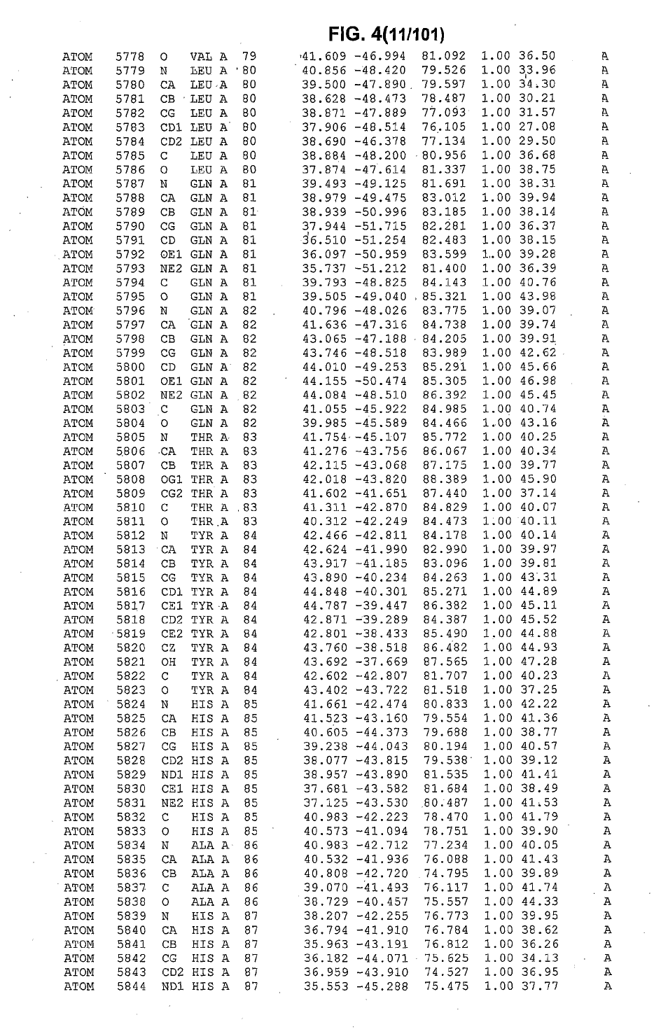

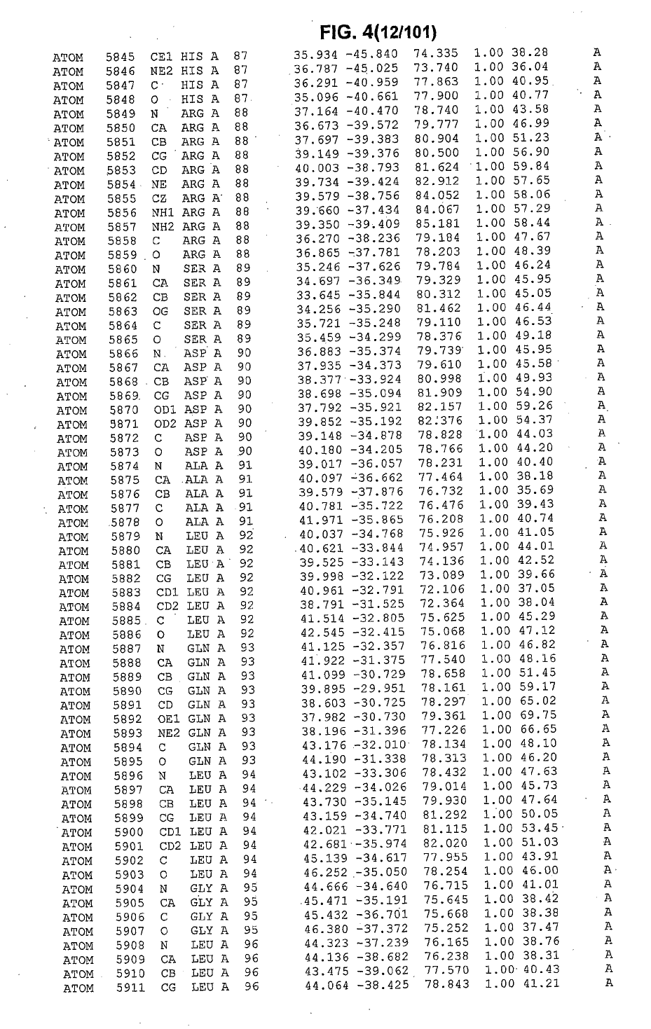

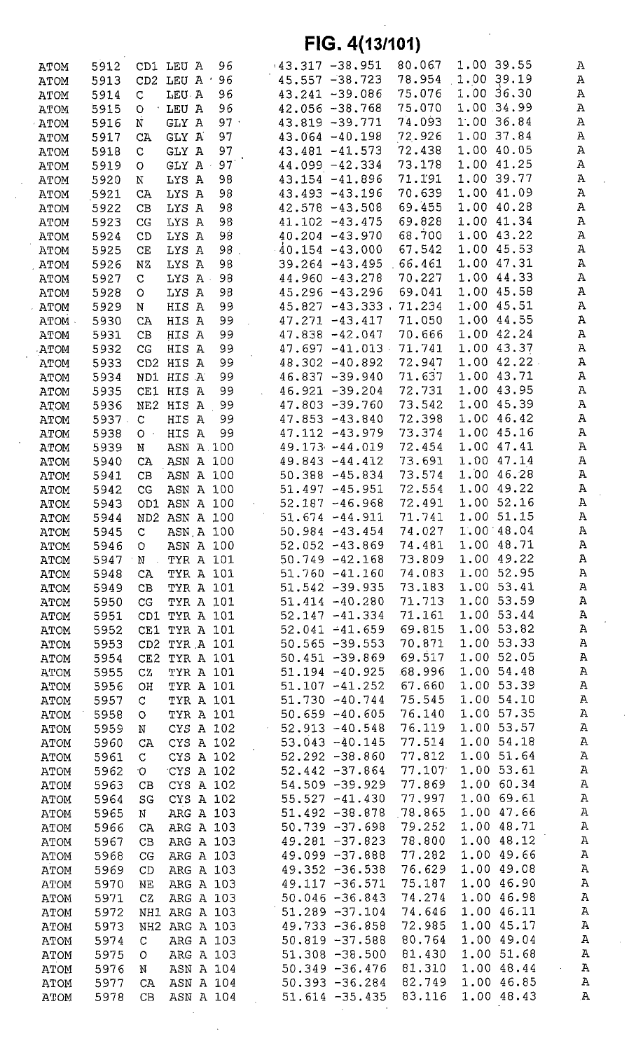

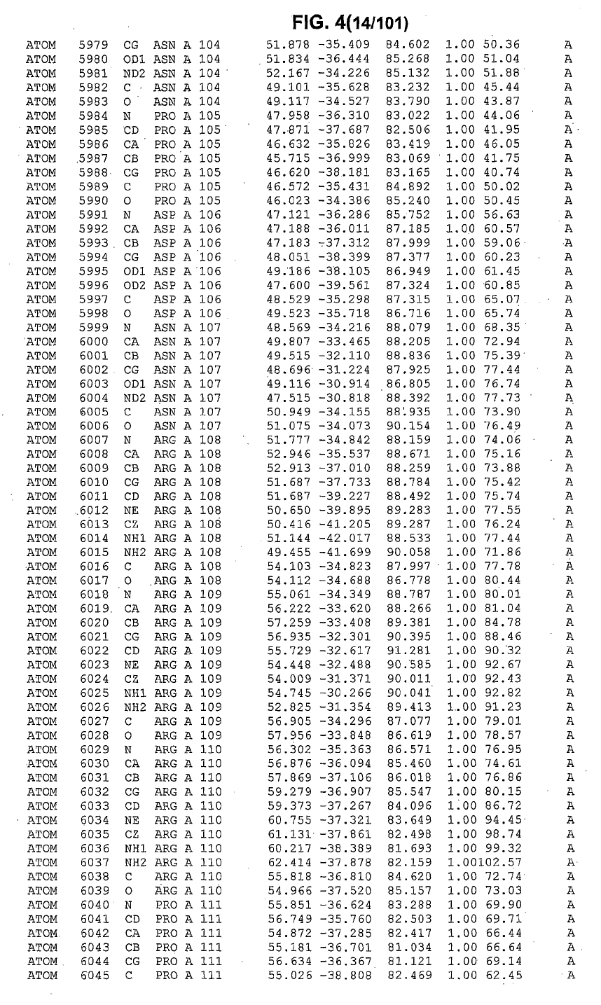

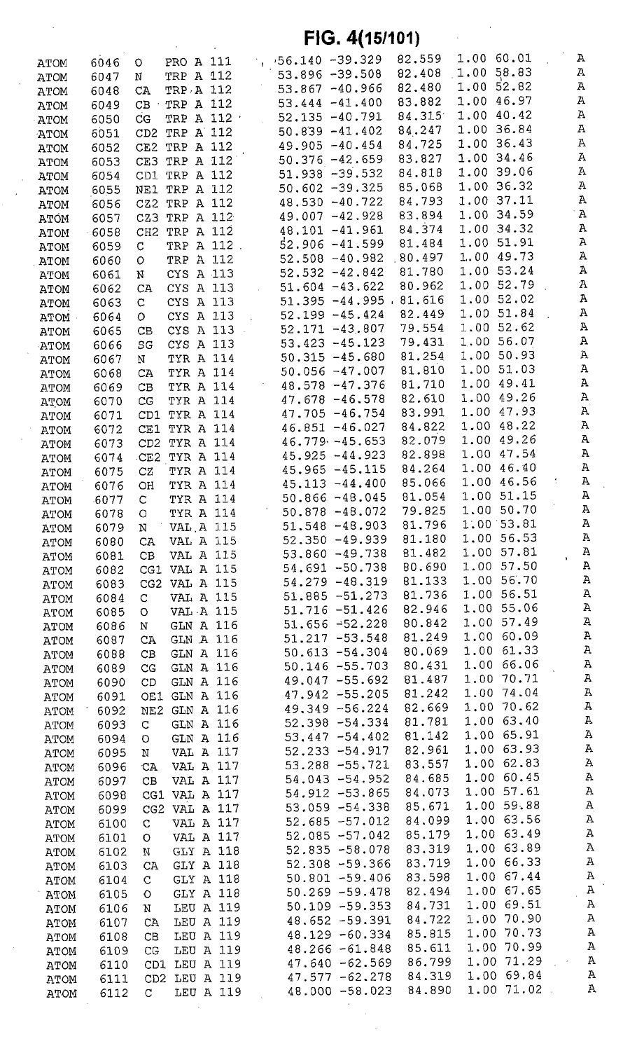

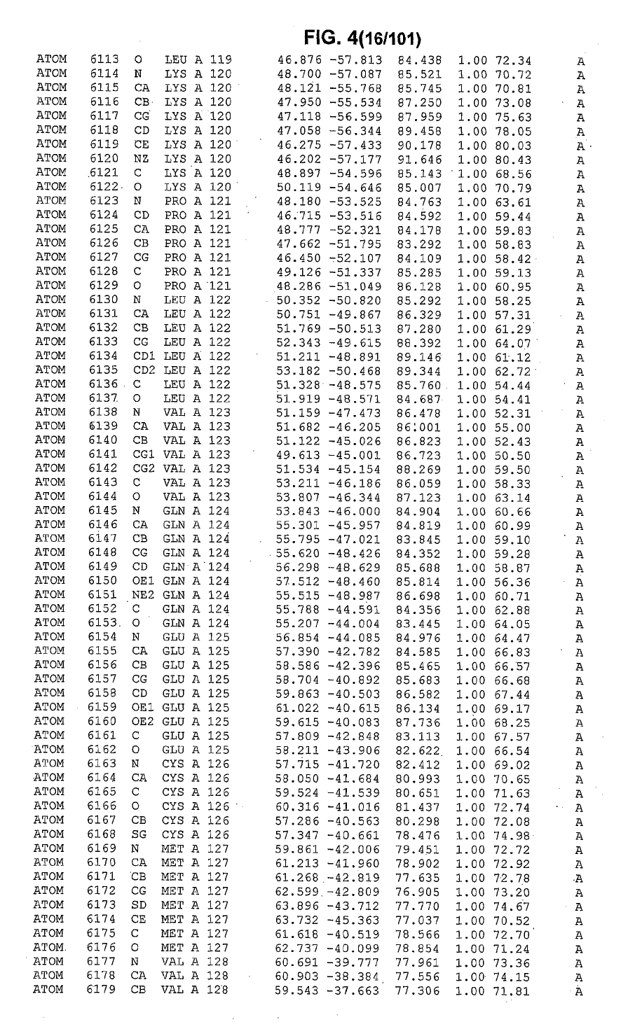

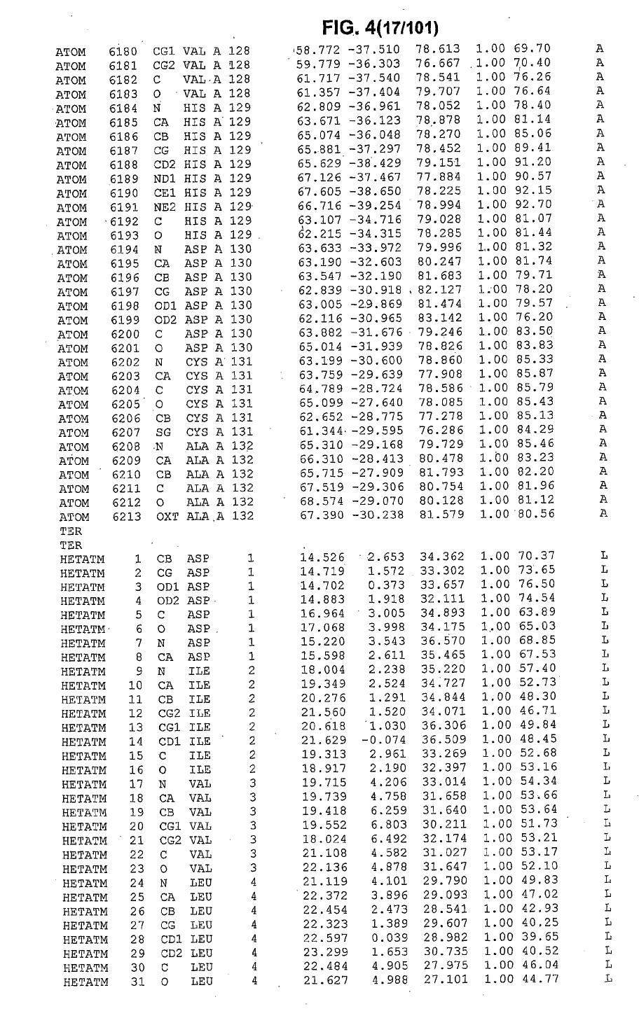

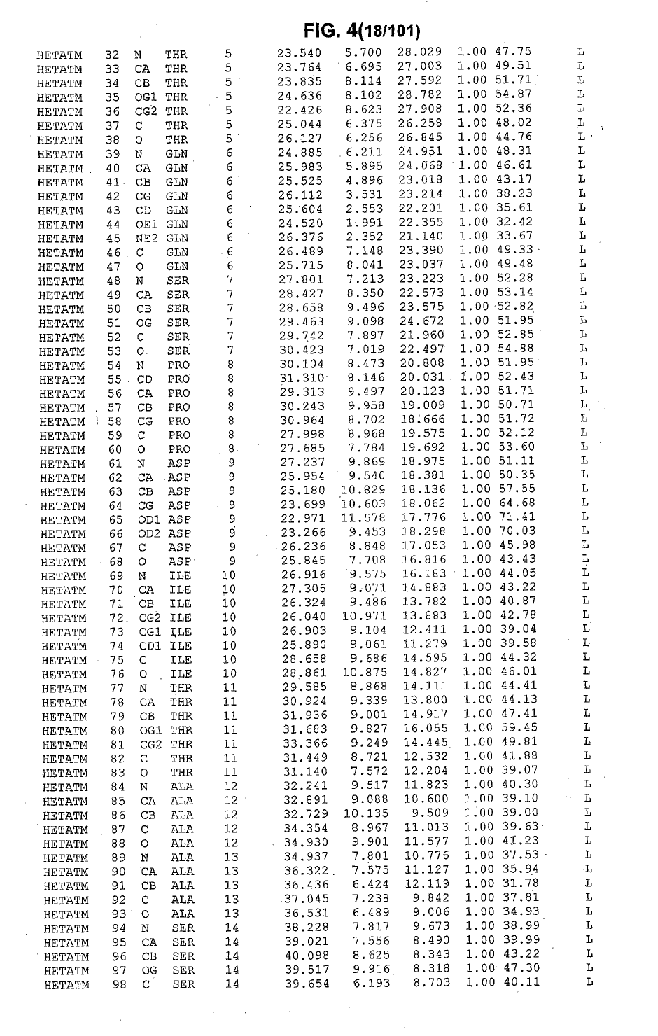

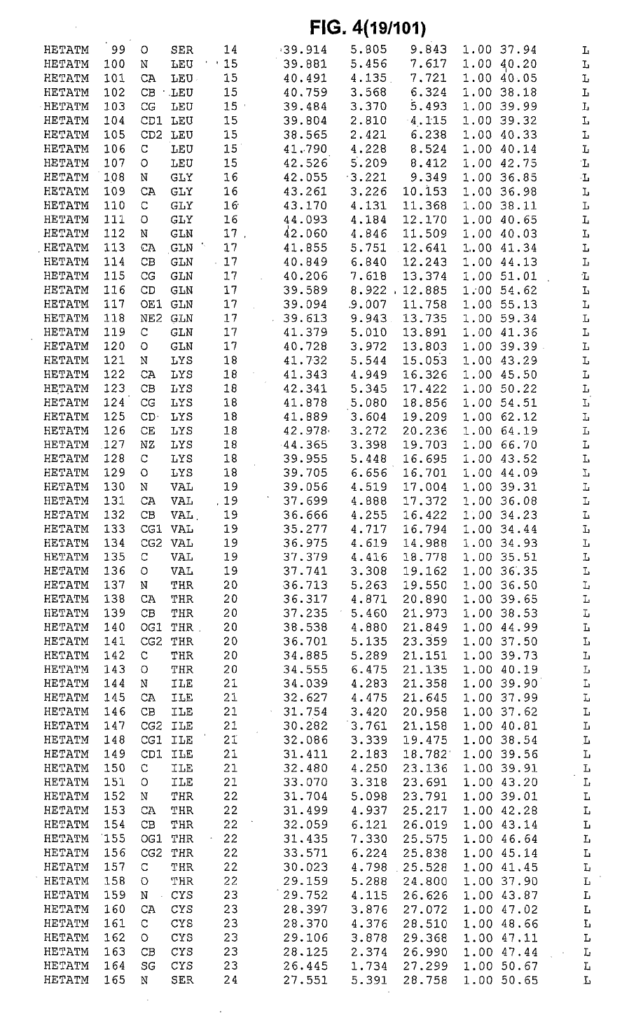

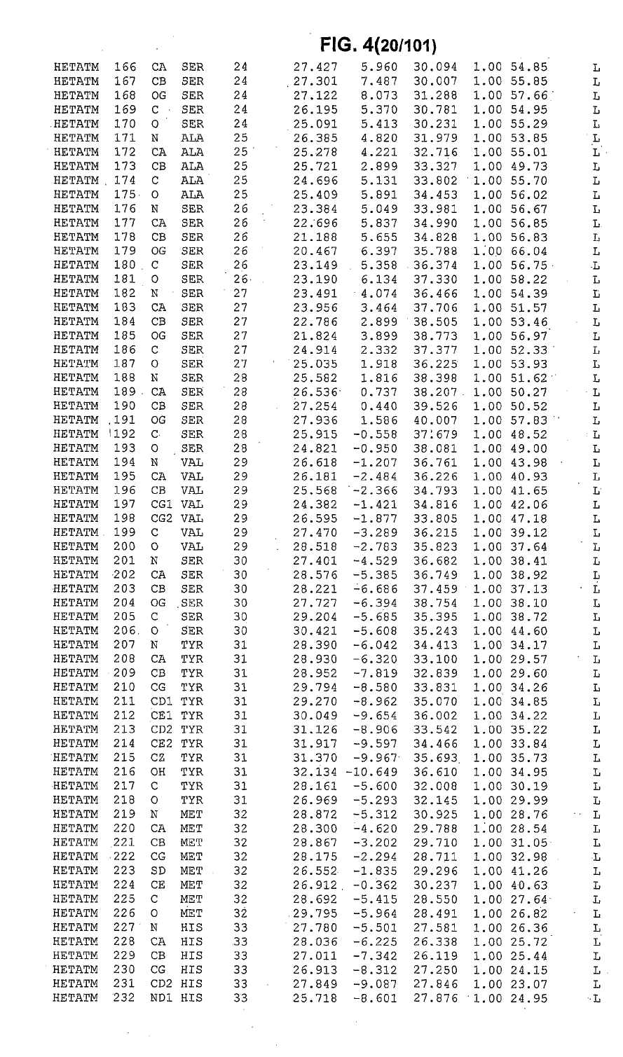

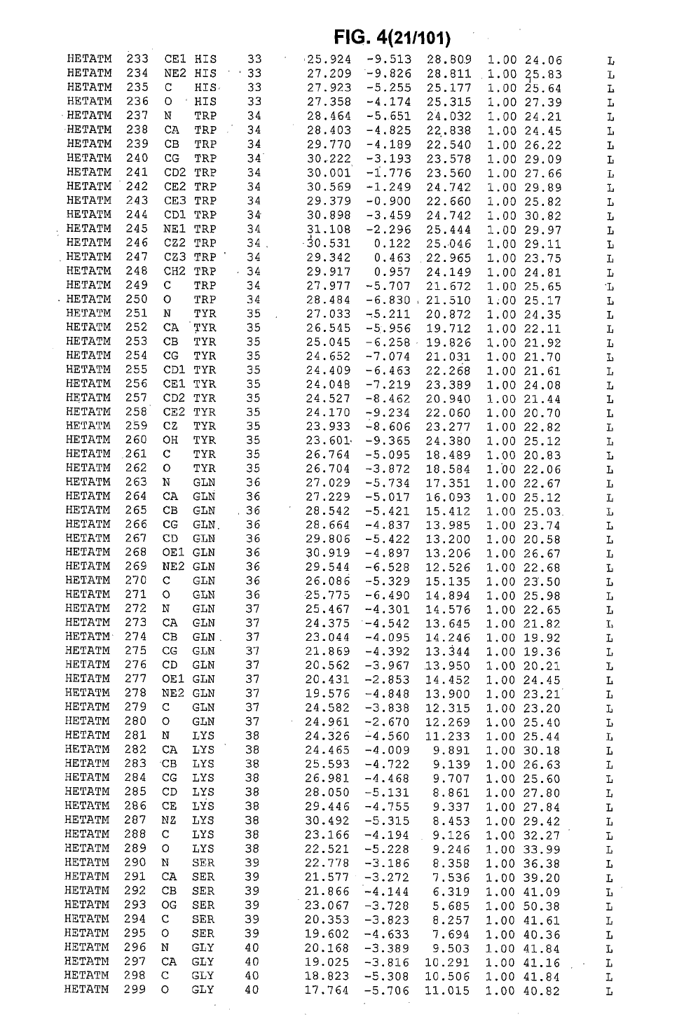

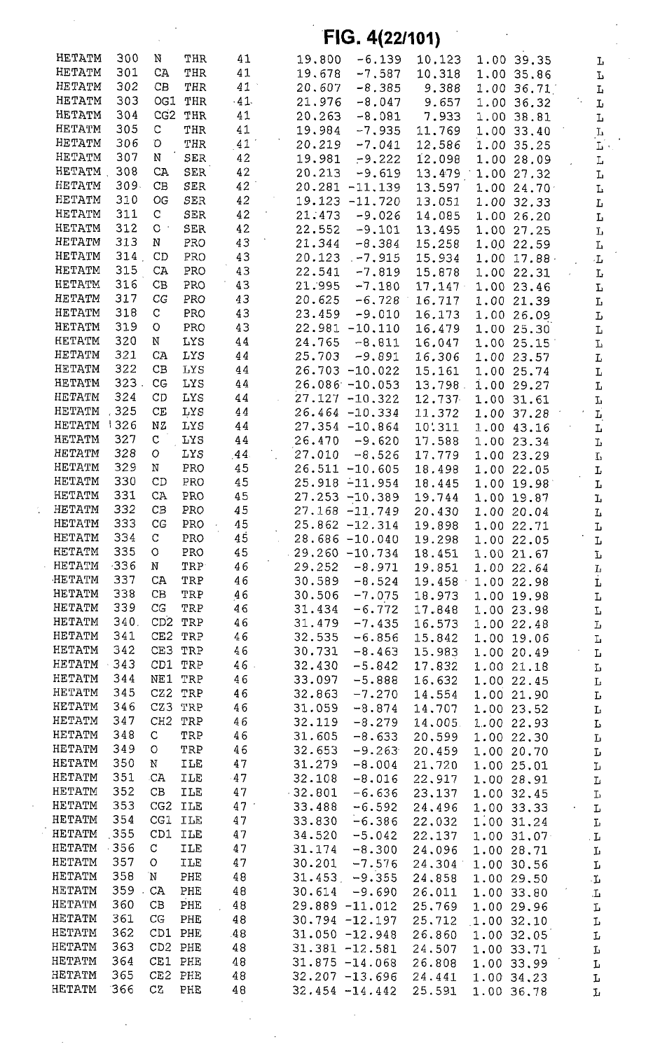

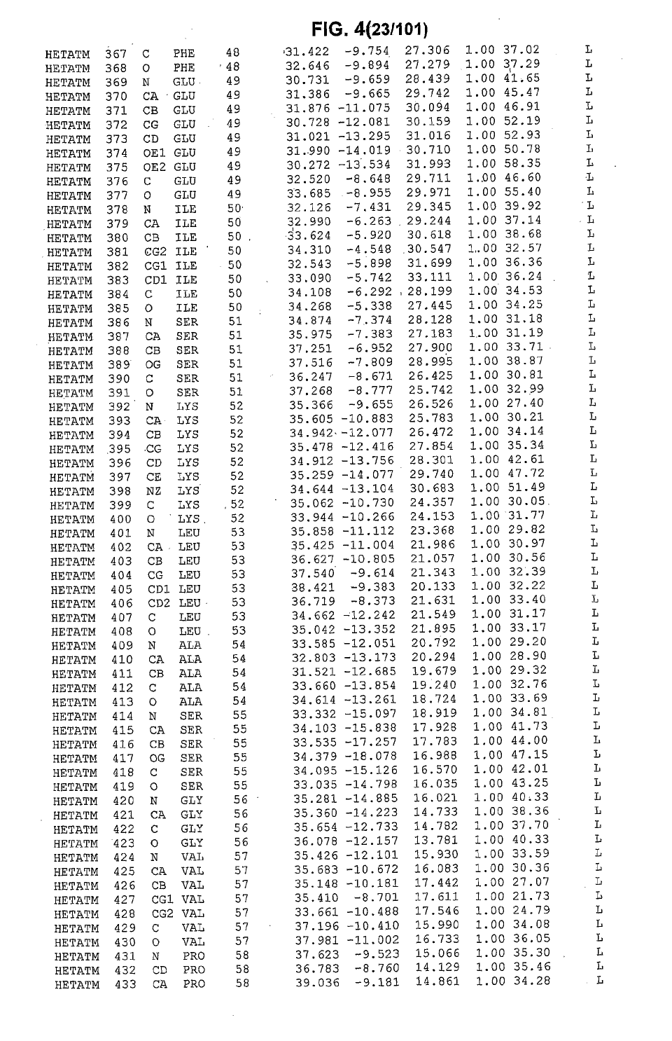

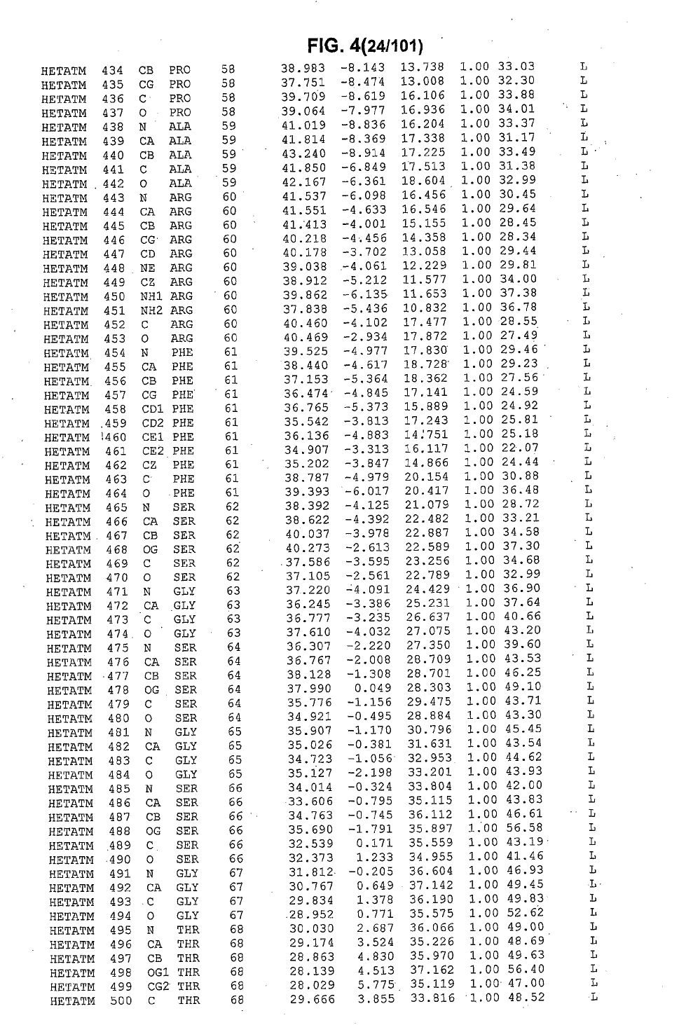

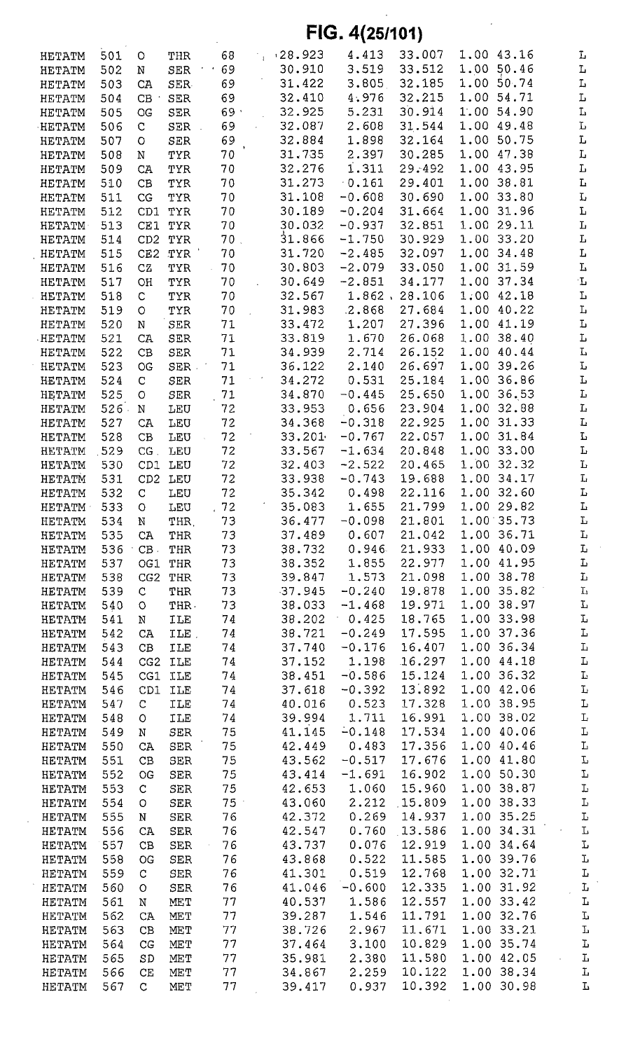

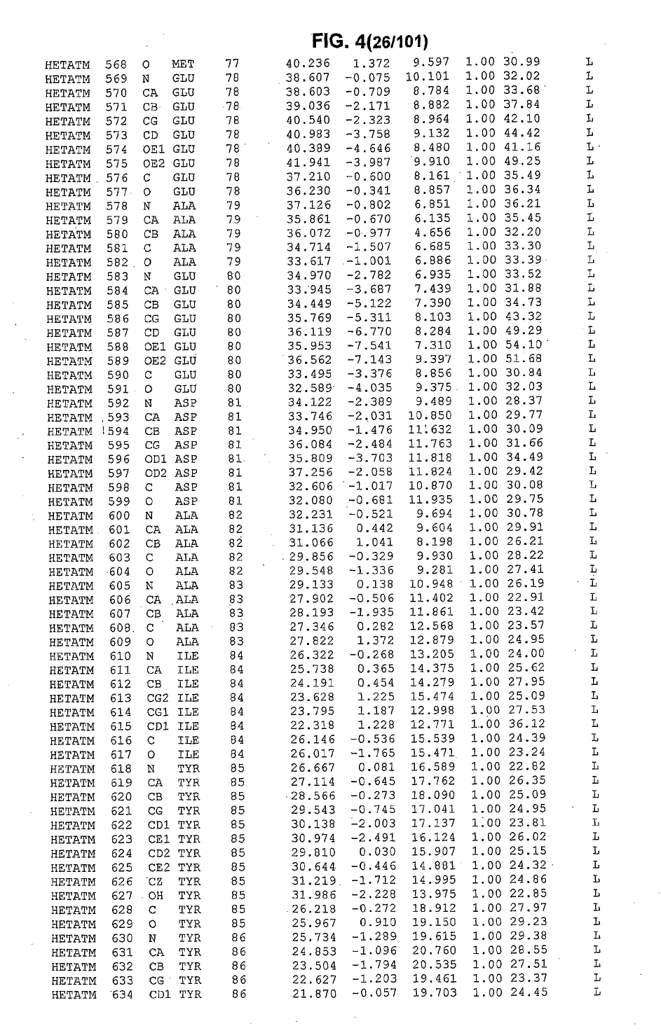

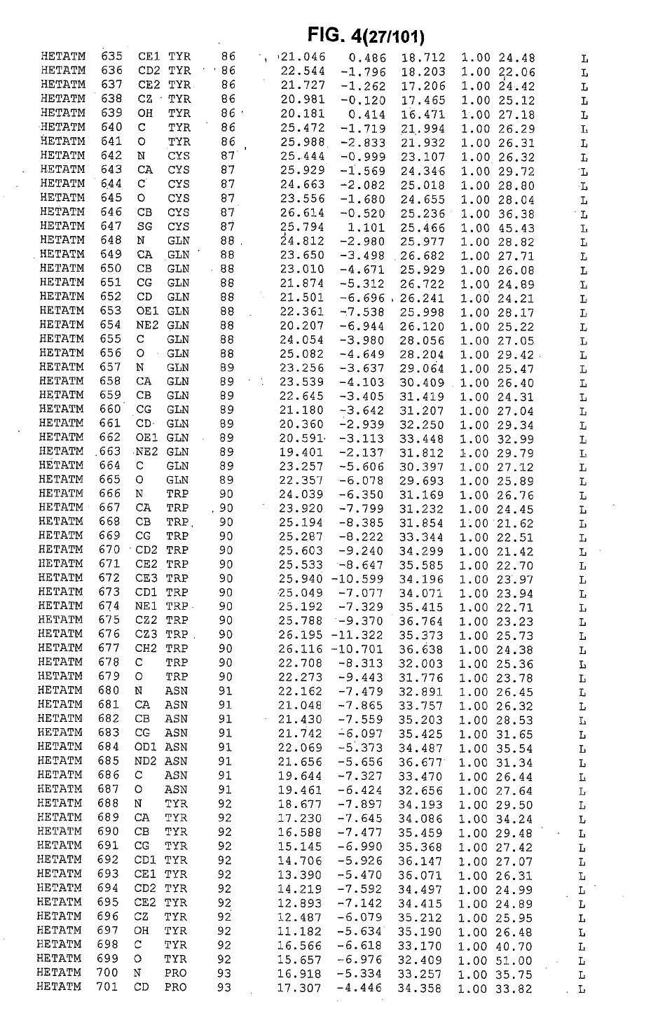

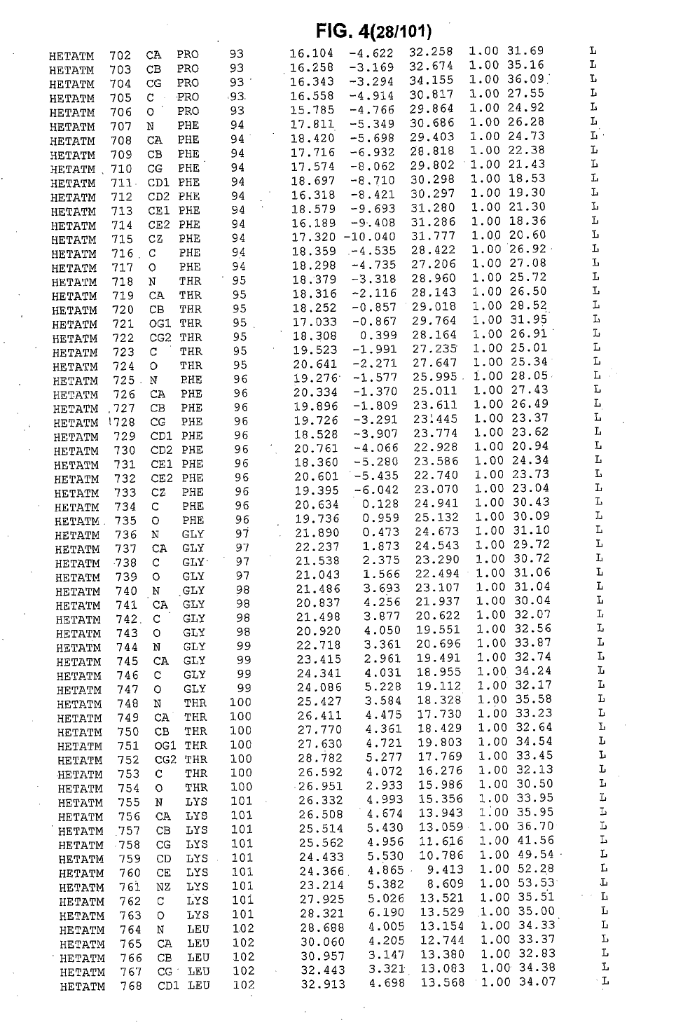

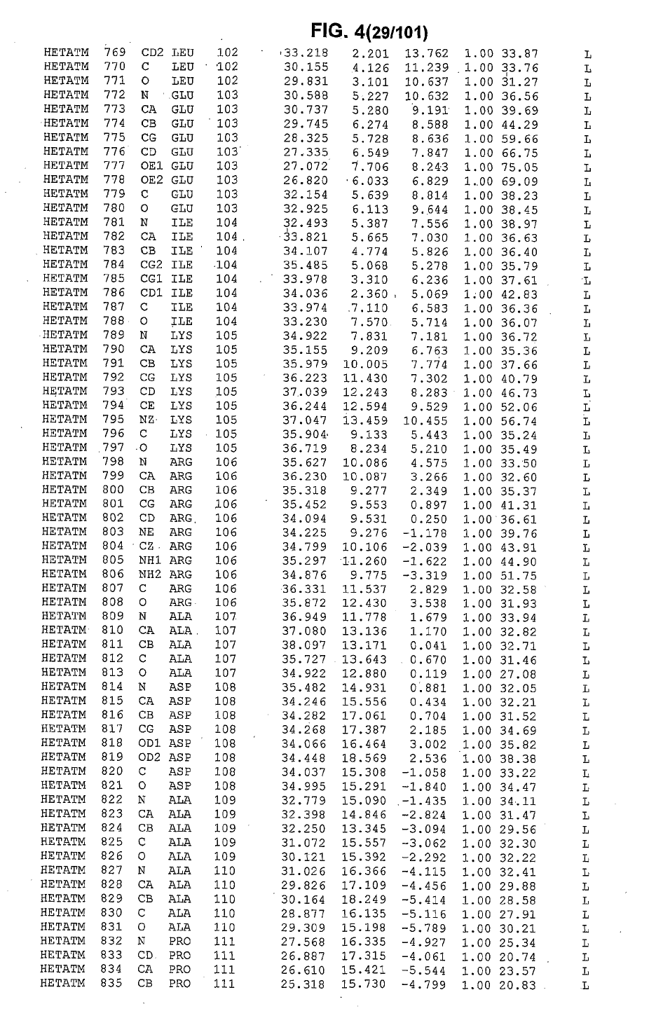

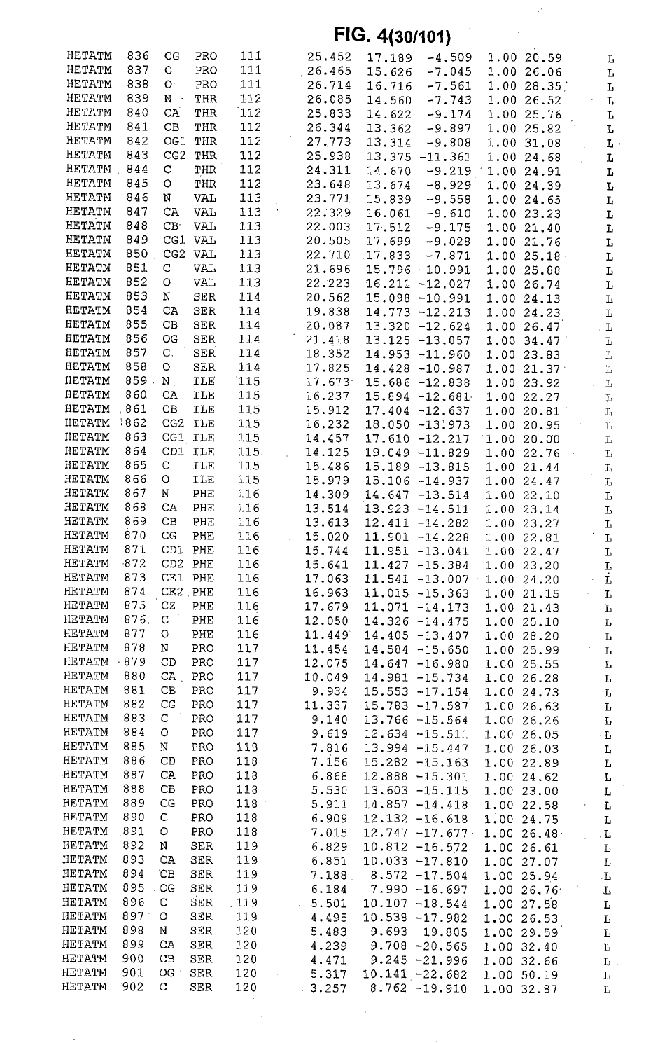

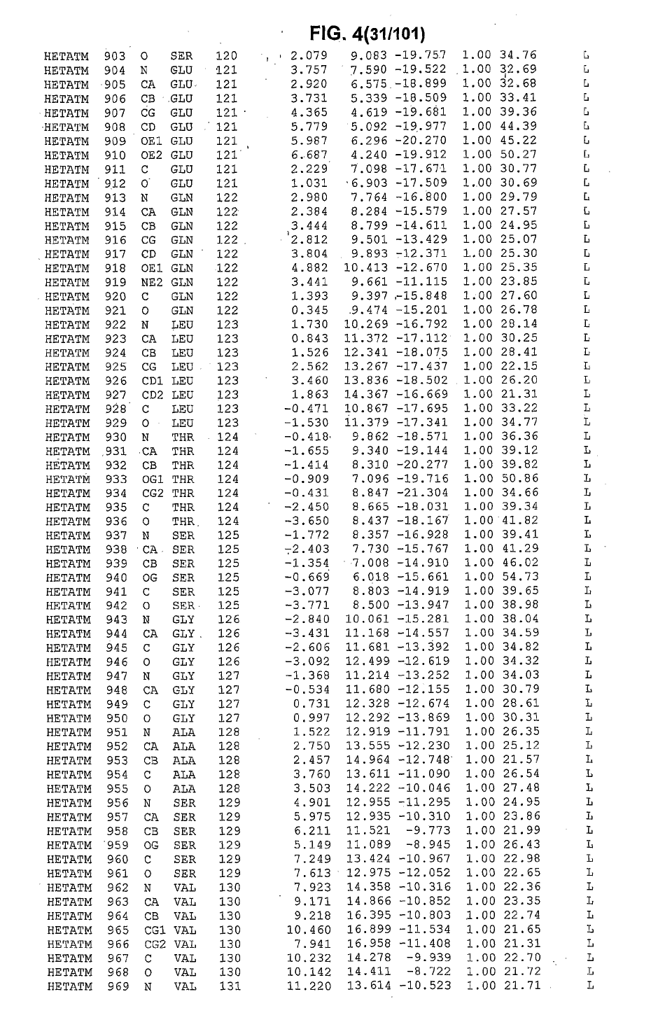

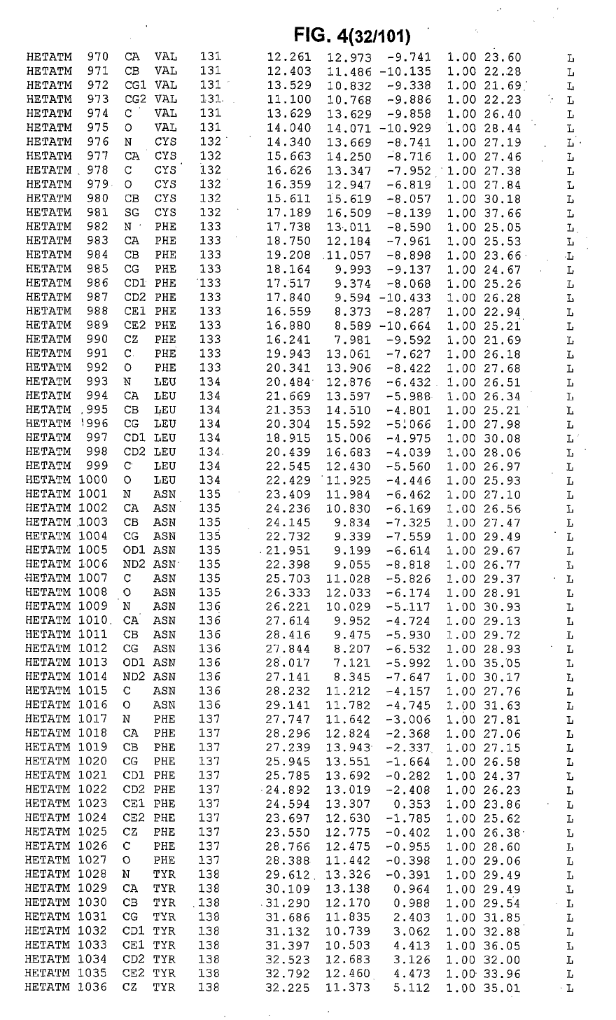

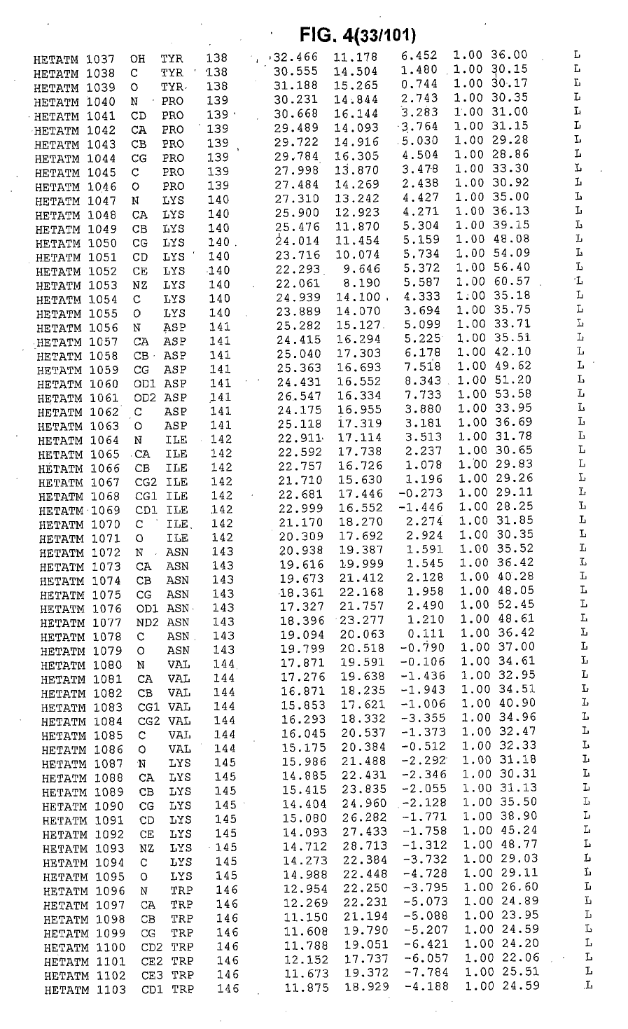

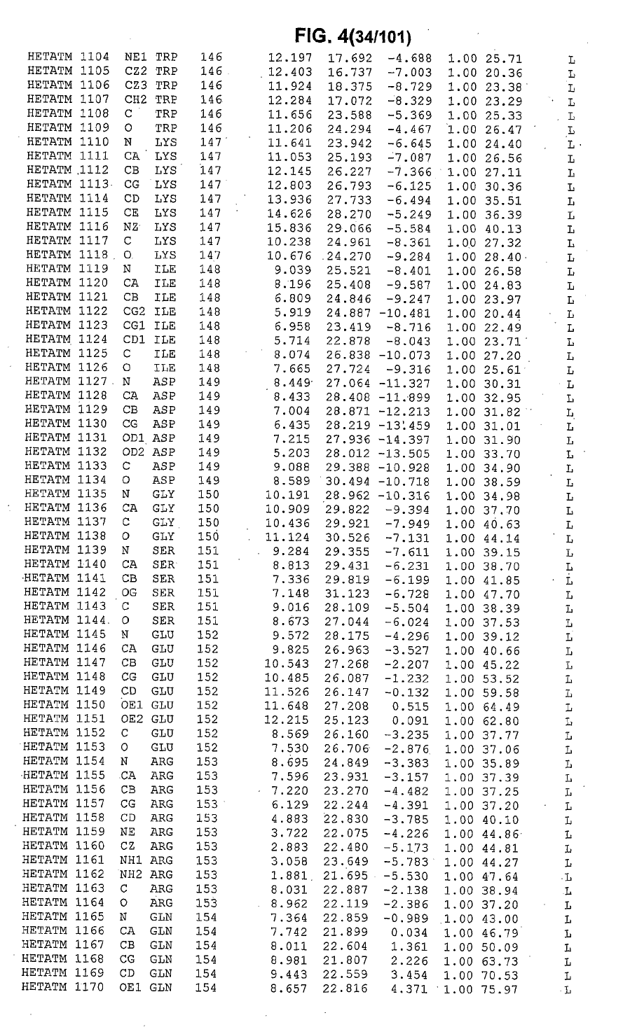

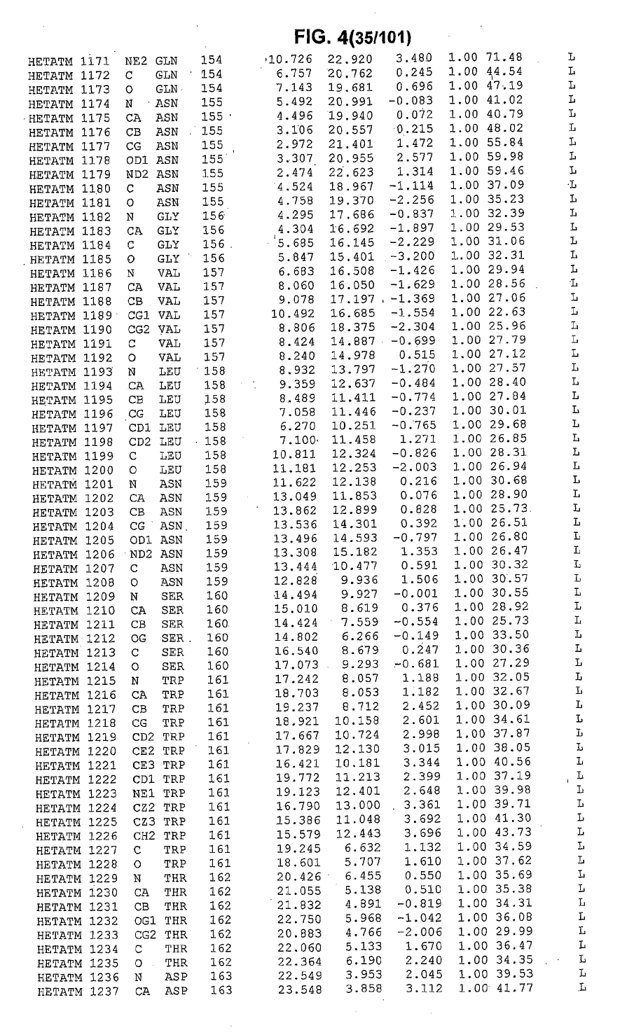

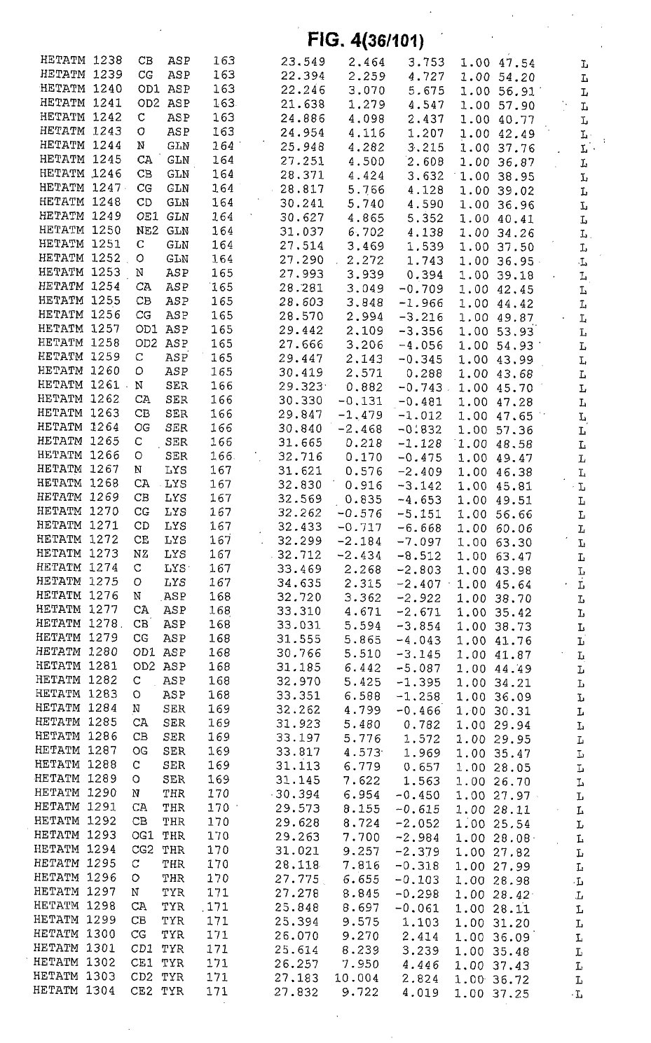

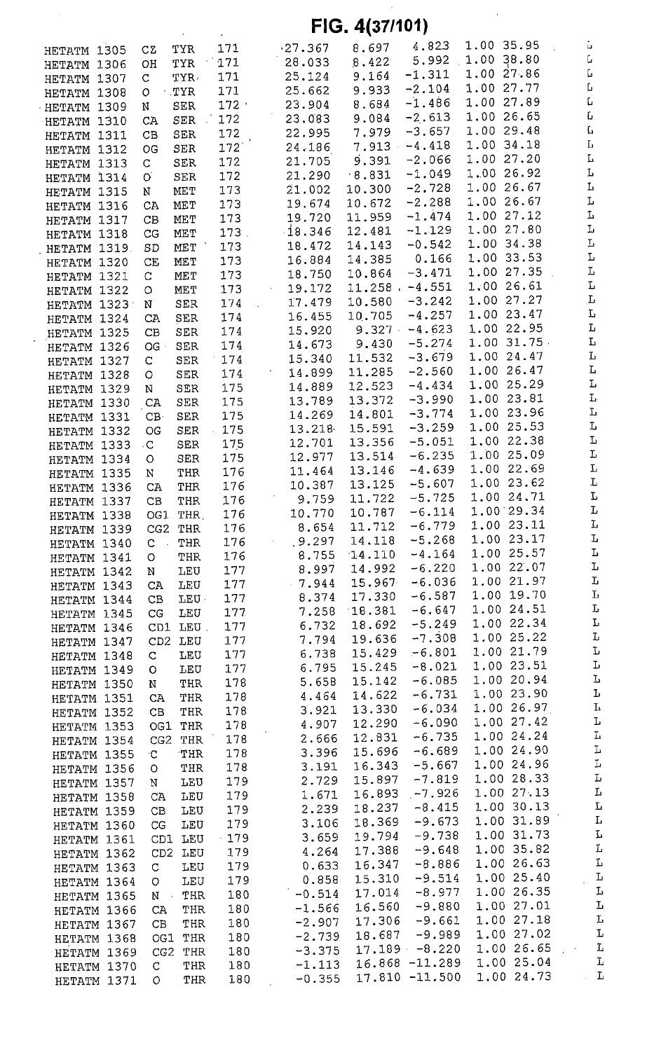

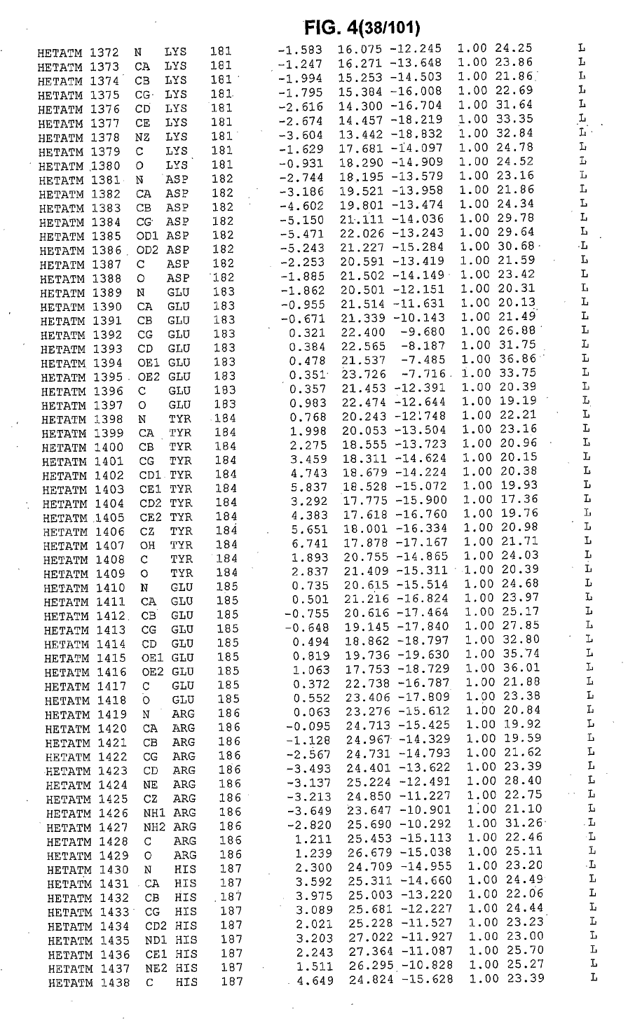

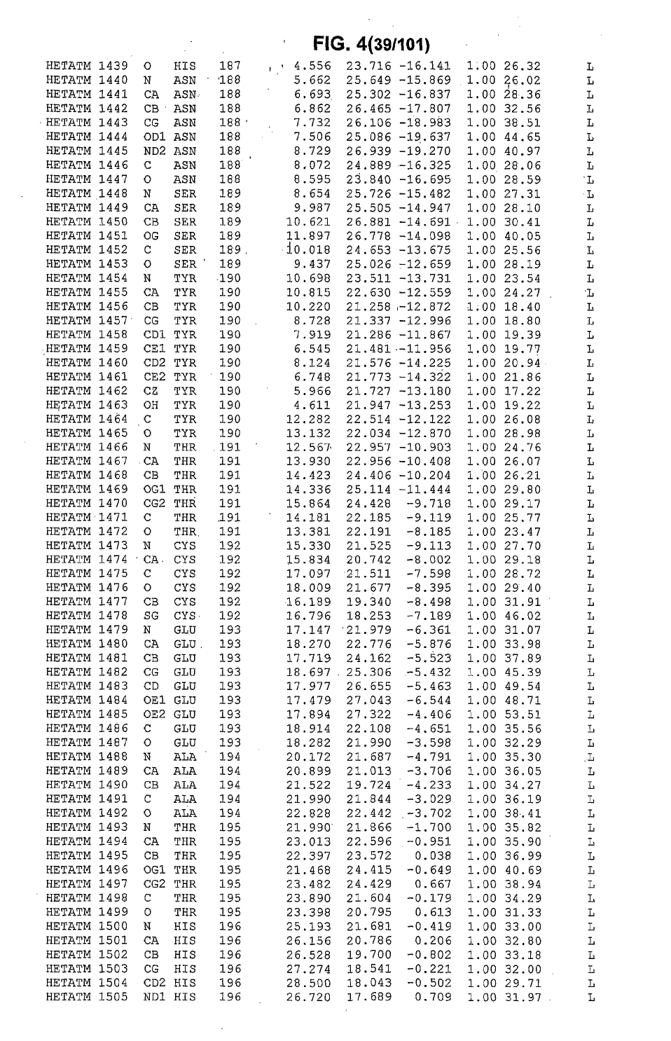

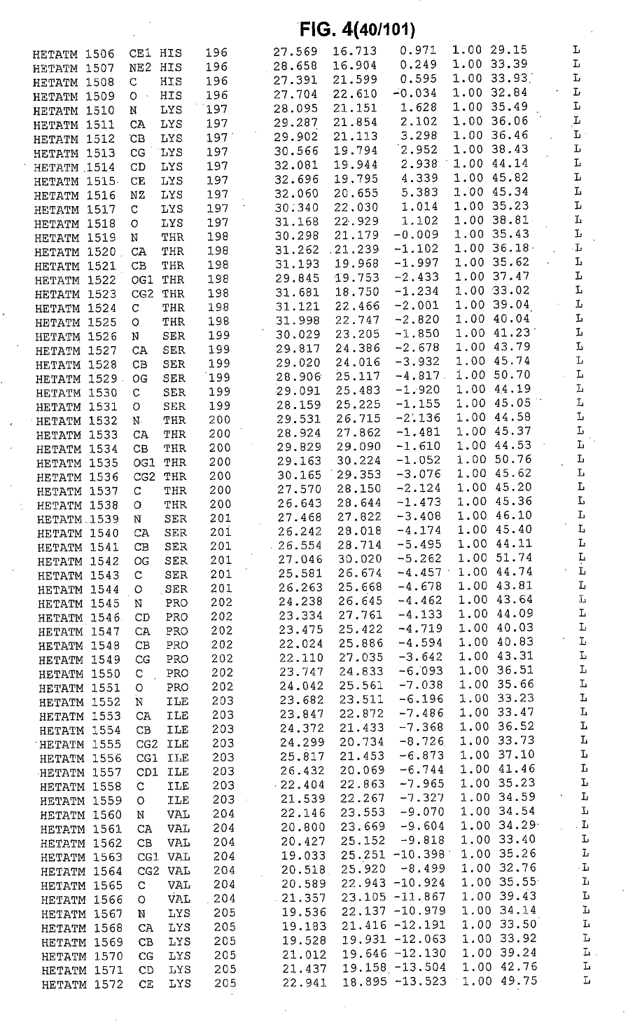

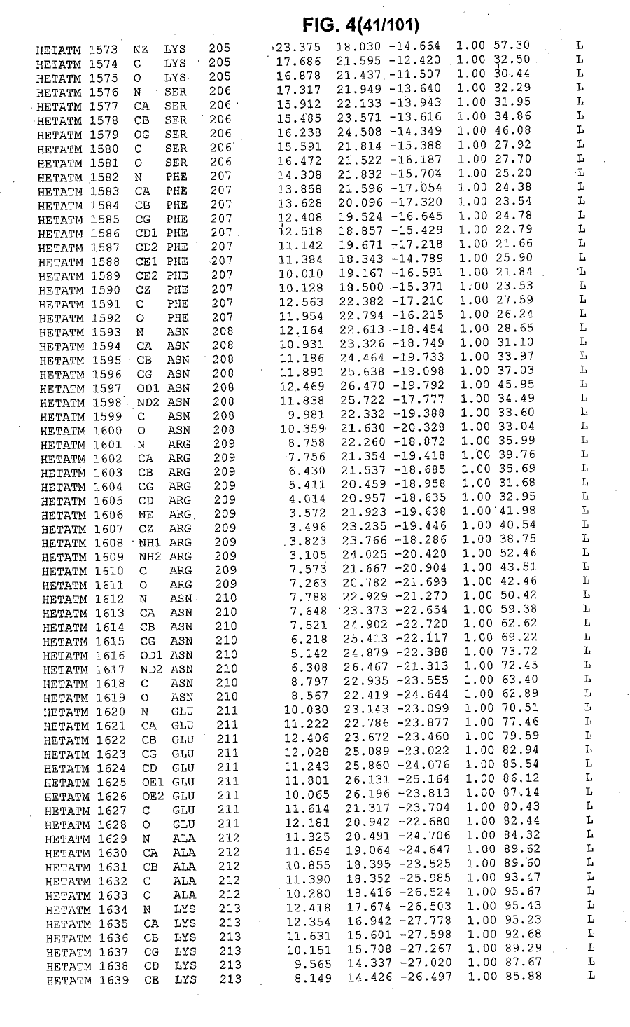

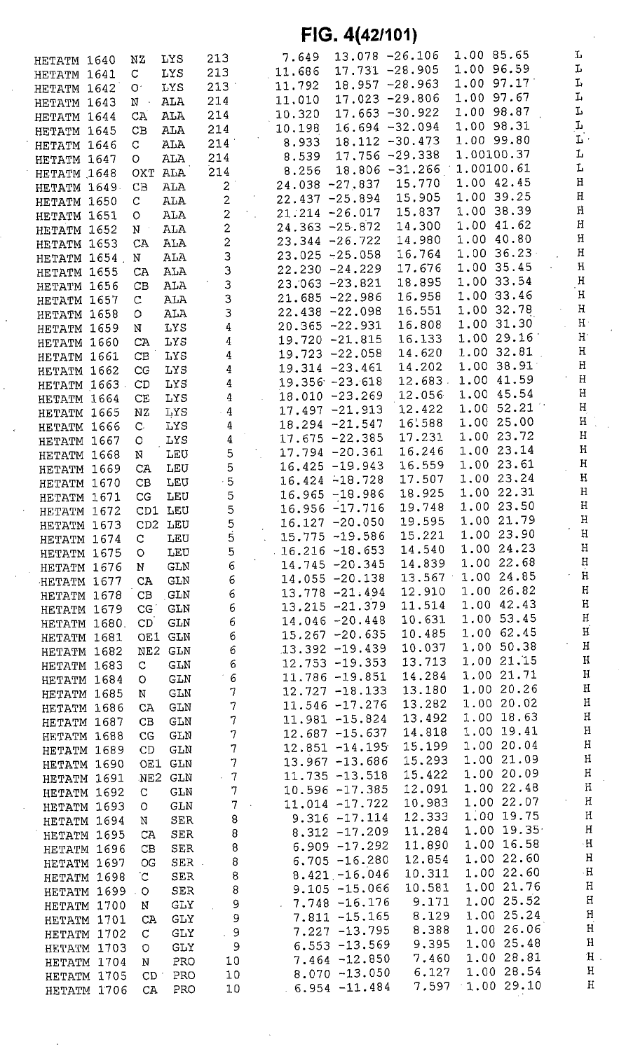

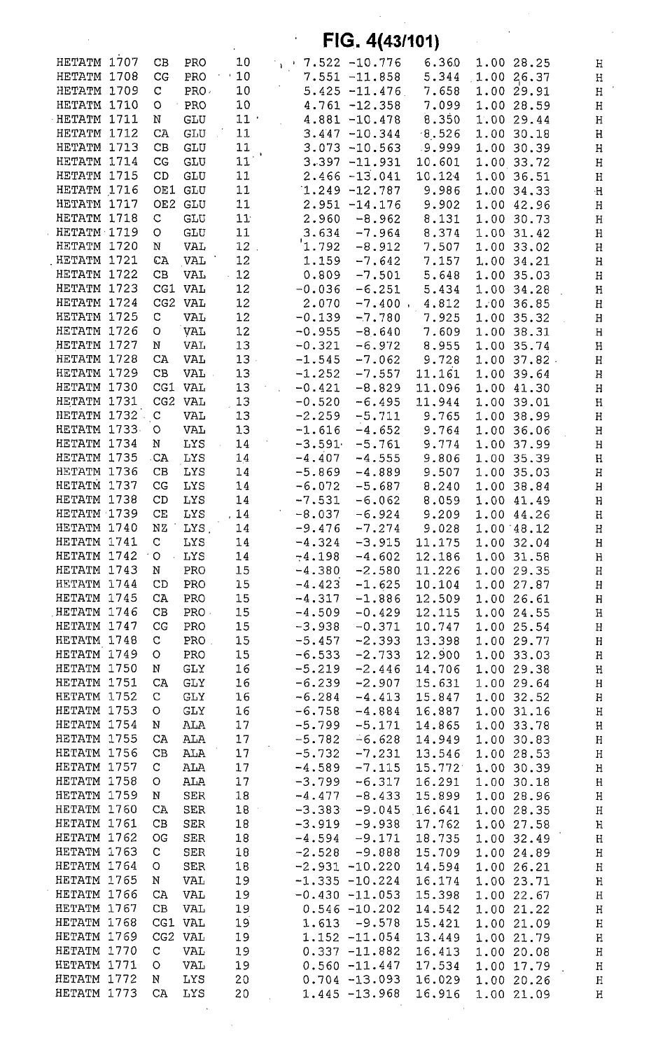

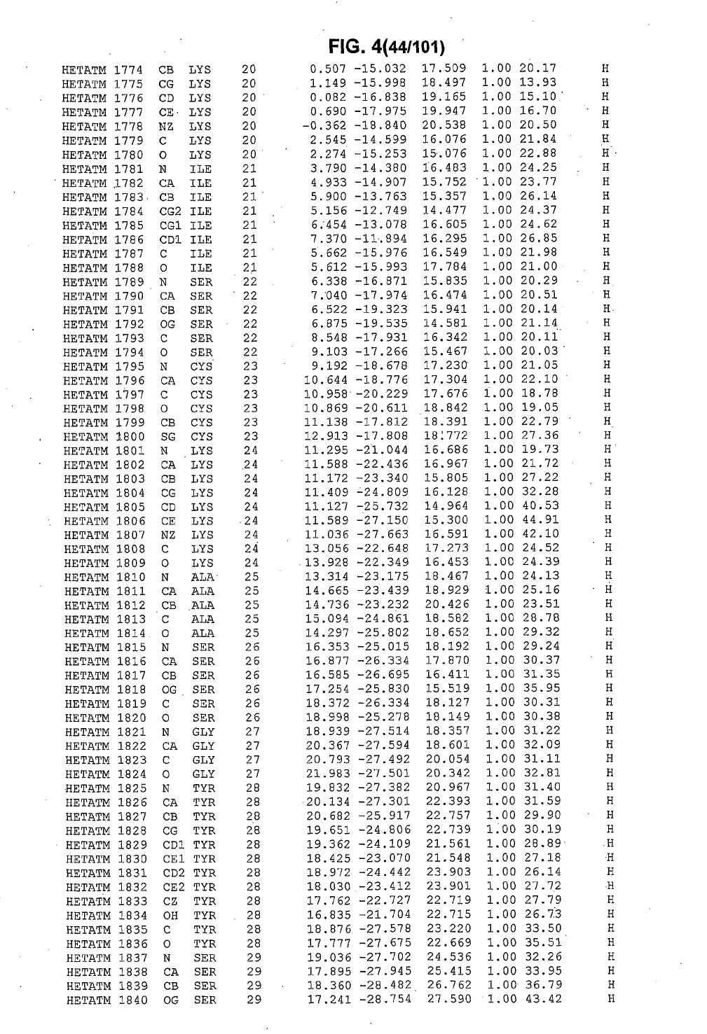

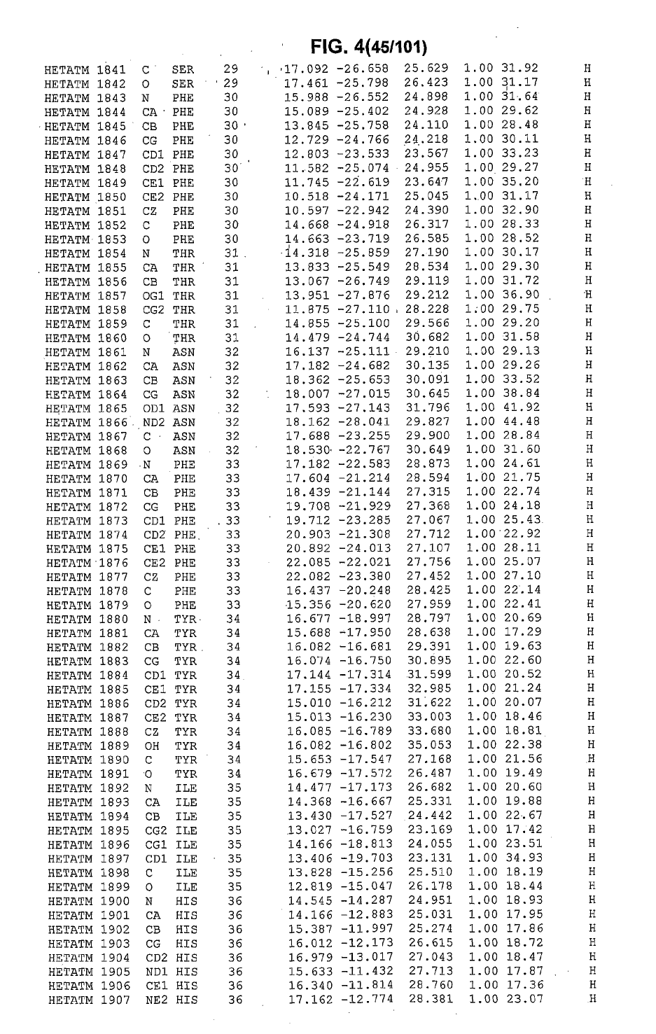

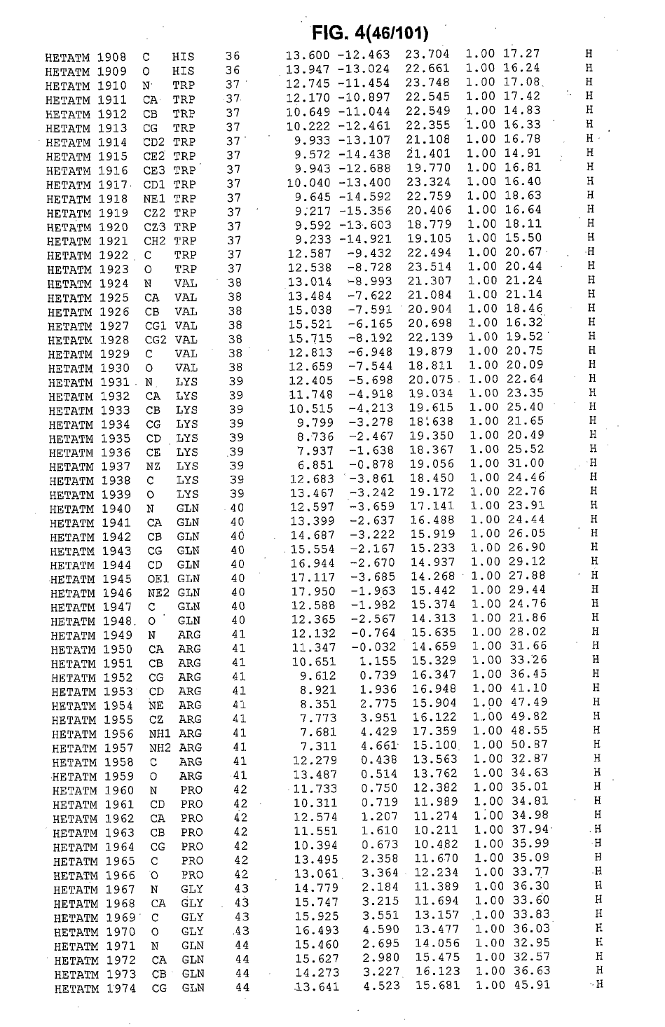

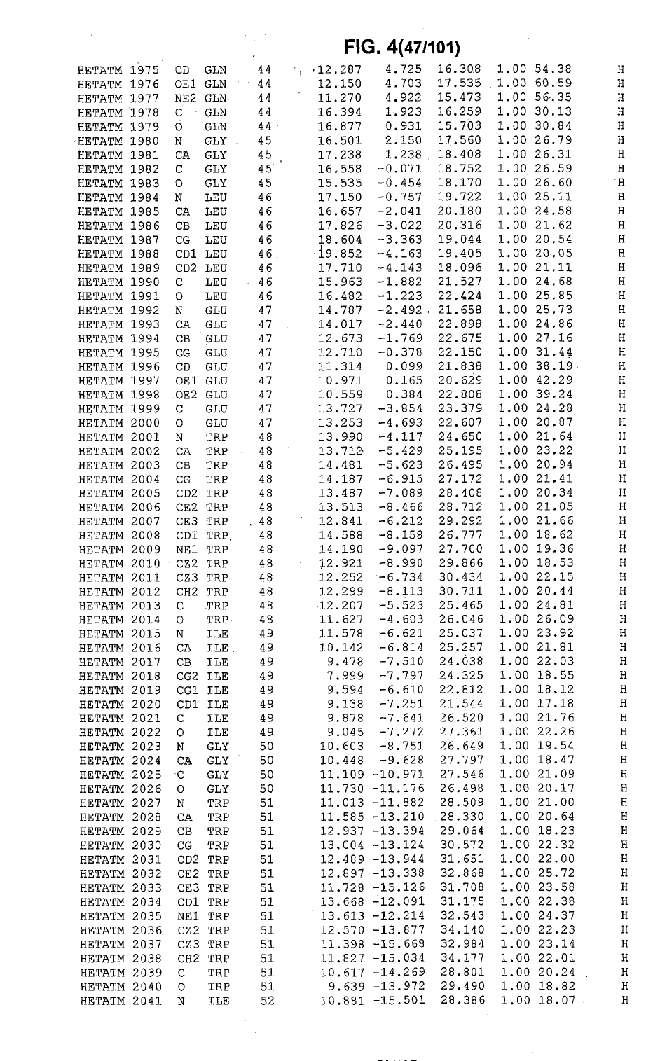

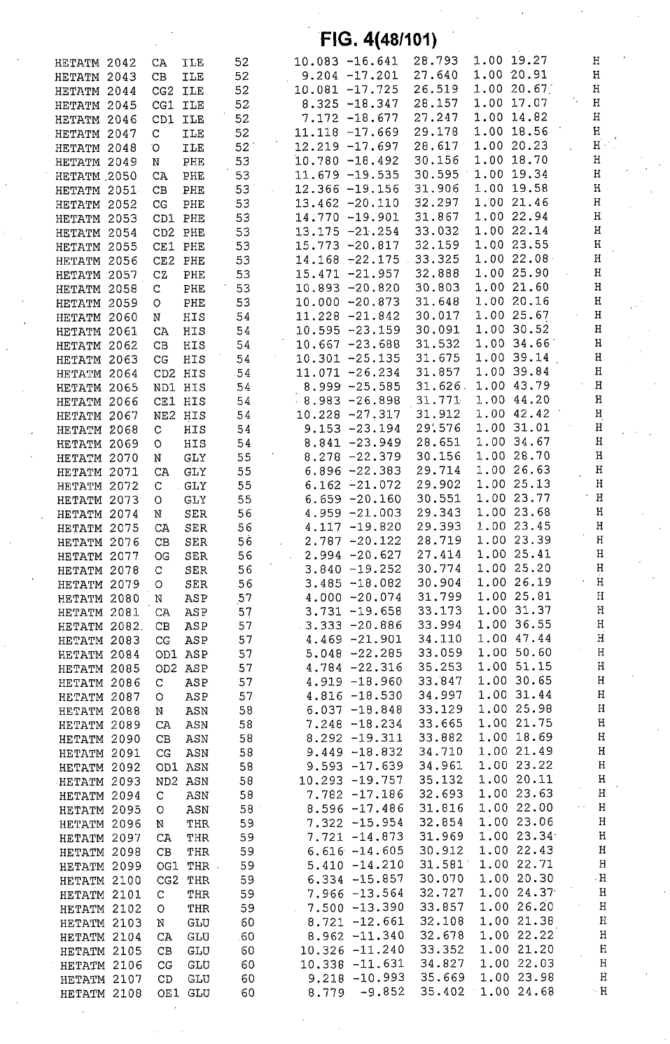

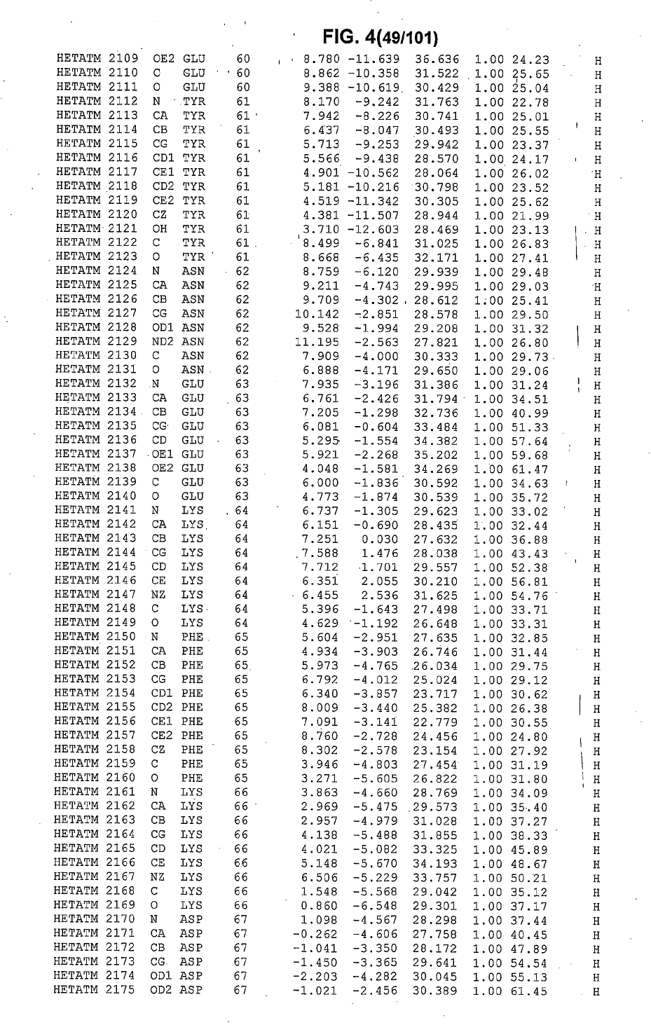

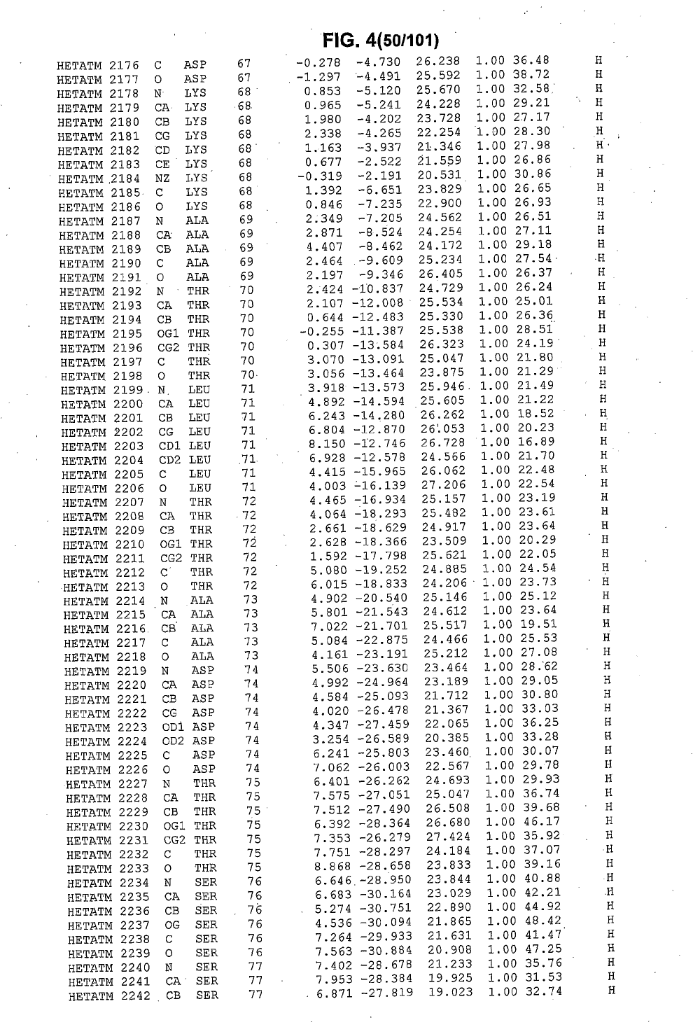

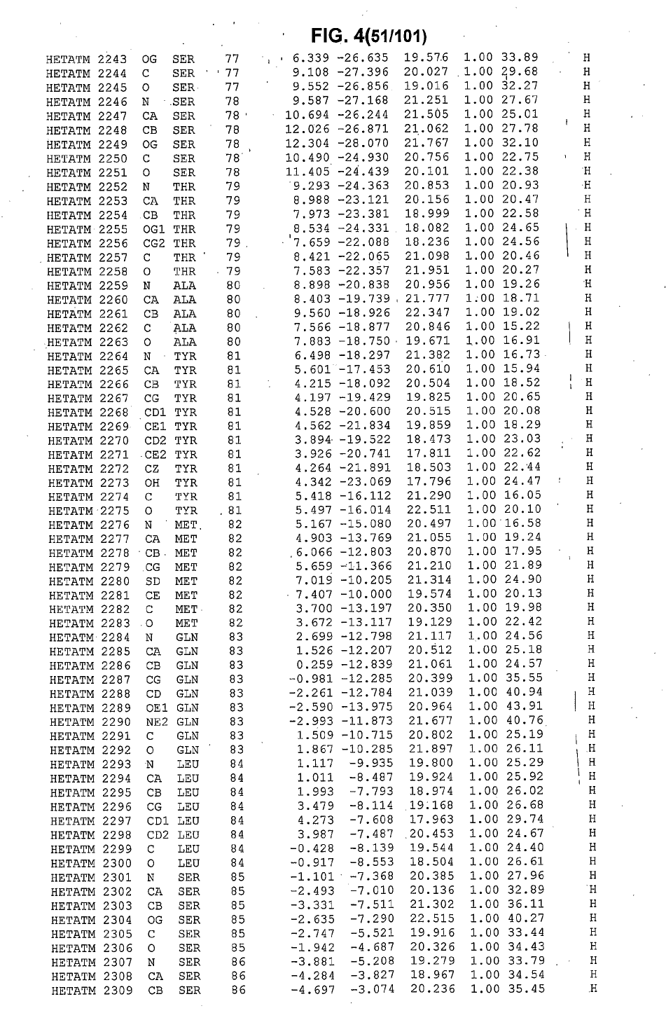

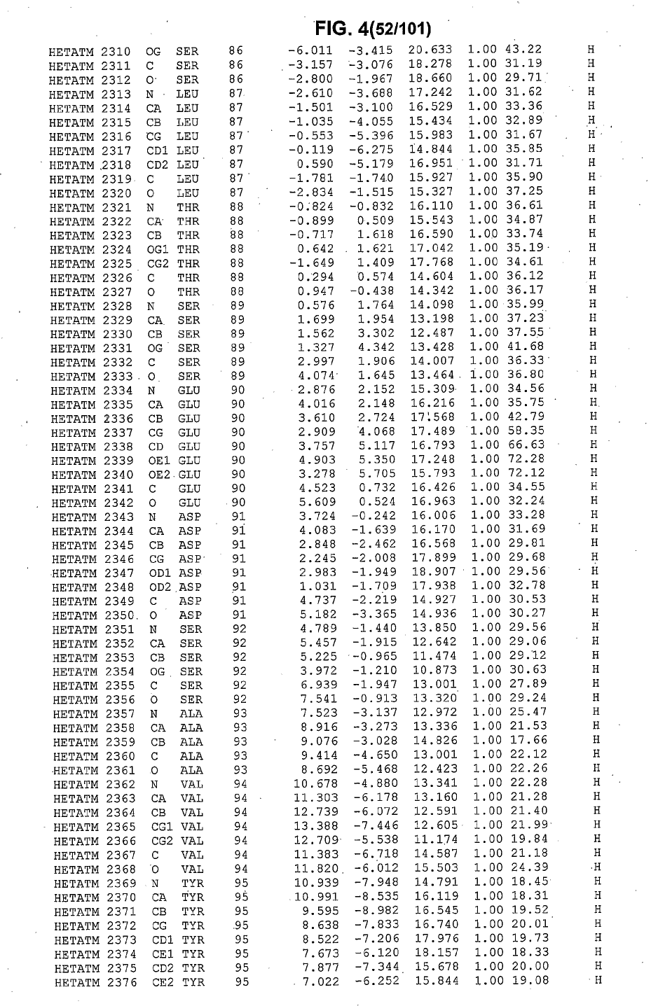

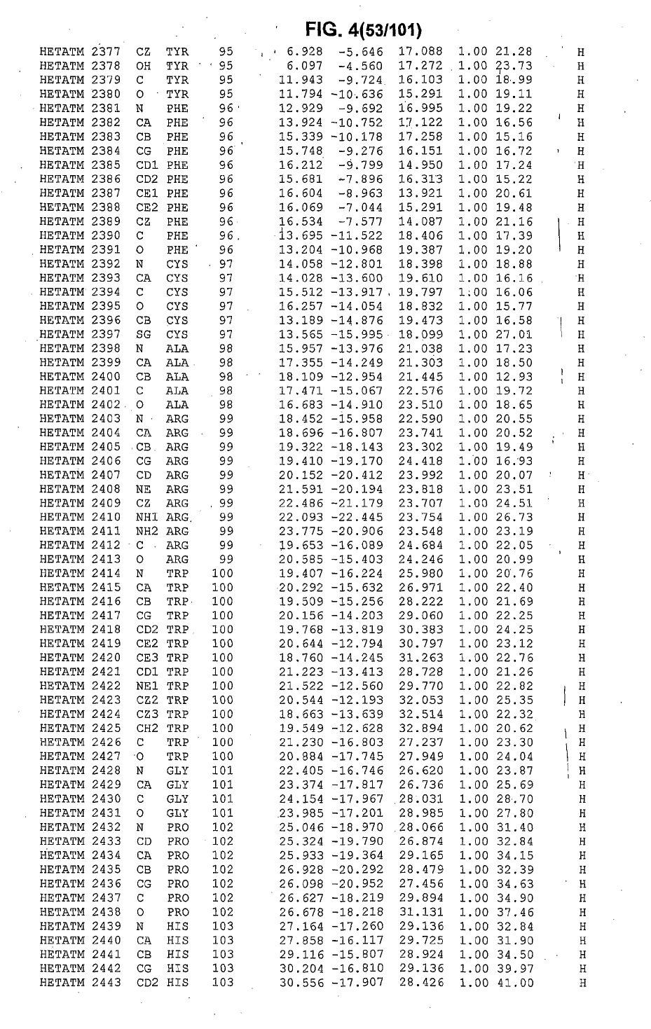

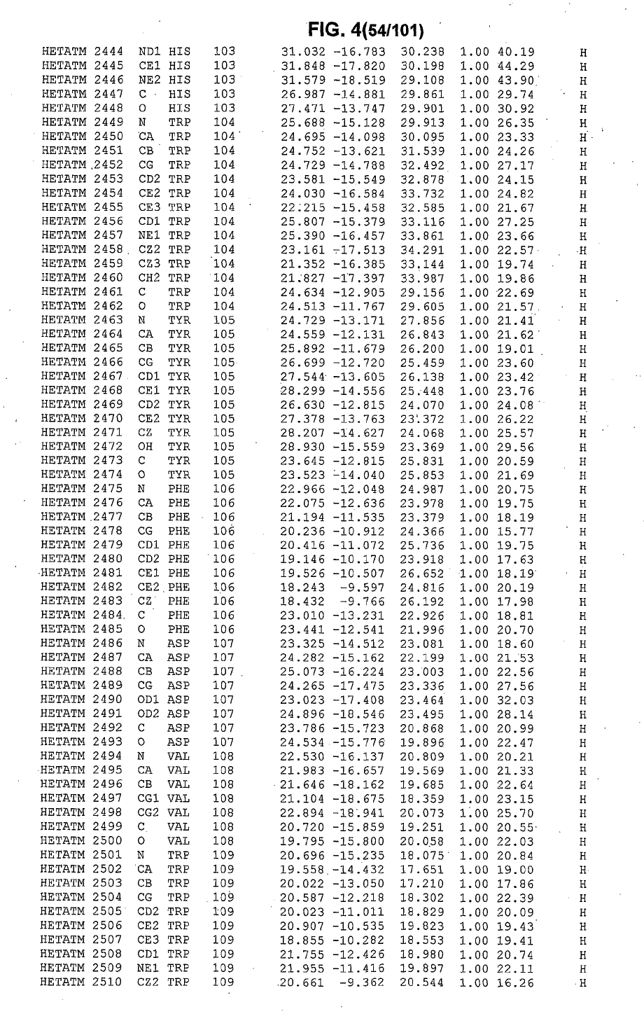

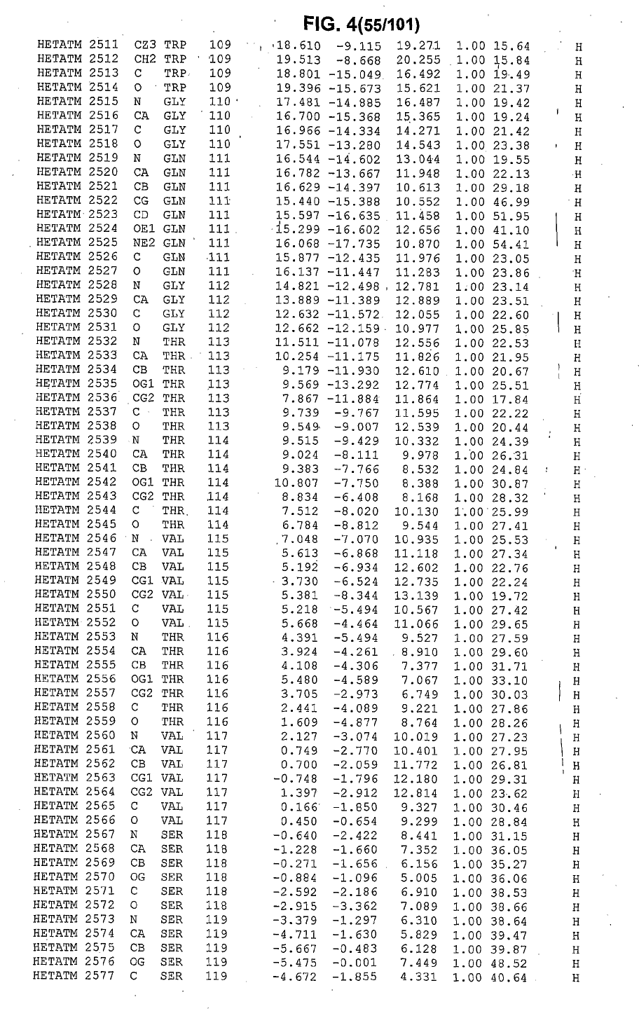

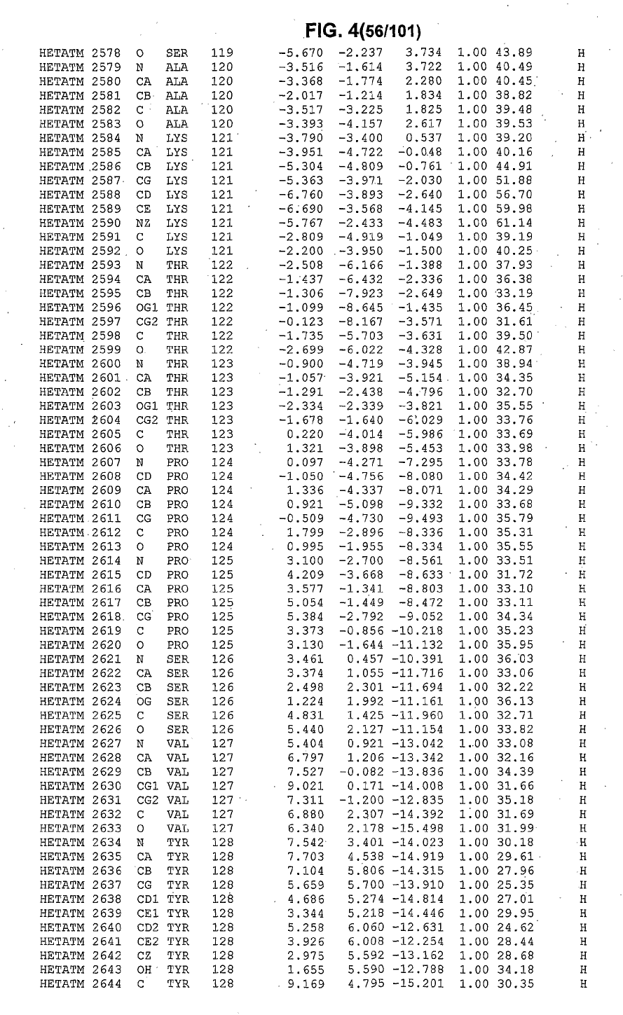

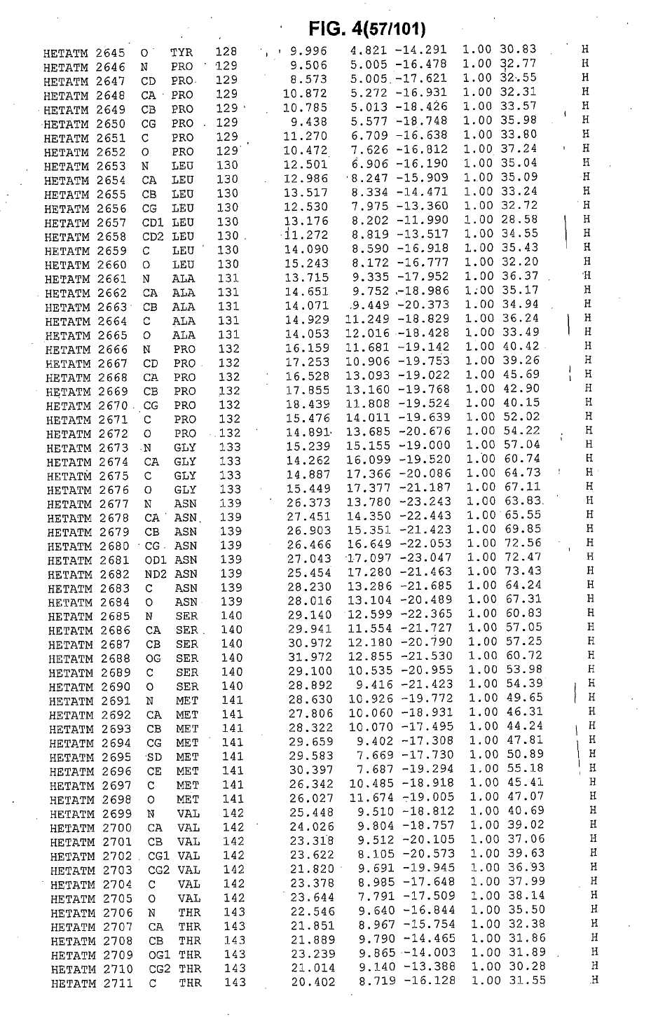

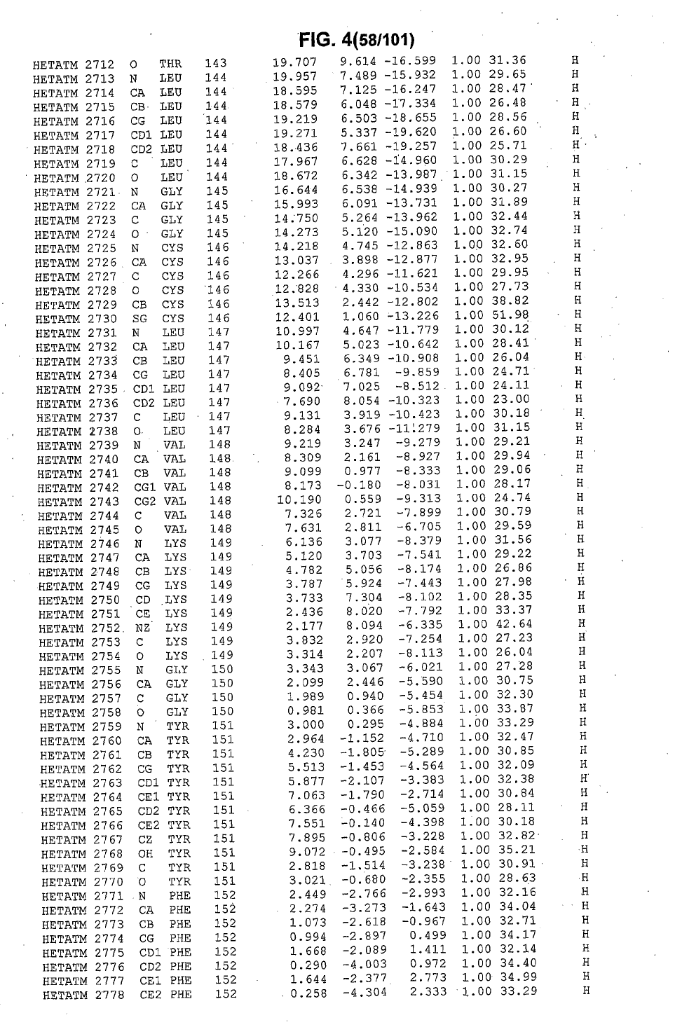

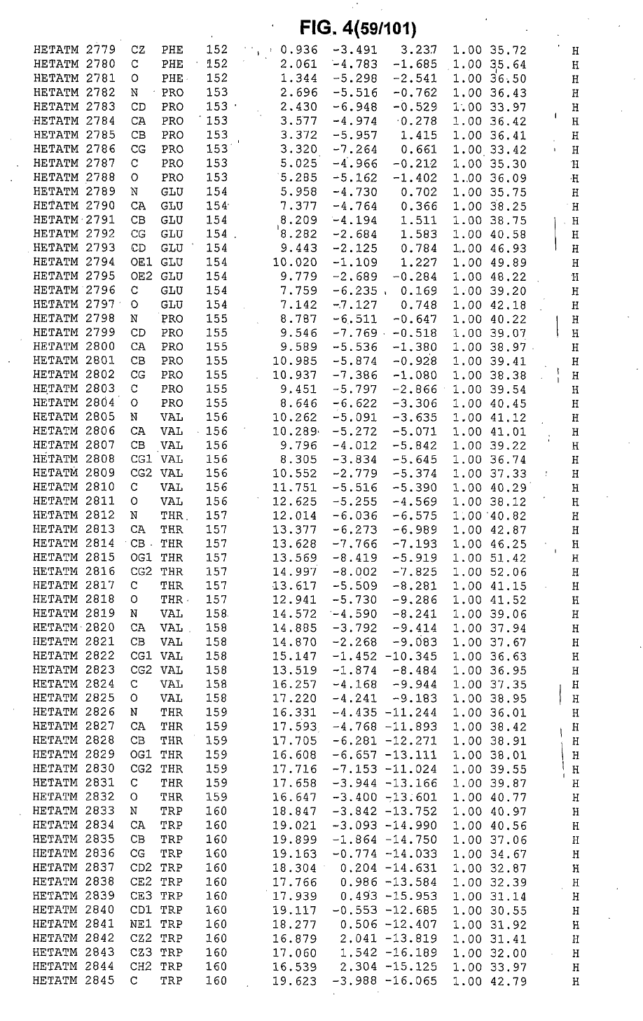

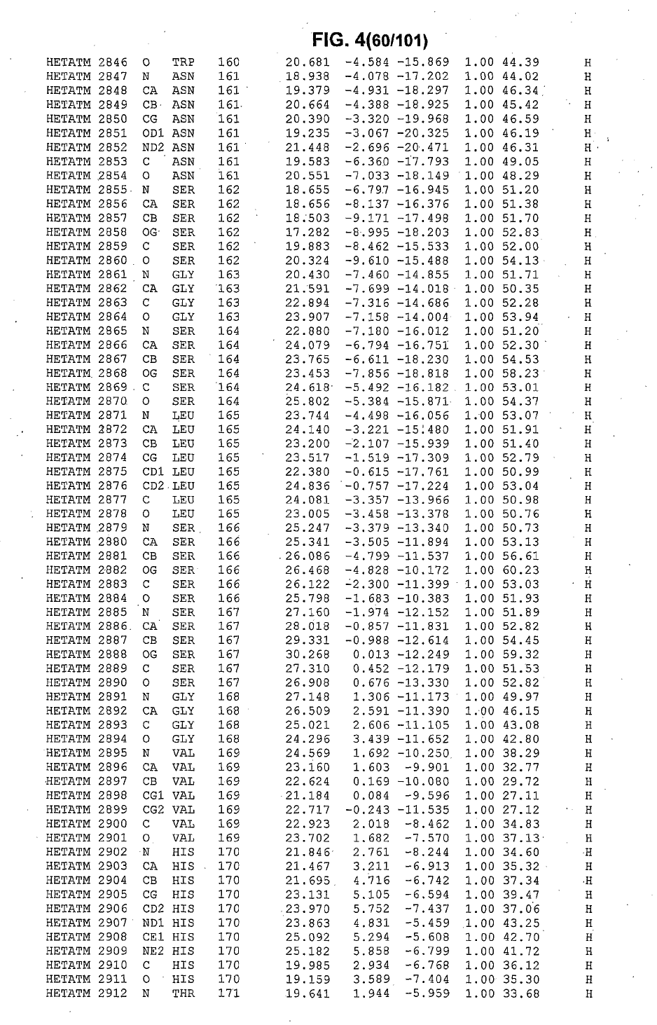

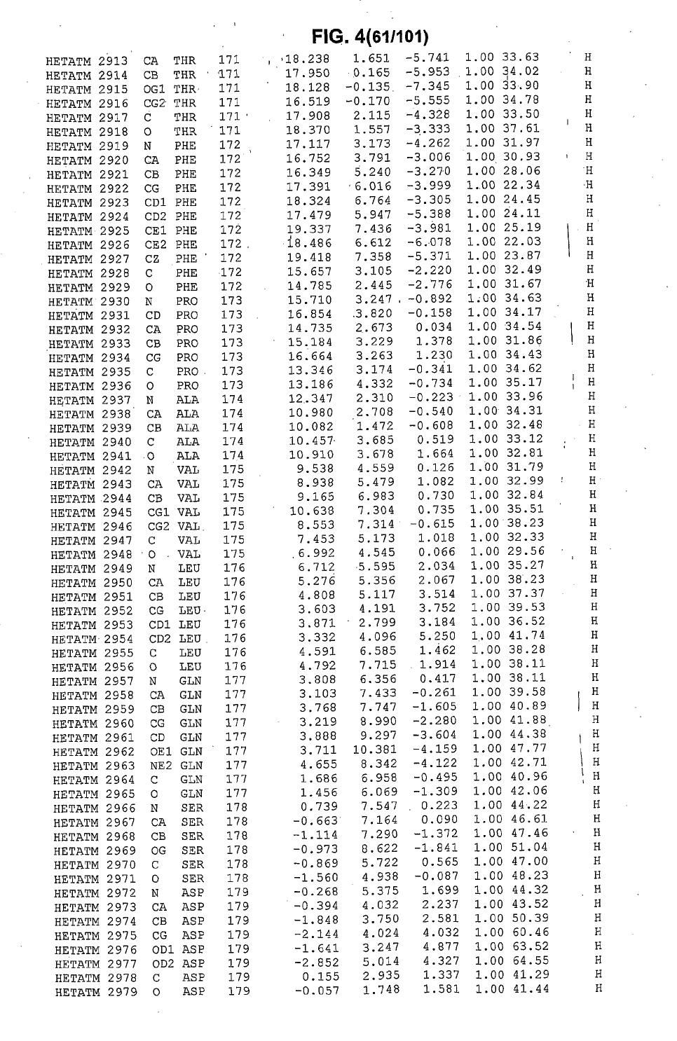

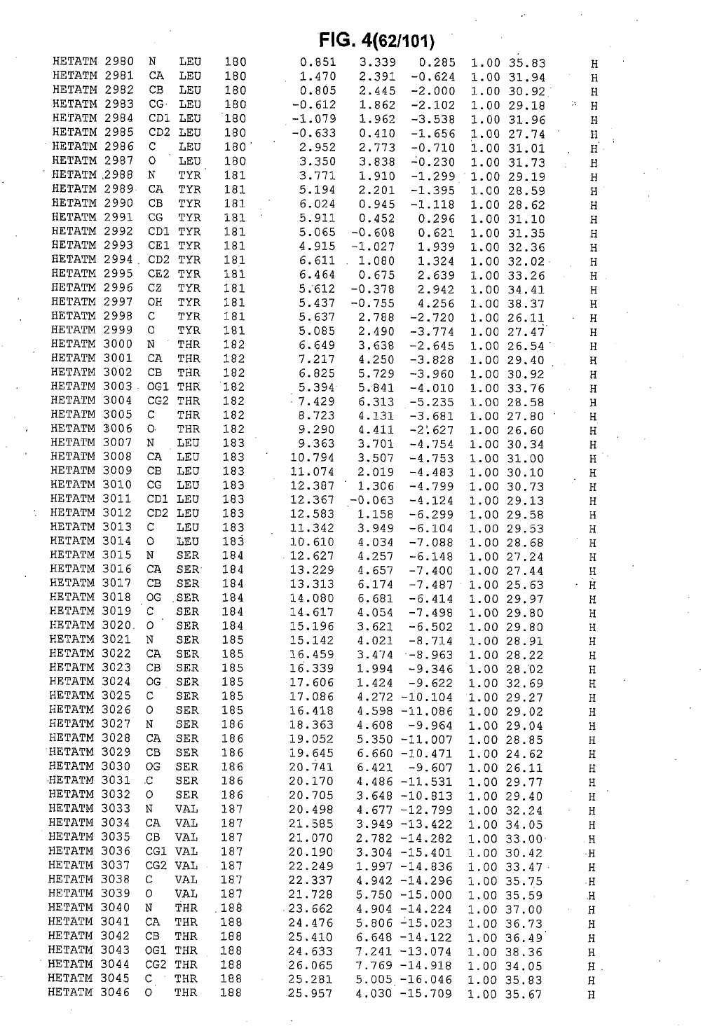

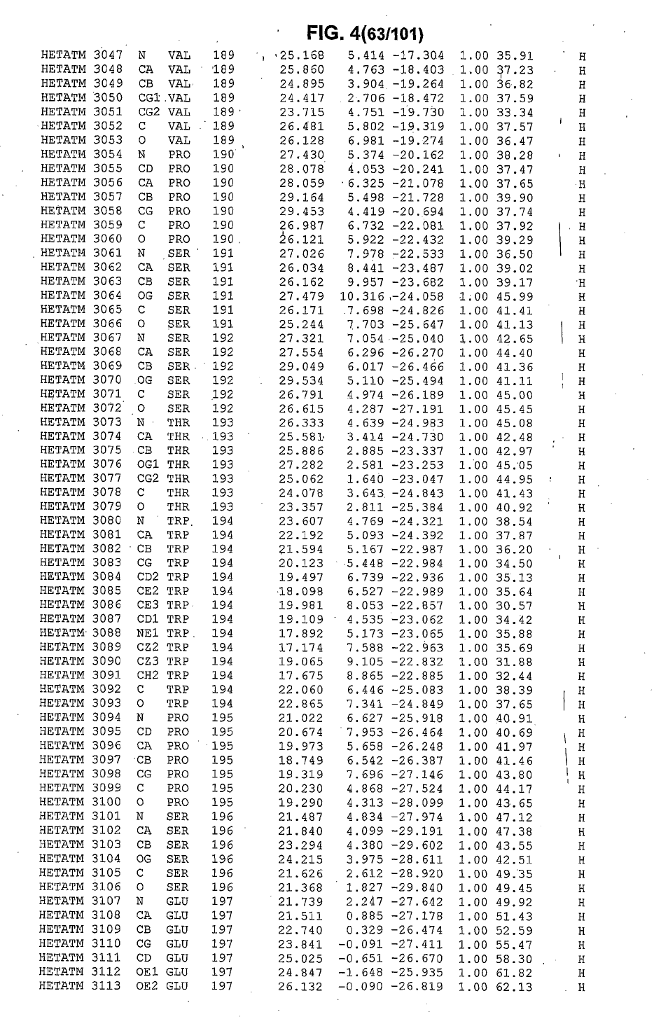

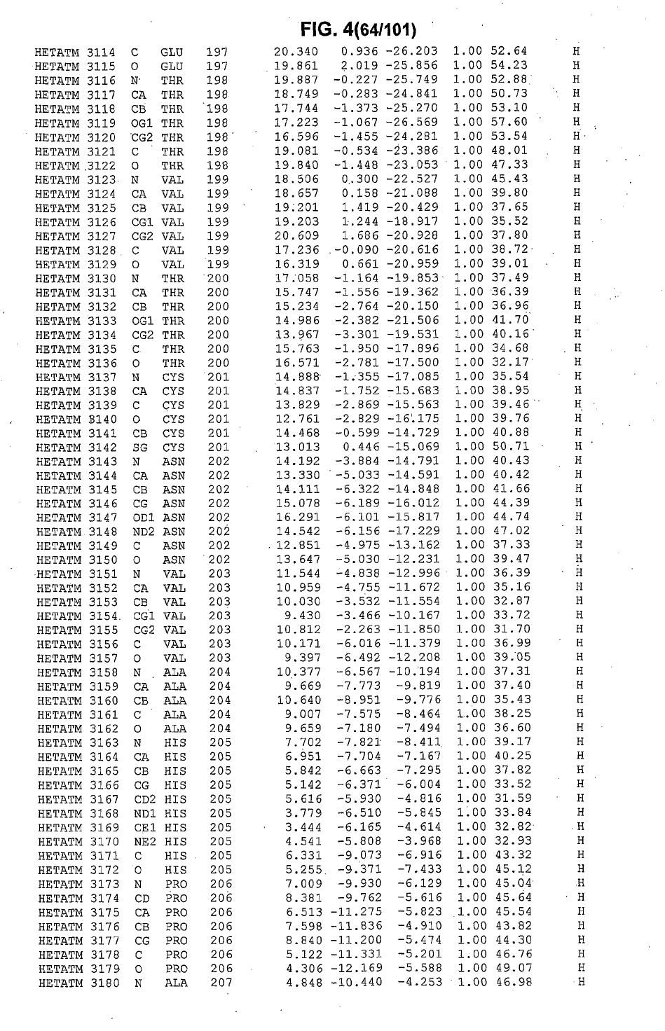

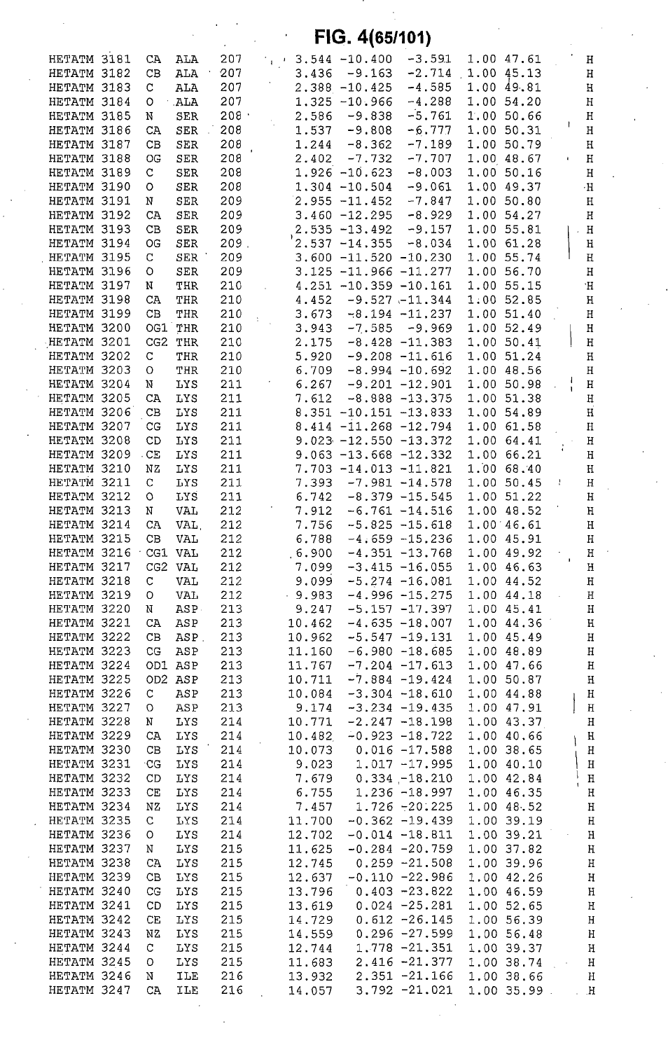

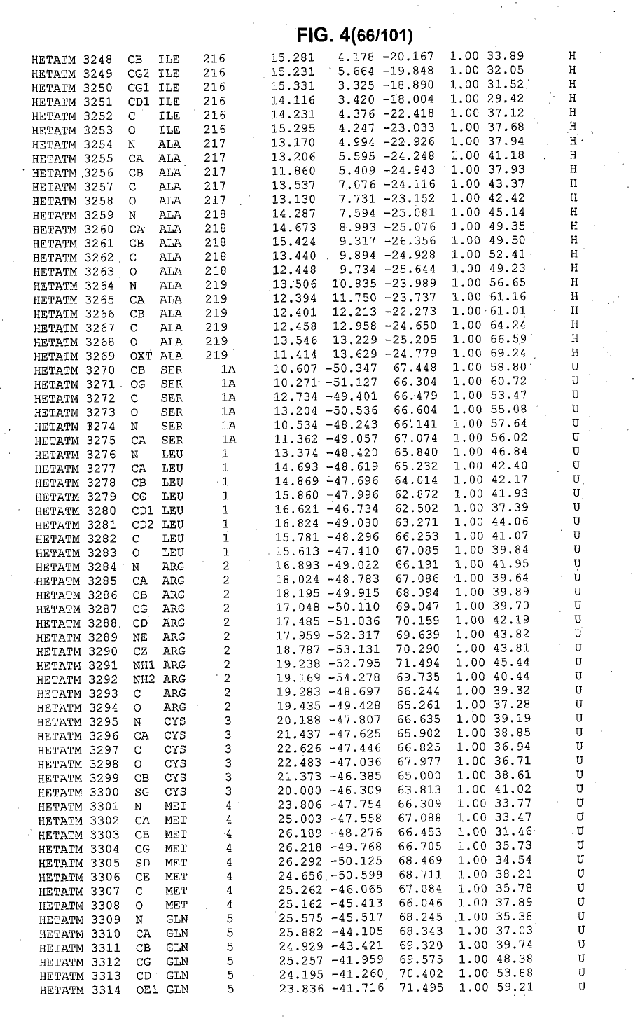

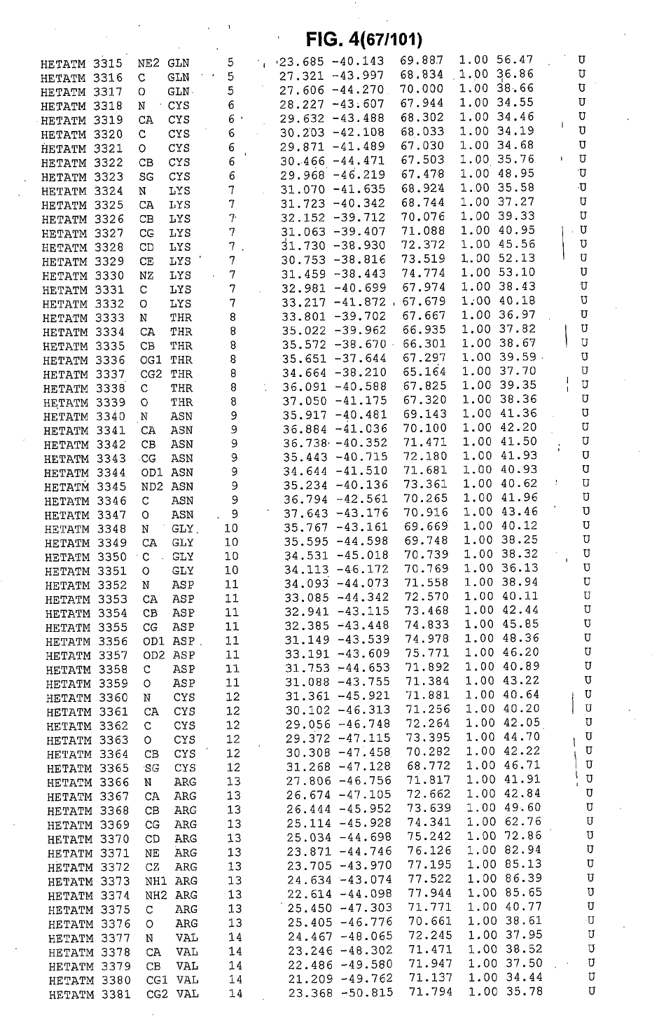

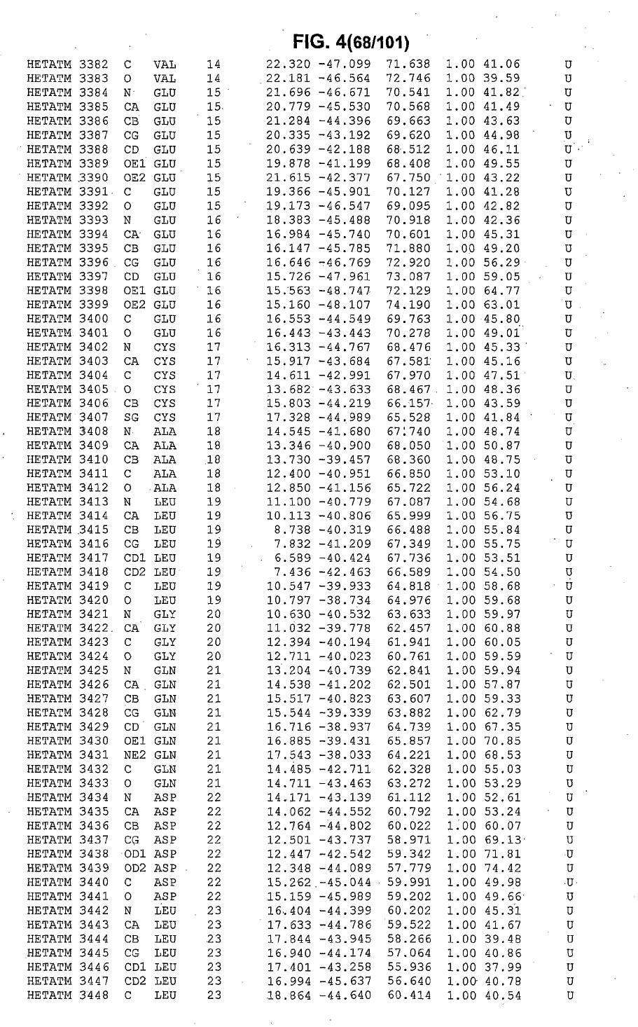

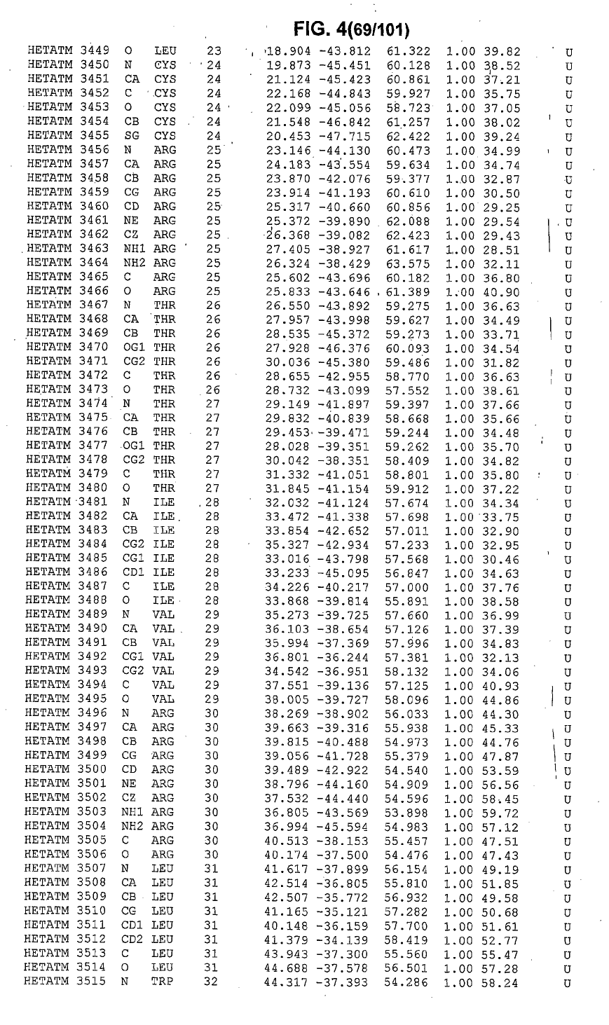

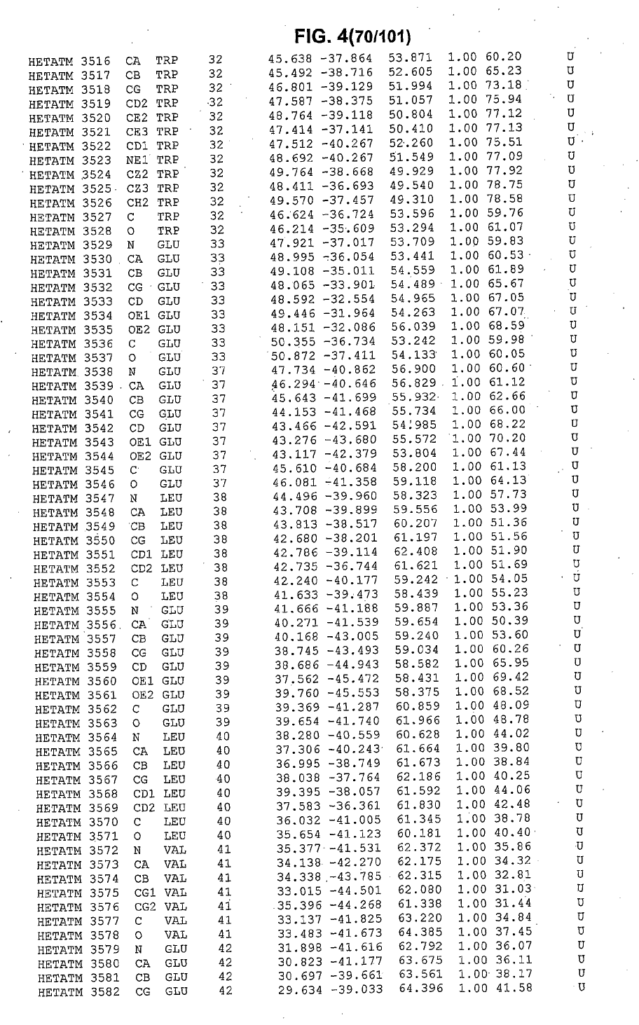

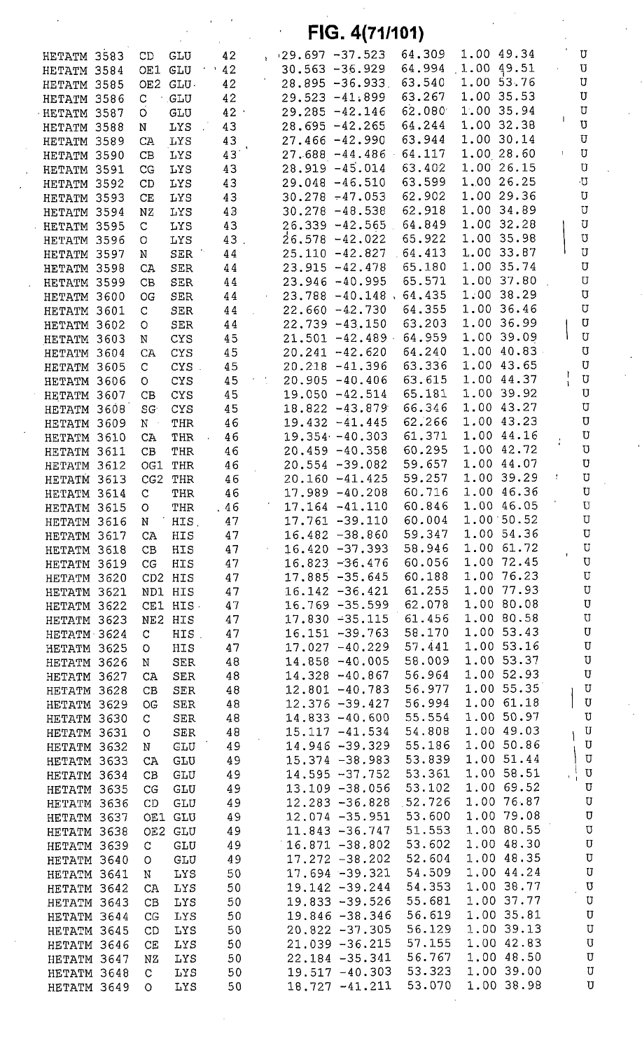

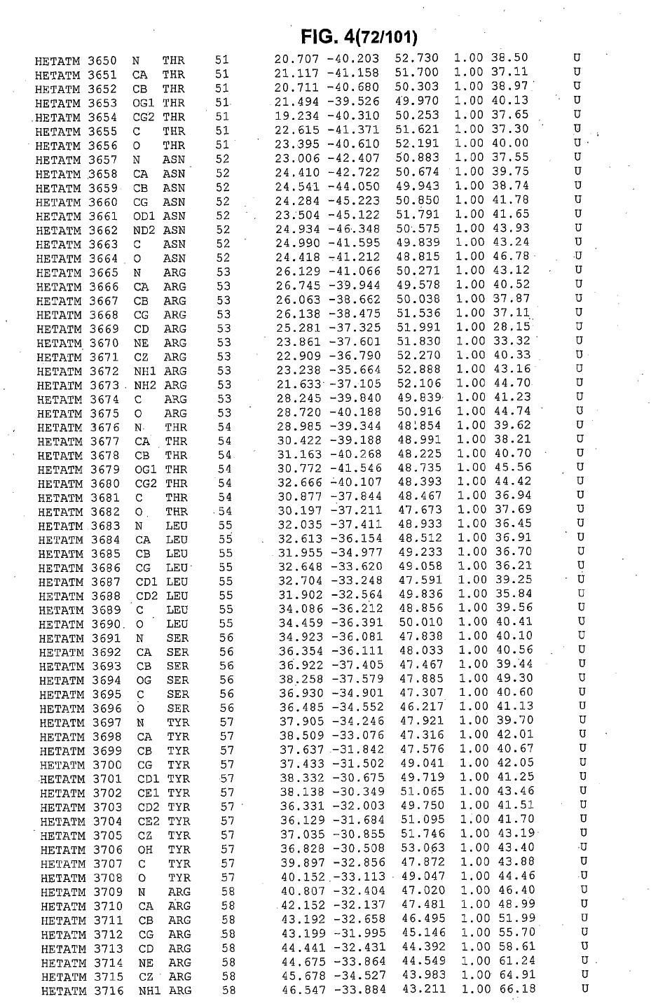

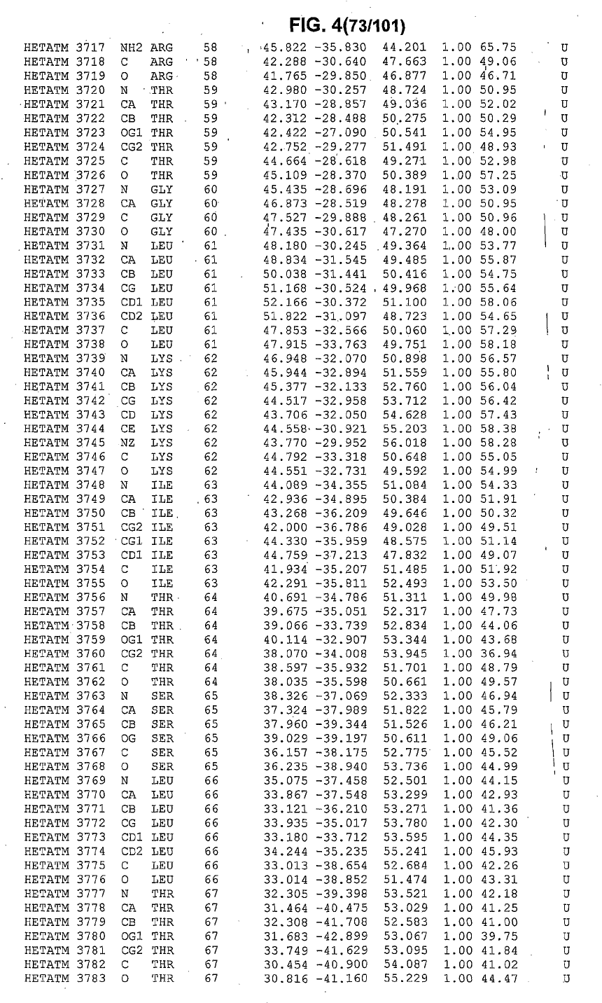

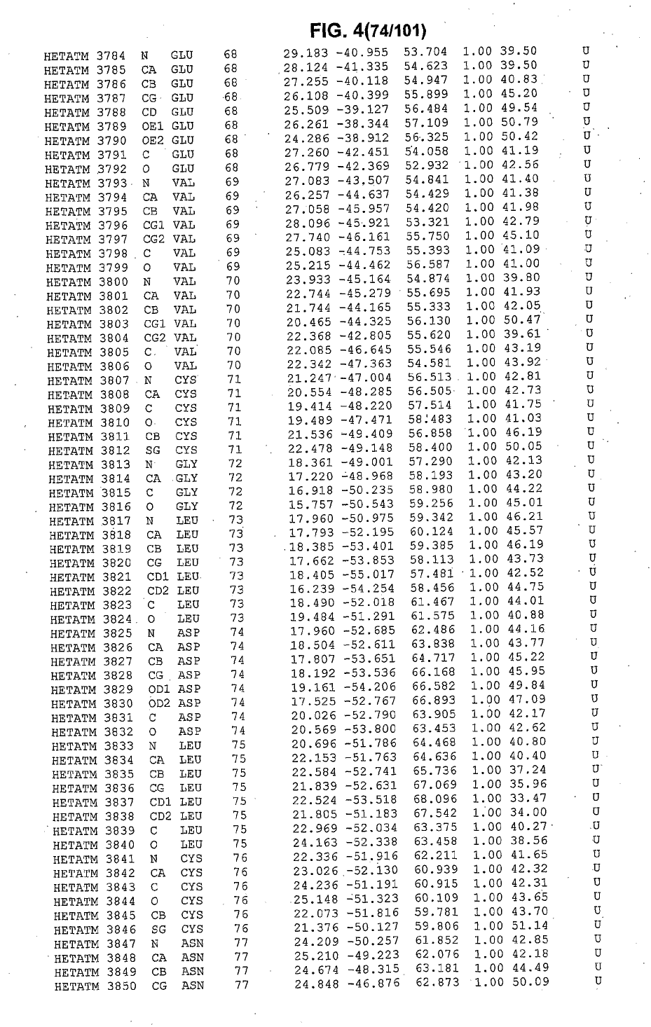

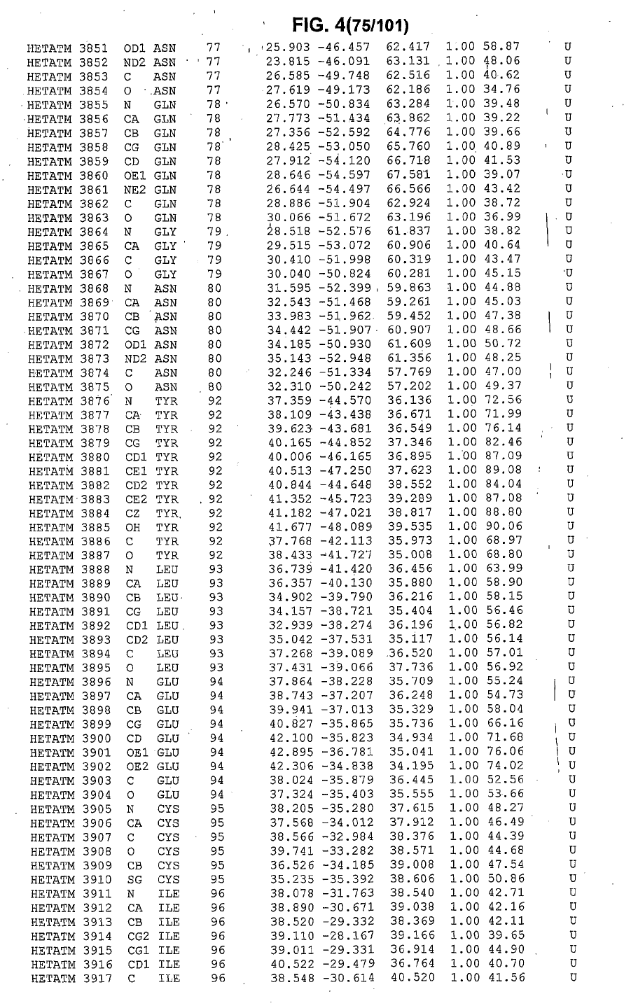

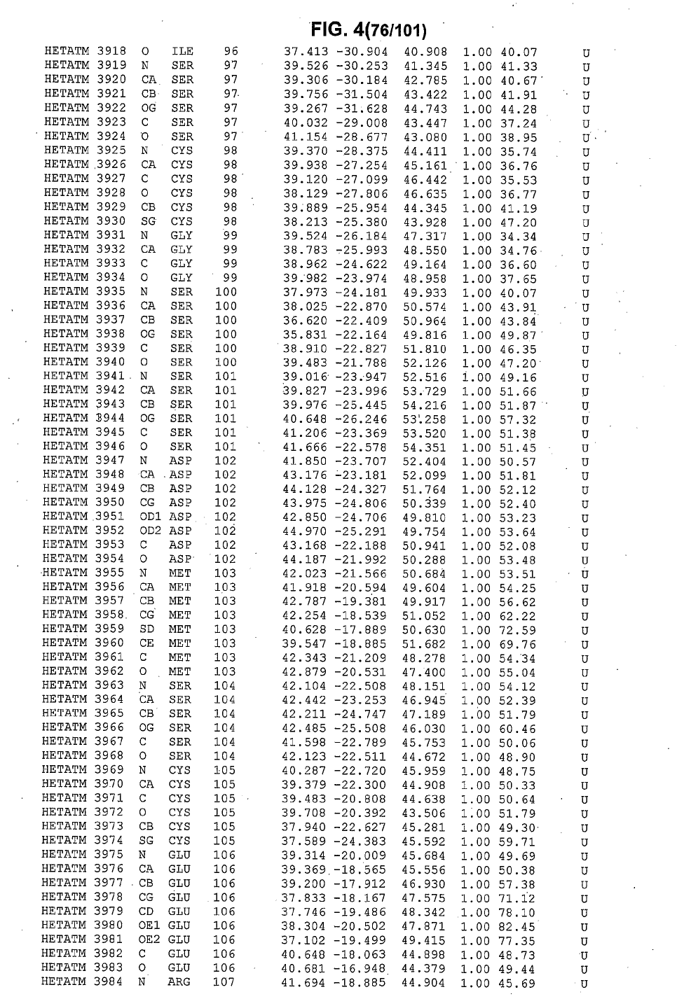

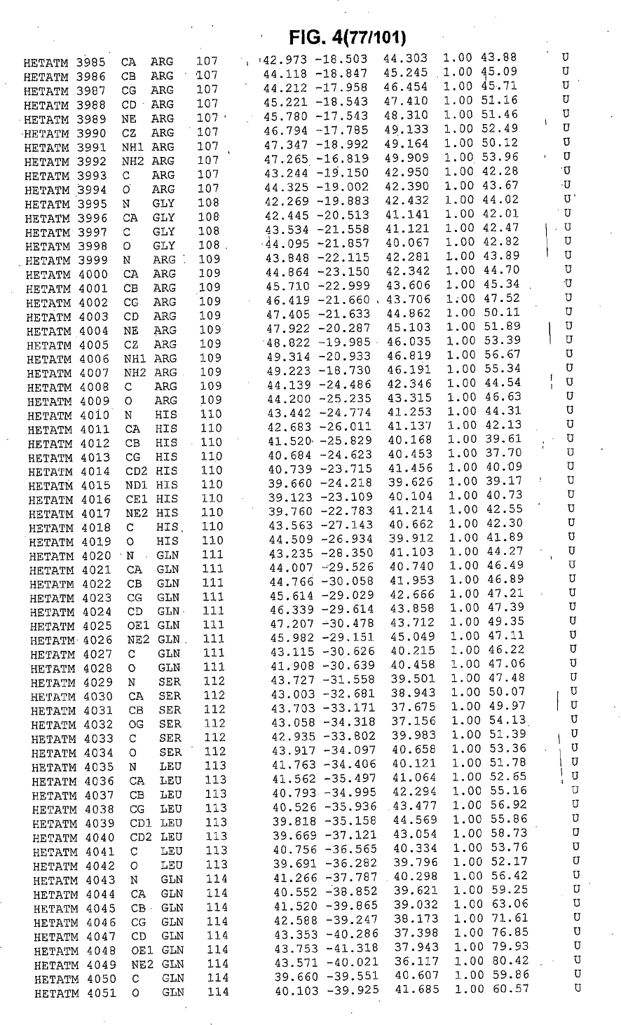

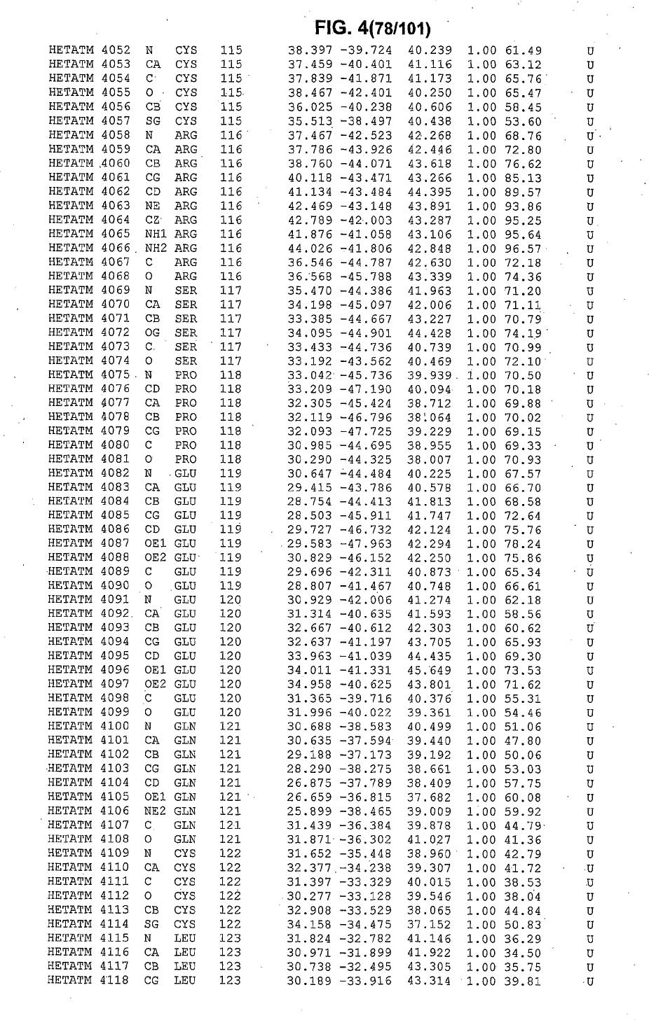

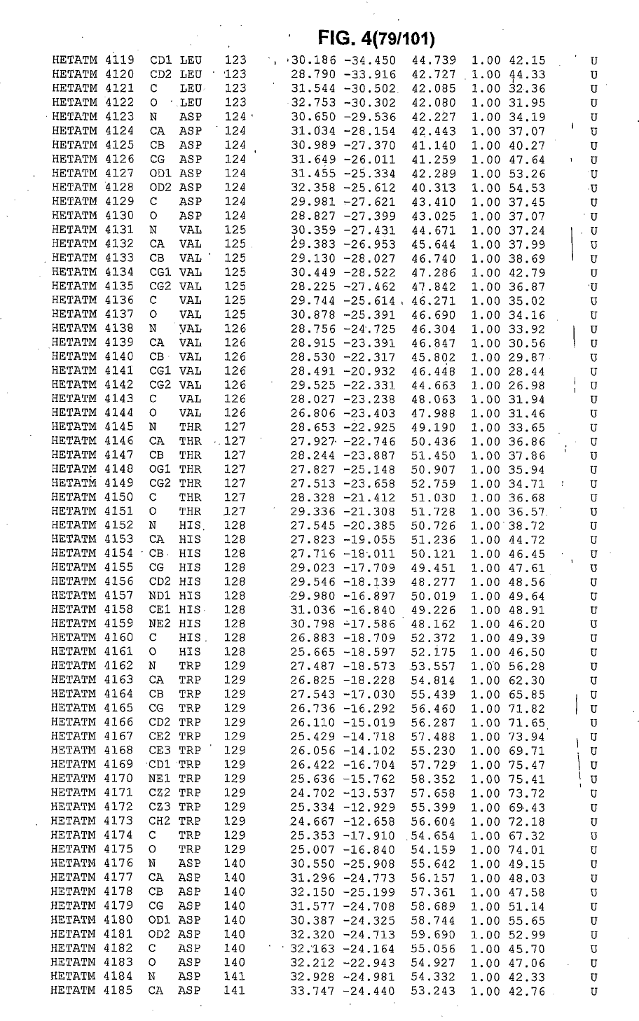

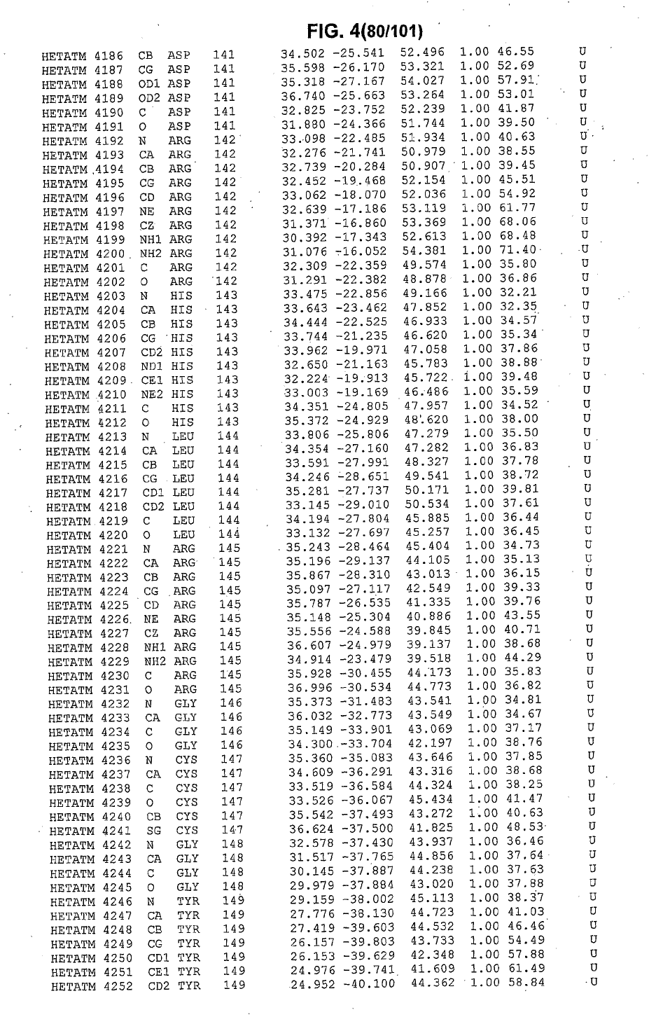

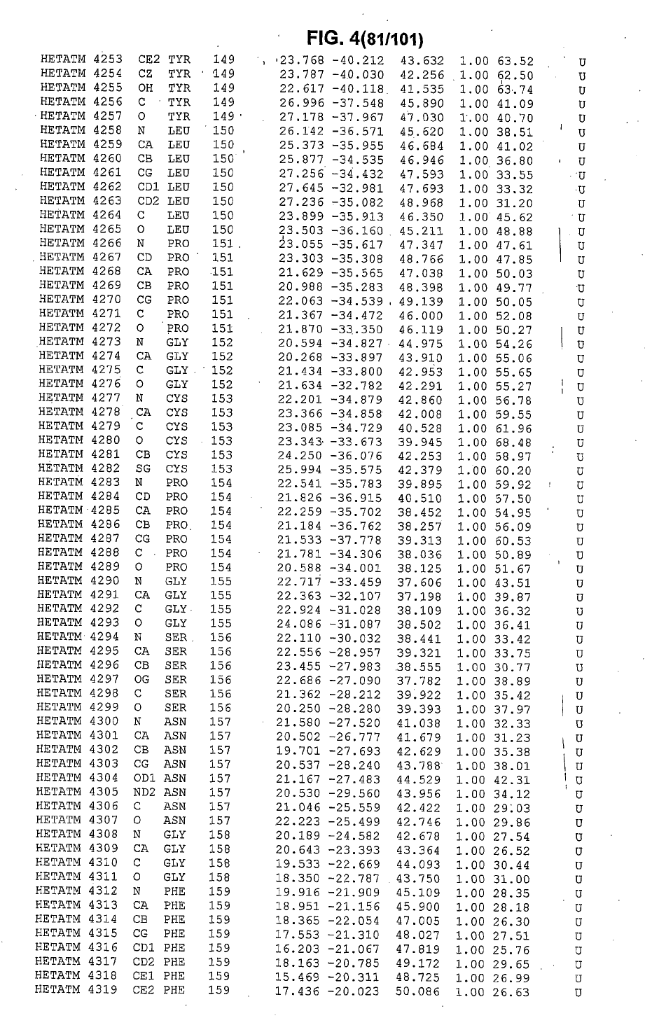

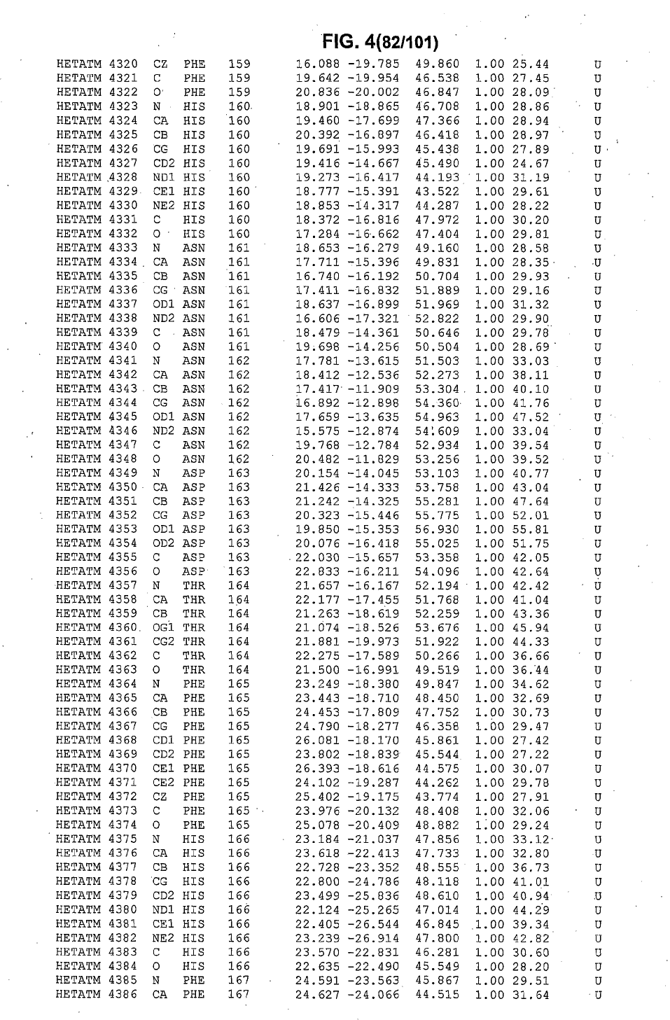

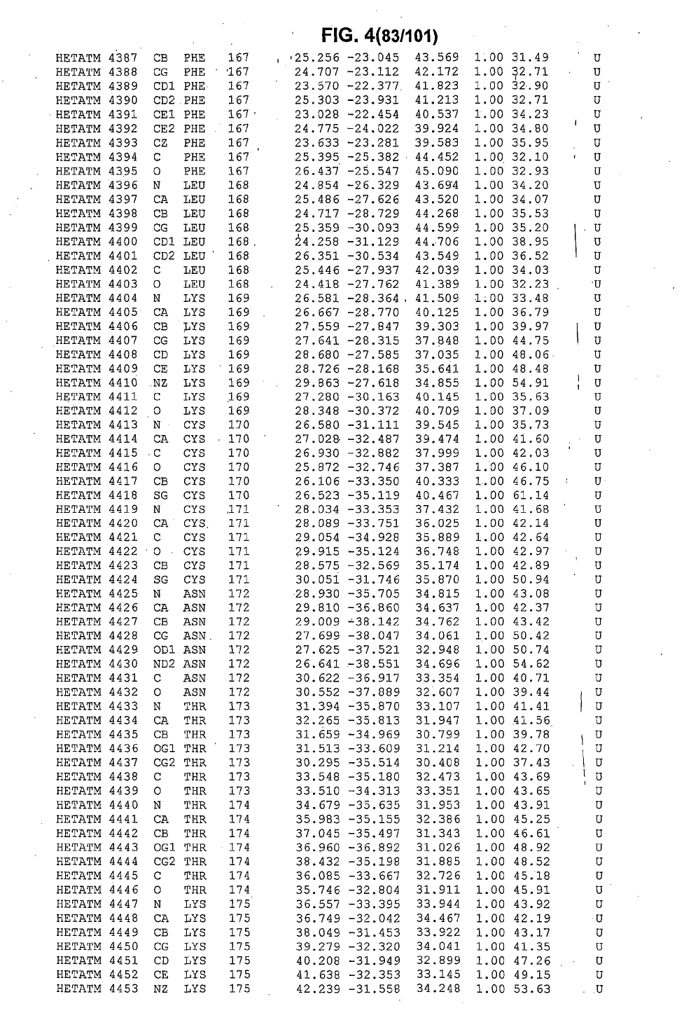

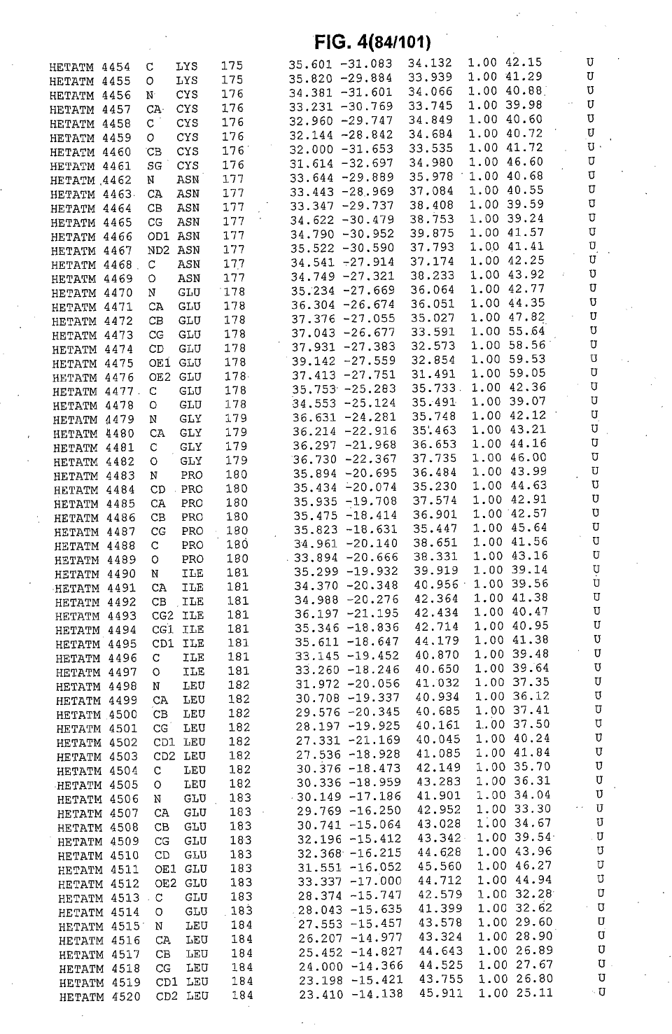

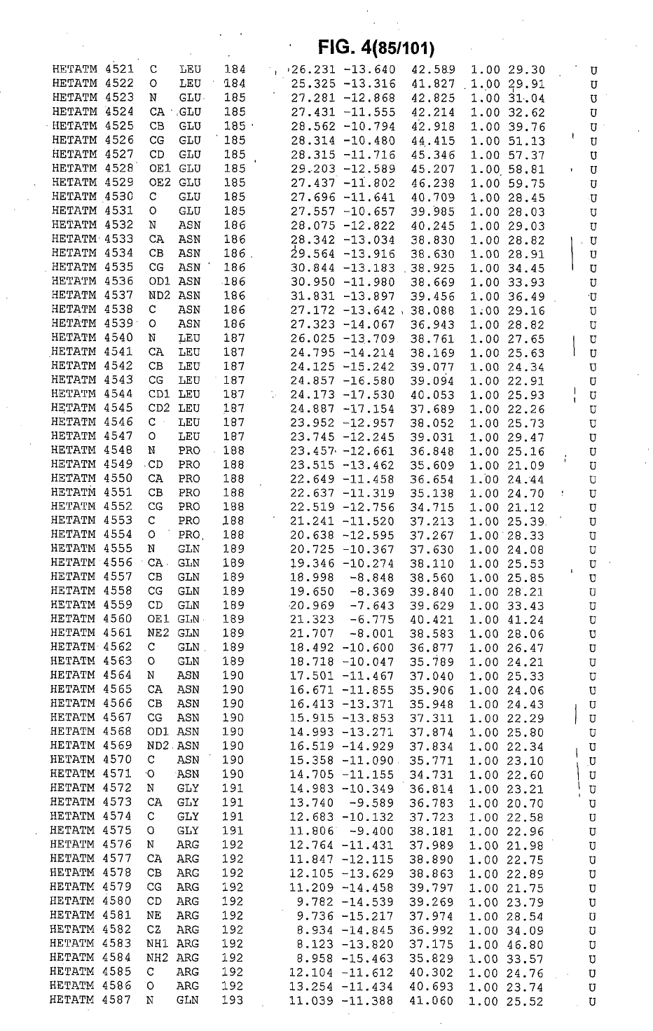

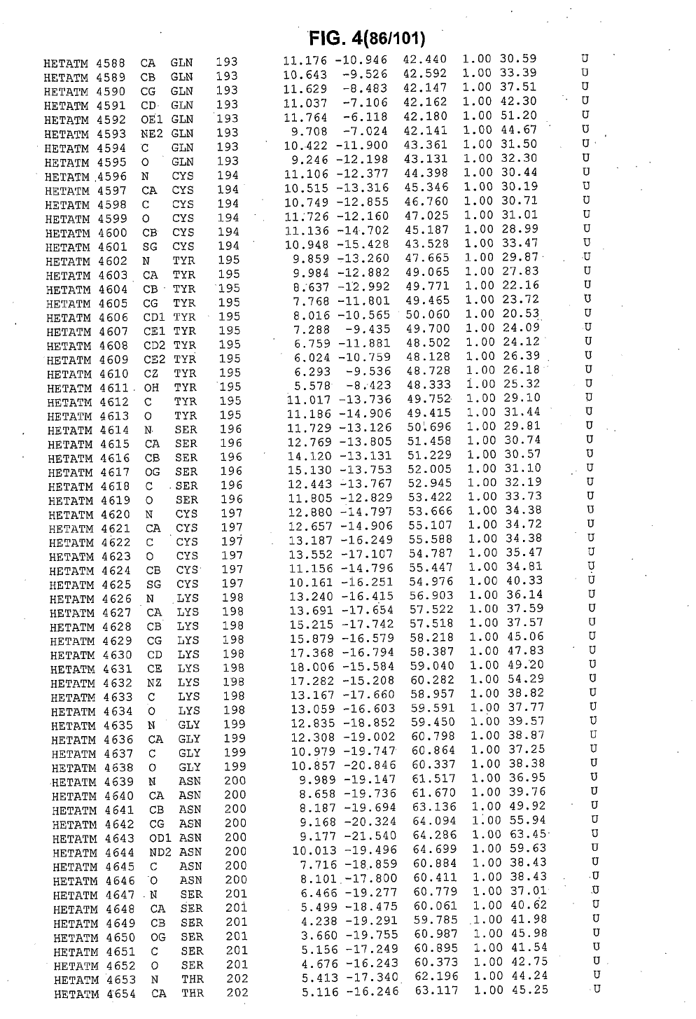

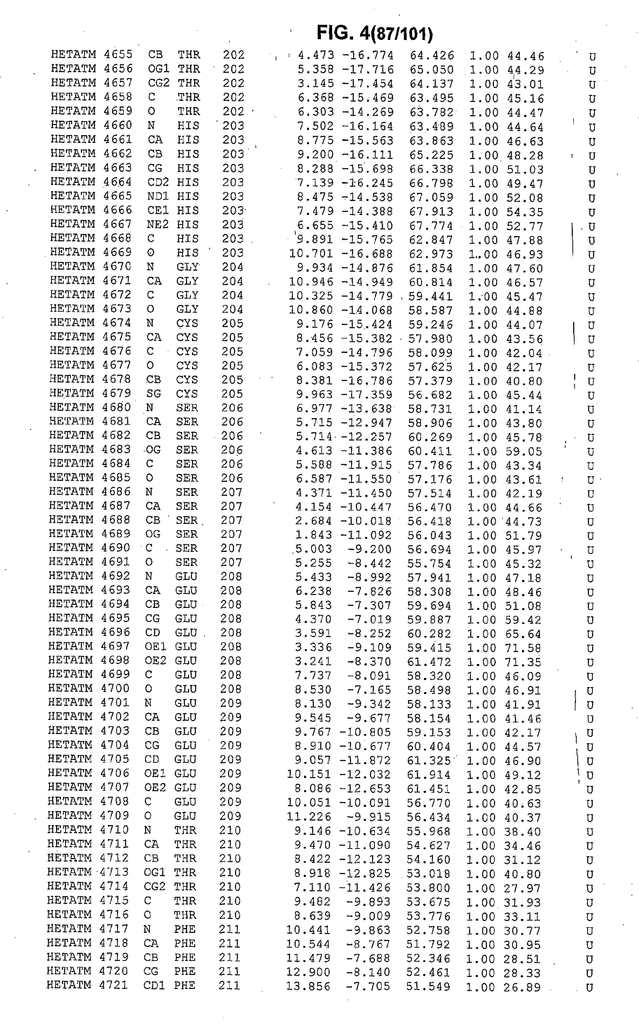

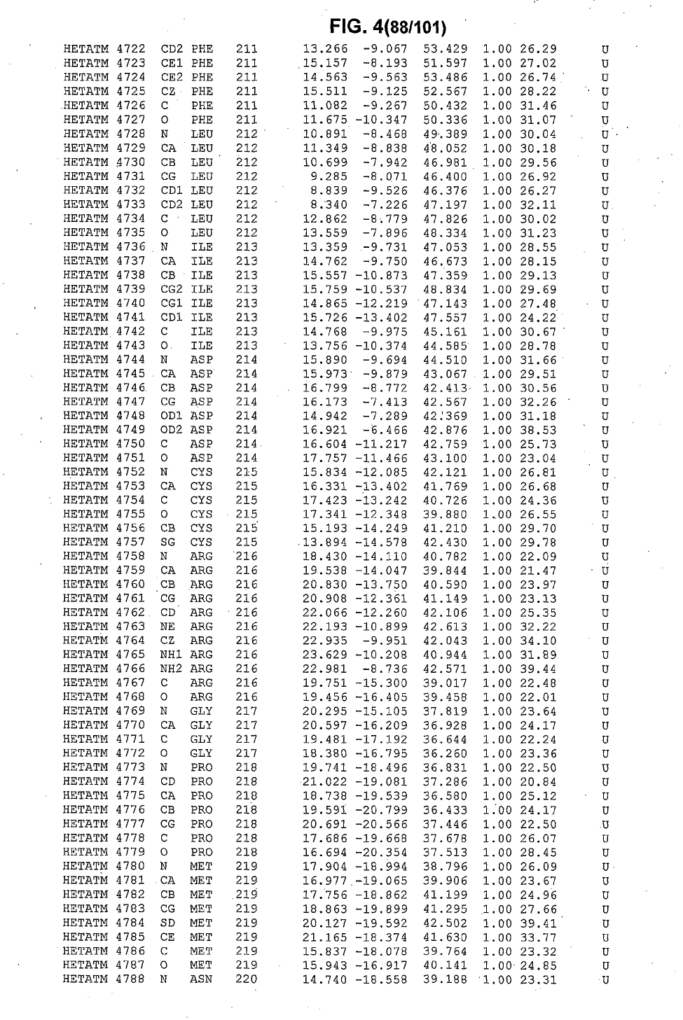

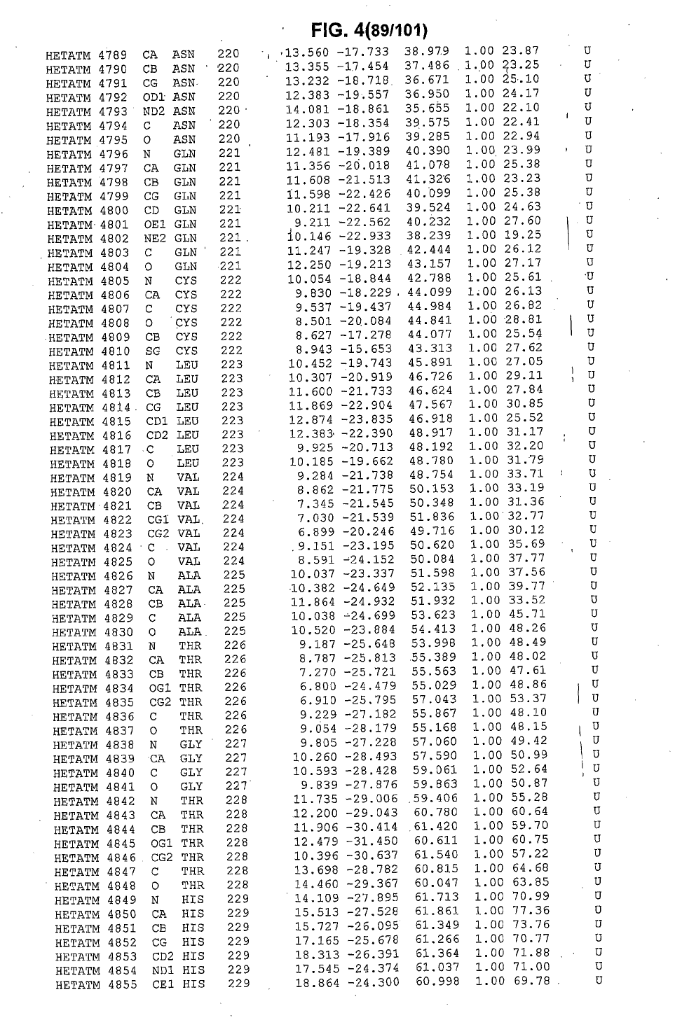

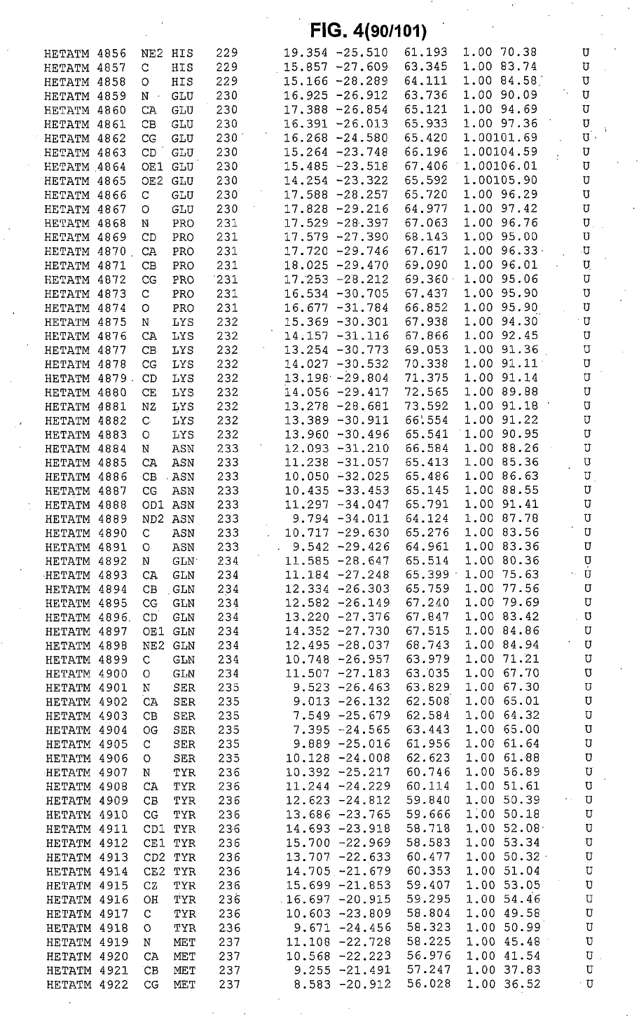

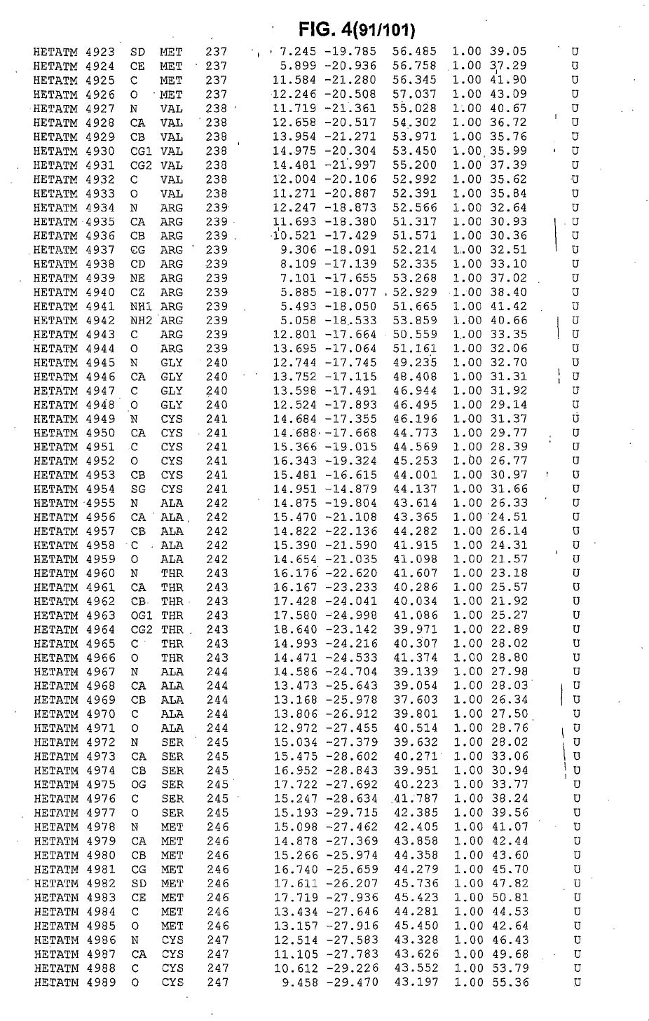

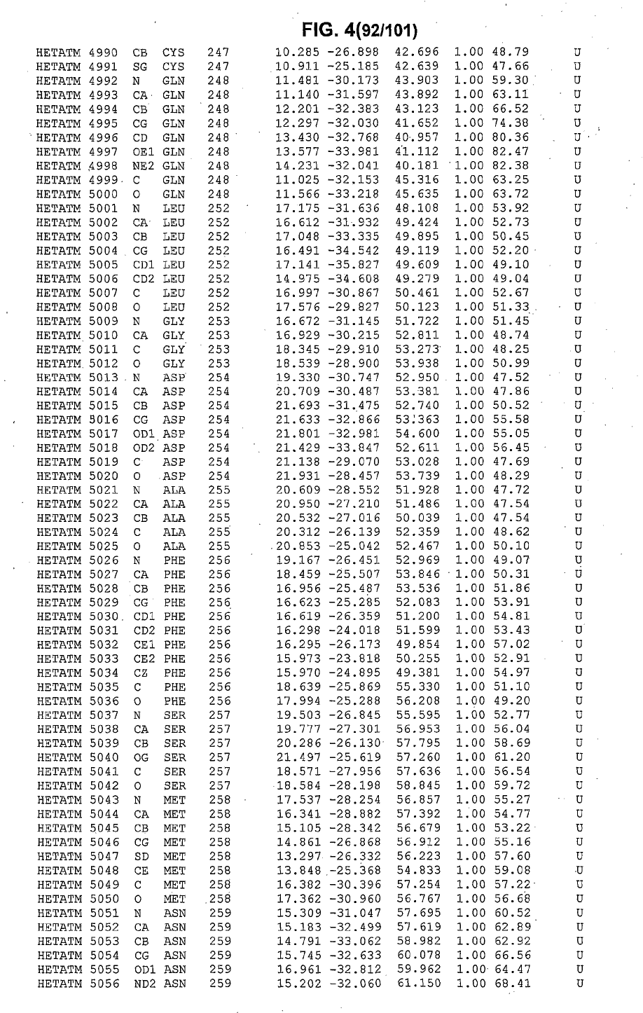

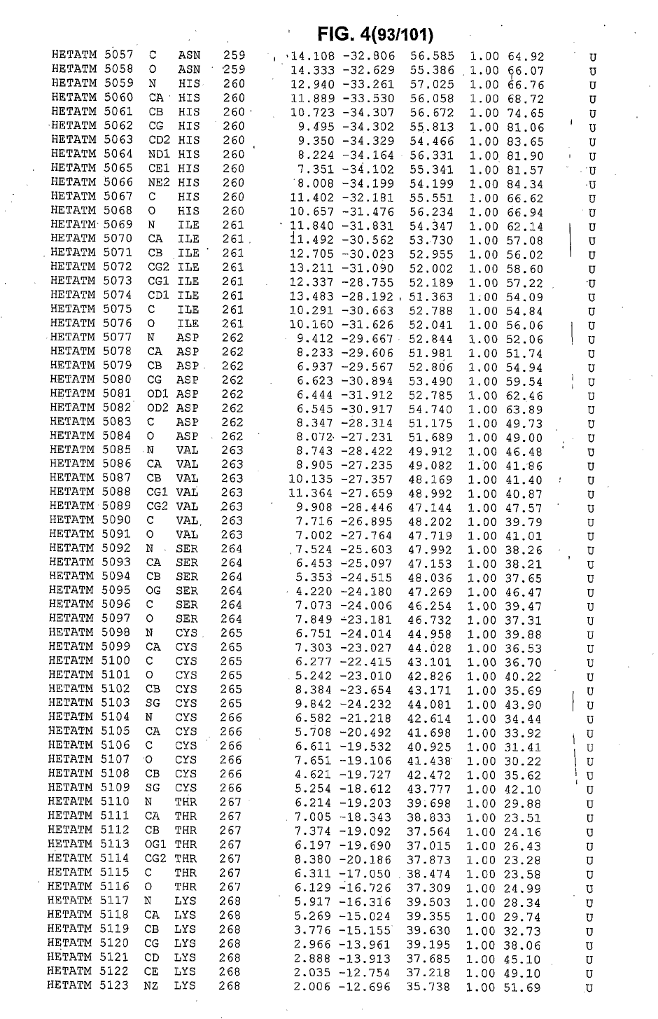

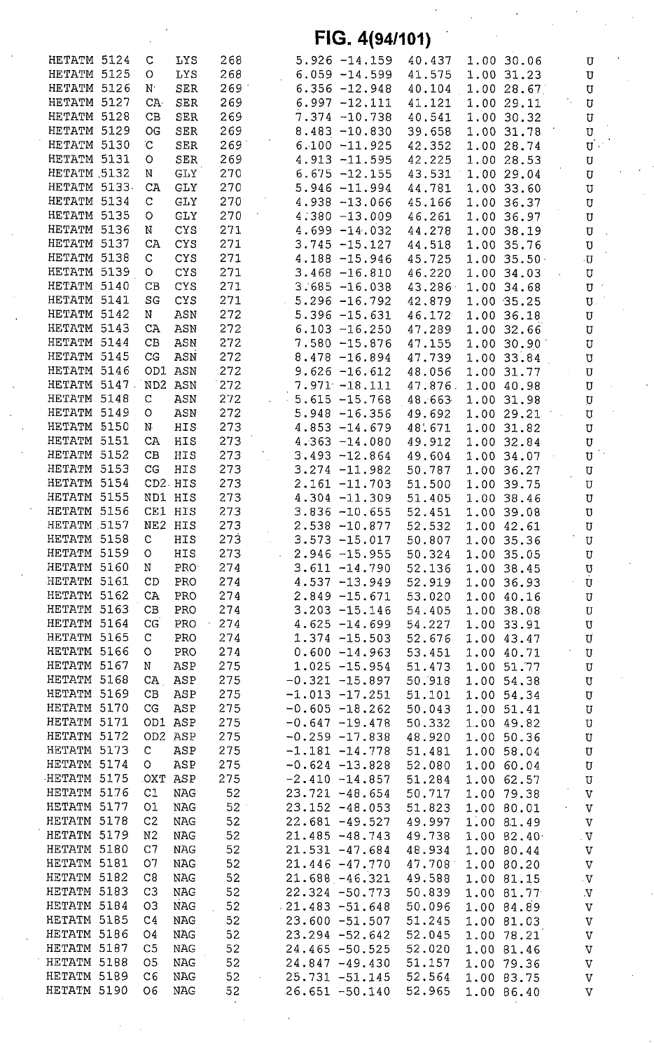

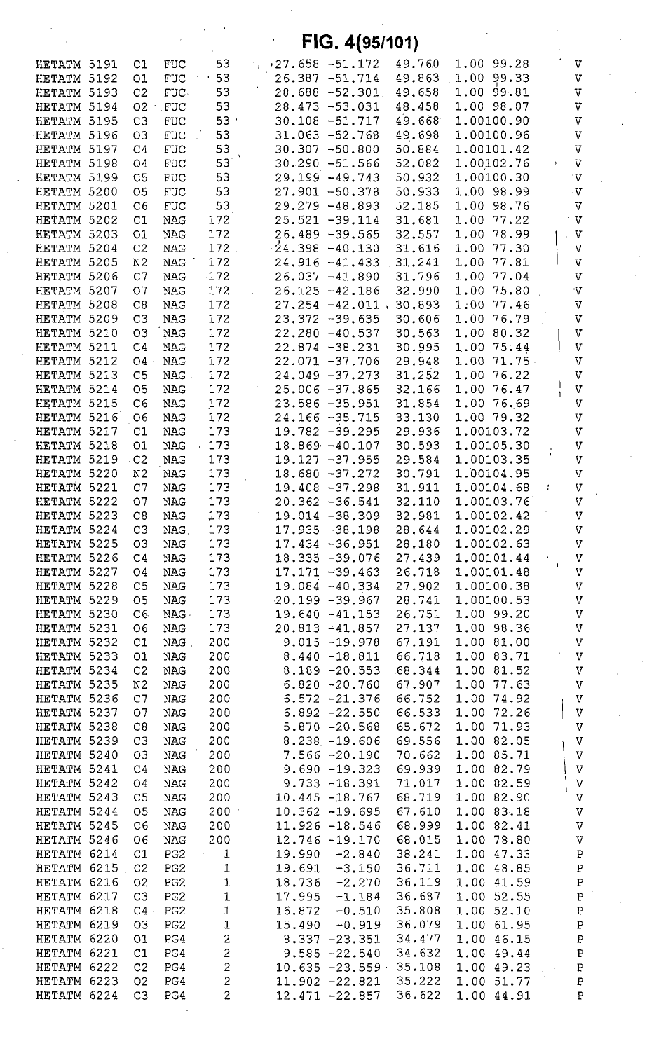

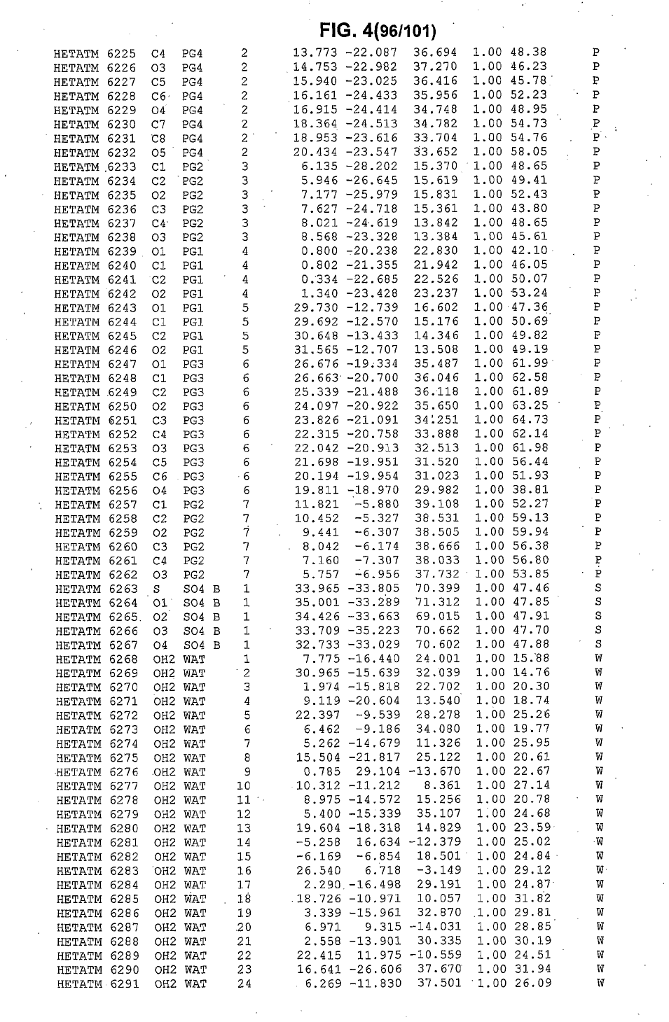

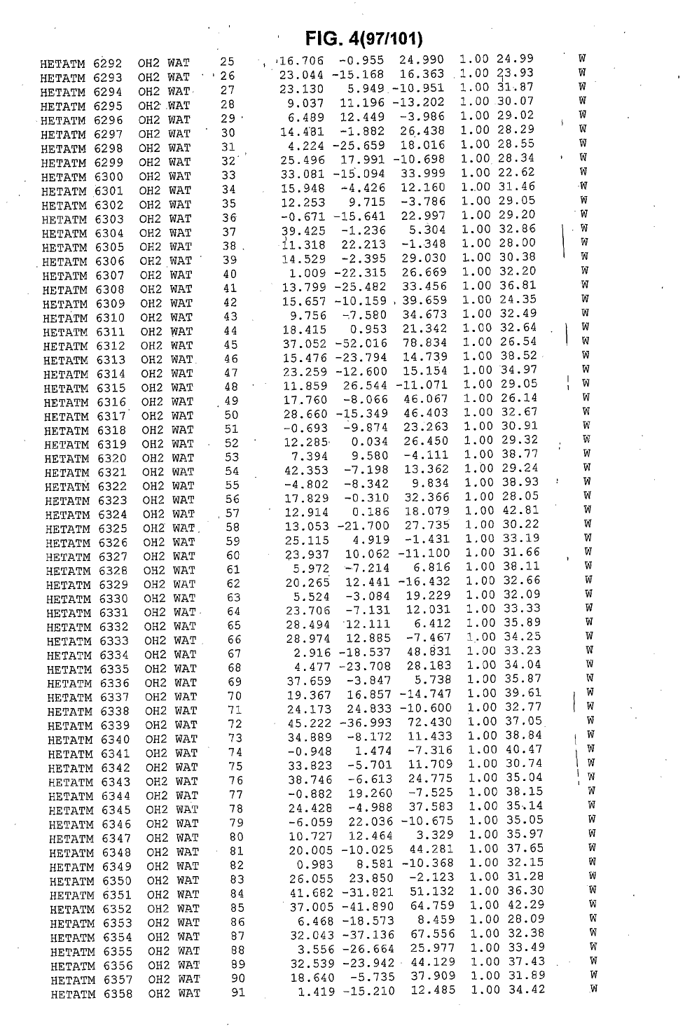

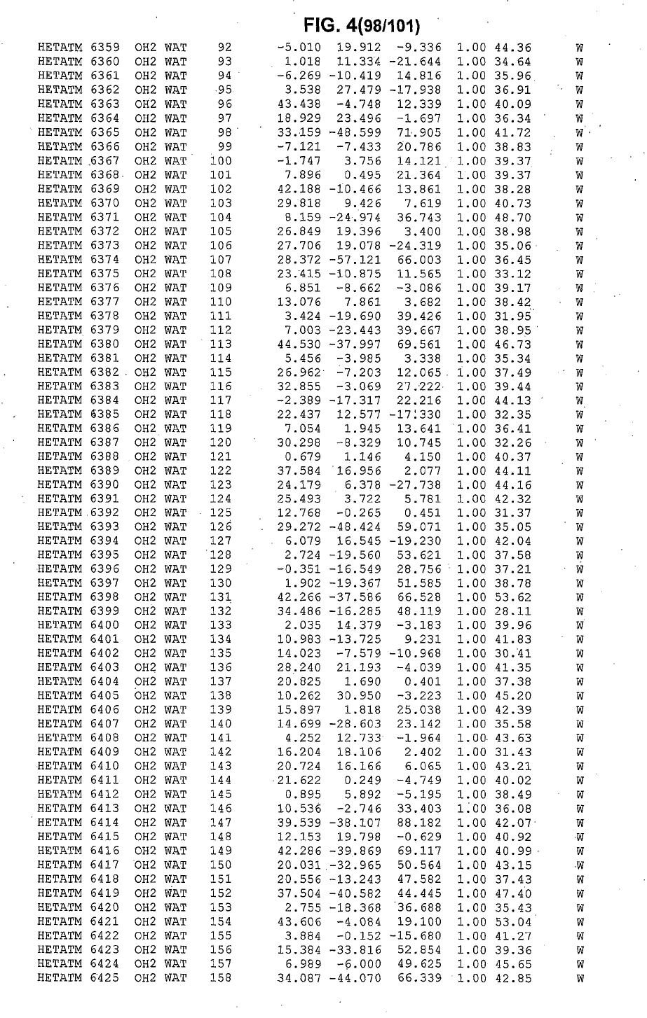

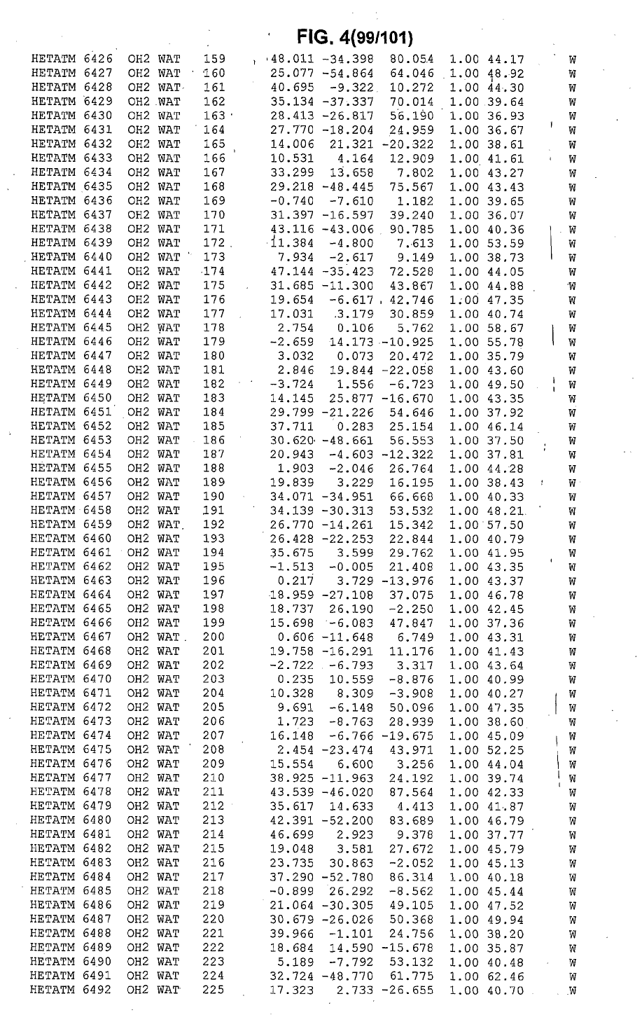

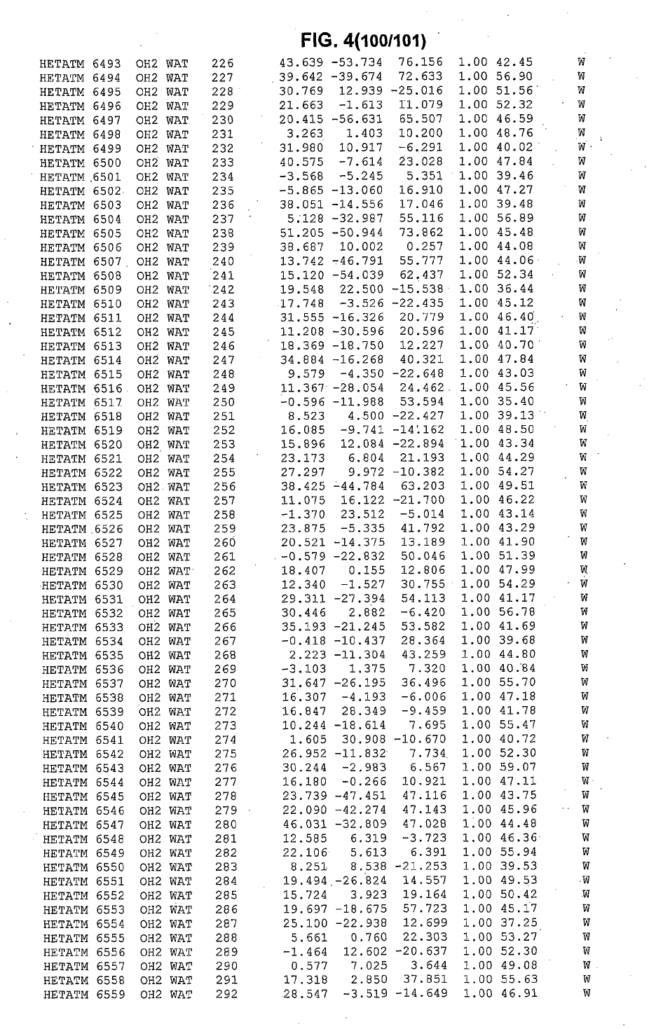

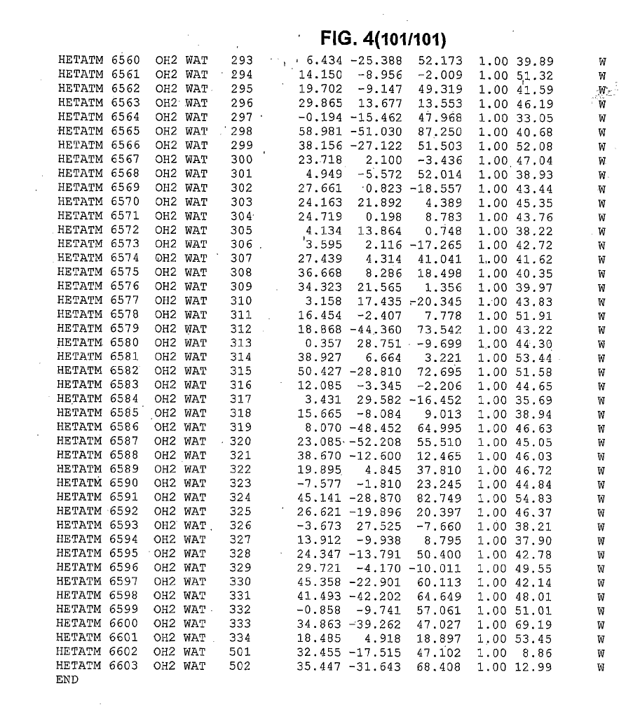

A complete data set of the ternary complex to 1.9 Å was collected using synchrotron radiation at the Advanced Photon Source (APS), Argonne National Laboratory. See FIG. 4. To solve the phase problem, the model of ATN-615 Fab fragment (Li, Y, X Shi, et al (2005) Prot Pep Lett.: in press) was positioned into the ternary complex crystal lattice by molecular replacement program, molrep (Vagin, A & A Teplyakov (1997) J Appl Cryst 30:1022-1025). Then, an iterative mask generation—solvent flattening—model building procedure was used to build the initial model of suPAR and uPA¹⁻¹⁴³. Briefly, a solvent mask that covered the ATN-615 Fab and extended along the Fab complementarity determining region (CDR) was manually constructed and used for the solvent flattening procedure by dm (Cowtan, K et al. (1998) Acta Crystallogr D Biol Crystallogr 54:487-93) on the phases derived from ATN-615 Fab model. Patterns of β strands were clearly visible in the antigen area, and a model with a poly-Ala backbone and some side chains was built by the arp-warp program (Lamzin, V S et al., (1993) Acta Crystallogr D Biol Crystallogr 49(Pt 1):129-47). The solvent mask was then modified and expanded to include more suPAR and uPA¹⁻¹⁴³ residues, and underwent the next cycle of model building.

The resulting model was refined using CNS and manual model fitting was carried out using the program O. The final model consists of 427 ATN residues (L and H chains), 122 uPA¹⁻¹⁴³ residues (A chain), 249 suPAR residues (U chain), 3 N-acetylglycosamines (V chain); 21 disulfide bonds; 1 glucose (V chain), 335 waters (W chain), 1 SO₄ (S chain) and 7 (poly)-ethylene glycol moieties (P chain).

### Example II

### Crystallization Strategy and Overall Structure

uPAR dimerizes in detergent-resistant lipid rafts on cell surfaces (Cunningham, O et al (2003) EMBO J 22:5994-6003). Recombinant suPAR from Drosophila S2 cells also tends to form oligomers in aqueous solution at concentrations required for protein crystallization (Llinas et al., supra). This posed great difficulties in trying to study uPAR's crystal structure. Previous studies by various of the present inventors and others showed that uPA could regulate uPAR oligomerization in vivo at the cellular level (Sidenius, N. et al (2002) J Biol Chem 277:27982-90) and dissociate suPAR oligomers in vitro (Shliom, O. et al (2000) J Biol Chem 275:24304-12), leading to the formation of crystallizable uPAR-uPA¹⁻¹⁴³ complexes at a 1:1 ratio. However, crystals obtained from this complex diffracted to only 3.1 Å (Huang et al., supra.)

The present inventors thus conceived of adding a uPAR binding partner, such as a uPAR-specific antibody or fragment (e.g., an Fab fragment) to constrain the 3D structure and thereby facilitate crystallization and improve the diffraction resolution of uPAR-uPA¹⁻¹⁴³ complexes. The mAb ATN-615, which had been raised against suPAR and which binds to suPAR at a domain that does not disrupt uPA-uPAR interactions was used in this capacity and is exemplified herein. Indeed, the ATN-615 Fab fragment facilitated suPAR-uPA¹⁻¹⁴³ crystallization, greatly improved the diffraction resolution of the crystals to 1.9 Å and provided phasing power to generate a discernible electron density map for suPAR and uPA¹⁻¹⁴³ model building.

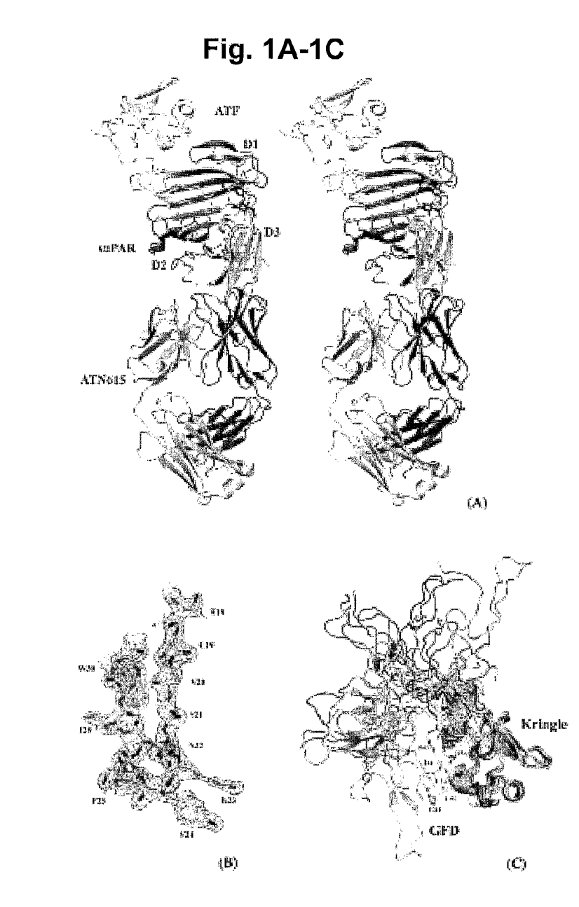

The electron density map disclosed herein shows that the majority of the structure in the uPAR-uPA¹⁻¹⁴³-Fab complexes was well-ordered. The receptor binding region of uPA is clearly defined in the electron density map (FIG. 1B). In the results disclosed herein, the loop that includes uPAR residues 35-37, 81-91, 130-139, 249-251 and uPA residues 1-10, 133-145 was omitted from the structure due to lack of electron density.

Because uPA¹⁻¹⁴³ binds mainly to the D1 domain of uPAR, and ATN-615 recognizes only the D3 domain at the other side, the three proteins in the ternary complex arrange into a linear and elongated complex with a length of 141 Å (FIG. 1A).

Data collection and refinement statistics are summarized in Table 1 below and the x-ray crystallographic details and coordinates appear in FIG. 4.

TABLE 1
Statistics on diffraction data and structure
refinement of suPAR-uPA¹⁻¹⁴³-ATN-615 complex.

| Data collection | suPAR-uPA¹⁻¹⁴³-ATN-615 |
|---|---|
| Space group | P21 |
| Unit cell | 51.792 Å, 86.805 Å, 124.690 Å, |
| | 90°, 94.54°, 90° |
| Resolution (Å) | 1.9 |
| Total measurements | 273,350 |
| Unique reflections | 86,852 |
| Completeness (%) | 94.5 (77.5) * |
| Average I/σ | 16.1 (1.97) * |
| Rmerge | 0.064 (0.338) * |

Structure Refinement

| | |
|---|---|
| R-factor | 0.237 |
| R-free | 0.274 |
| Resolution (Å) | 99-1.9 |

RMS deviation for

| | |
|---|---|
| Bond | 0.0069 Å |
| Angle | 1.47 |

* The numbers in parentheses are for the highest resolution shell.

### Example III

### Structure of uPA¹⁻¹⁴³ Bound to suPAR

The structure of the ternary complex as analyzed by the present X-ray analysis reveals both the uPA⁷⁻⁴³ (GFD) and the uPA⁵⁰⁻¹³⁵ (KrD) domains of uPA¹⁻¹⁴³ (cyan-colored molecule in FIG. 1A). The key feature in the uPA⁷⁻⁴³ domain are two short β-strands (uPA¹⁸⁻²² and uPA³⁰⁻³²) linked by an Ω-loop (uPA²³⁻²⁹), which regions serves a major receptor-binding determinant (Ploug, M (2003) Curr Pharm Des 9:1499-528). Of note is the observation that one of the disulfide bonds between uPA¹¹ and uPA¹⁹ was broken in this structure, possibly due to the disorder at the first 10 residues of uPA¹⁻¹⁴³. The KrD contains a two-stranded βsheet (residues uPA¹¹²⁻¹¹⁷ and uPA¹²⁰⁻¹²⁵), two short α-helices (uPA⁷⁸⁻⁸¹ and uPA⁹¹⁻⁹⁴) and three disulfide bonds.

In the unbound state, a structure obtained using NMR shows that the two domains of uPA¹⁻¹⁴³ and the KrD, exhibit a high degree of structural independence involving little or no inter-domain interaction (FIG. 1C) (Hansen, A P, A M Petros, et al (1994) Biochemistry 33:4847-64). However, when bound to its receptor (uPAR), uPA¹⁻¹⁴³ adopts a constrained conformation. The two domains of uPA¹⁻¹⁴³ pack more tightly leading to direct contacts between certain residues, e.g., L14 and H41 of uPA⁷⁻⁴³ GFD undergo hydrophobic interactions with L92 of uPA⁵⁰⁻¹²⁵ (FIG. 1C).

### Example IV

### Structure of Soluble uPA Receptor (suPAR) when Bound to ATF

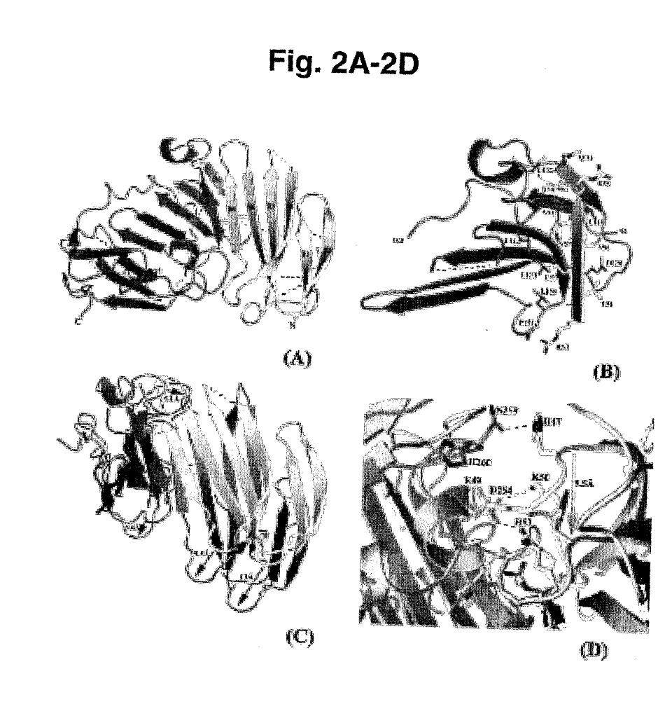

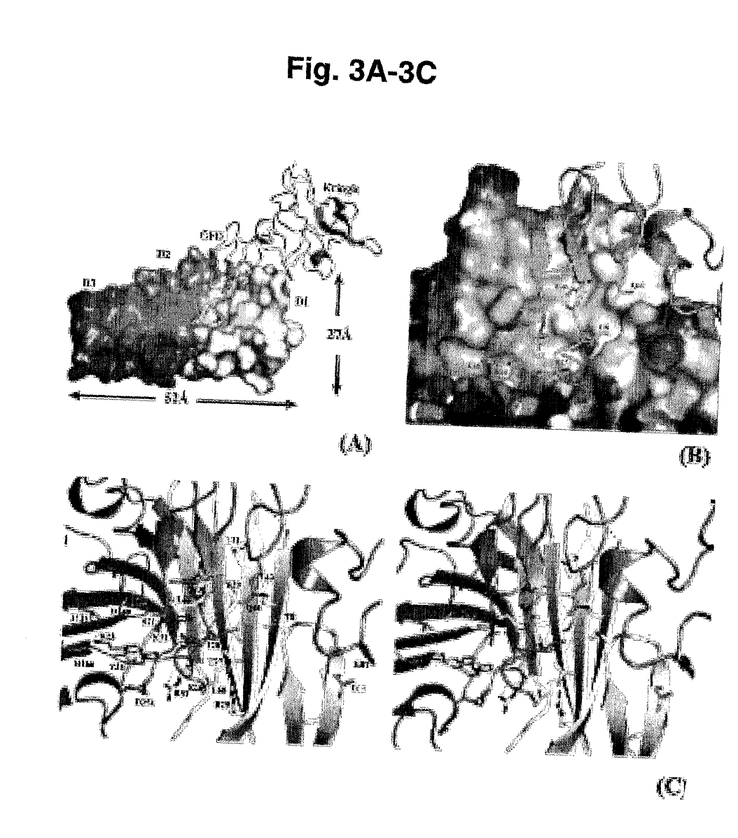

The structure of suPAR consists of 17 antiparallel β strands with three short α-helices, which are organized into three domains (FIG. 2A), consistent with what would be predicted from the sequence (Ploug et al., supra). The three domains of suPAR pack together to form the shape of a thick-walled teacup with a diameter of about 52 Å and a height of 27 Å (FIG. 3A). At the center of teacup, and surrounded by the three domains, is a cone shape cavity with a wide 25 Å opening and marked depth (14 Å) and large accessible surface for ligand binding (FIG. 3A). One characteristic of the cavity is a hydrophobic patch at its inner surface near the opening, formed mainly by D1 domain β strands β3 (L31 and V29), β4 (L40), β5 (L55) and β6 (L66) (FIG. 3B). This patch interacts with hydrophobic residues of uPA (see below).

The D1 domain comprises residues uPAR¹⁻⁸⁰ and a six-stranded antiparallel continued β-sheets (β1 to β6) constrained by three disulfide bonds. The β5 strand (uPAR⁵³⁻⁵⁸) is highly conserved across species and is essential for D1-D2 association. The D2 residues (uPAR⁹²⁻¹⁹¹) form a β sheet with six strands (β7 to β12), a short α-helix (α1, uPAR¹⁰⁴⁻¹⁰⁷) between β7 and β8, and four disulfide bonds. An interesting feature of D2 is that the β10 strand (uPAR¹⁴³⁻¹⁴⁹) twists about 60° at Gly146, so that the N-terminal half of this strand (uPAR¹⁴³⁻¹⁴⁵) is parallel with D2 β9, whereas the C-terminal half (uPAR¹⁴⁷⁻¹⁴⁹) lines up with the β5 of another domain (D1), suggesting a role in linking the domains (FIG. 2B). Also involved in D1-D2 association are β7 (94-99), β8 (uPAR¹¹¹⁻¹¹⁴), β11 (uPAR¹⁴³⁻¹⁴⁹) and a loop (uPAR¹⁰⁰⁻¹⁰⁴), resulting in six hydrogen bonds, a hydrophobic cluster on one side of the β5, several charge interactions on the other side of the β5, and an interface of 1188 Å²(FIG. 2B). The β11 and β12 strands of D2 are major determinants in D2-D3 association and form an even larger interface (1576 Å²) with D3. The D3 domain (residues 192 to 275) consists of a bundle of five β strands (β13 to β17) with two short α-helices (α2 and α3) linking β16 and β17. Four disulfide bonds are observed in this domain. Half of β13, β14, part of β15, β16, α1, α2, and a loop (uPAR²¹⁵⁻²¹⁹) of D3 are involved in the D2-D3 association. D1 and D3 also contact each other. The loop (uPAR²²⁶⁻²³⁷) and the α3 helix of D3 are involved in binding with the loop of the D1 domain (uPAR⁴⁷⁻⁵³), resulting in three hydrogen bonds between these two domains (H47-N259, K50-D254 and R53-D254, FIG. 2D) and an interface of 476 Å².

Structural superposition of the current uPAR structure with the suPAR in complex with a peptidyl inhibitor (Llinas et al., supra) shows that each domain of these two structures share similar folding with relatively small root-mean-squared deviation (rmsd) between two the structures, namely, 1.5 Å for D1 (77 Cα), 2.2 Å for D2 (89 Cα), 1.3 Å for D3 (81 Cα), 2.4 Å for D1D2 (166 Cα), 4 Å for D2D3 (170 Cα), respectively. However, the relative domain positions in the two suPAR structures show dramatic differences (FIG. 2C). When D2-D3 was superimposed between two suPAR structures, D1 showed a rotation of 20.5° and an rmsd of 9.5 Å (for 77 Cα) (FIG. 2C).

Compared with the presently described suPAR structure, the three loops (uPAR¹⁶⁻²³, uPAR⁴⁶⁻⁵³ and uPAR¹⁴⁹⁻¹⁵⁶) in the uPAR-inhibitor complex of Llinas et al., are shifted away from the center of binding cavity by about 13 Å, 8.5 Å and 5.6 Å, respectively, and six β strands in D1 domain shift by about 5-10 Å in order to enlarge the bottom of the binding cavity to accommodate the peptidyl inhibitor. Two loops (uPAR⁹⁹⁻¹⁰⁴) and (uPAR¹²⁸⁻¹⁴³) located at the opening of the uPAR cavity also show significant changes. Loop uPAR⁹⁹⁻¹⁰⁴ moves closer to D1 by 5.2 Å upon peptide (inhibitor) binding and this movement creates more space for uPA¹⁻¹⁴³ binding. Parts of the loop uPAR¹²⁸⁻¹⁴³ are disordered in both structures, but the stretch at both ends (residues 130-128 and 139-143) shows that this loop may play an important role in uPA¹⁻¹⁴³ binding. The domain associations D1-D2 and D1-D3 also undergo significant changes between two structures. In the suPAR/peptidyl complex, the D1-D3 interface decreases in area to 169 Å² and no hydrogen bond interaction were observed in this interface; the D1-D2 domain interface, especially β7, β8, β10 and the loop uPAR¹⁰⁰⁻¹⁰⁴ of D2 and β5 of D1 domain, also undergo significant shifts. These results highlight the dramatic conformational changes induced in uPAR by the binding of the peptidyl inhibitor and indicate that the domain-domain associations and the loops linking β-strands in uPAR are quite flexible. This suggests caution in designing uPAR inhibitors.

### Example V

### Binding Interface Between uPA and uPAR

uPA¹⁻¹⁴³ inserts into the cavity of uPAR (in a fashion that may be viewed as analogous to a teaspoon sitting in a teacup) forming a large interface of 1171 Å² (FIGS. 3B and 3C). This interface can be divided into three contact regions. The first is formed mainly by one stretch of residues in GFD (S21, N22, K23, Y24, the main chain of F25, and S26), which contacts primarily D2 of uPAR except for S26 of the GFD that interacts with a residue in D1 of uPAR. This region is buried deeply in the uPAR cavity and participates in hydrogen bonding and polar interactions between uPA and uPAR. Five of six hydrogen bonds in the uPAR-uPA¹⁻¹⁴³ interaction form in this region, e.g. the main chain O atom of S21 (uPA¹⁻¹⁴³) with the main chain N atom of D140 (uPAR); K23 (uPA¹⁻¹⁴³)-T127 (uPAR); S26 (uPA¹⁻¹⁴³)-E68 (uPAR). Y24 of uPA resides in a hydrophobic pocket and forms two hydrogen bonds with two domains of uPAR at residues R53 of D1 and residue H166 of D2, respectively) and polar interaction with D254 (in D3 of uPAR). This suggests residue Y24 is an important receptor binding determinant and is consistent with the results of biochemical studies (Ploug M et al (1995) Biochemistry 34:12524-34; Magdolen, V et al (1996) Eur J Biochem 237:743-51).

The second region of the uPAR-uPA interface is localized at the D1 hydrophobic patch (shown above), which interacts with three hydrophobic residues of uPA-F25, I128 and W30 (FIG. 3C). This region forms extensive hydrophobic interactions, and is thus a major contributor to the high affinity of uPA to uPAR.

The third region is localized to the edge of the teacup-shaped cavity and consists of a hydrogen bond (Q40 of uPA and T8 of uPAR D1) and van der Waals forces between uPAR and residues (Q40 and H87 from KrD) of uPA.

These results indicate that the uPAR D1 and D2 domains play important roles in the binding uPA. However, the D3 domain also undergoes direct interactions with uPA. Part of D3 (α3, uPAR²⁵³⁻²⁵⁵) contacts uPA by van der Waals interactions. D3 also forms a wall of the uPAR cavity and maintain the closeness of the cavity by interacting with D1 (FIG. 2D).

### Example VI

### Structural Basis for the Species Specificity Between Human and Mouse

uPA-uPAR binding is strongly species specific, as least between human and mouse (Ploug, M, S Ostergaard, et al (2001) Biochemistry 40:12157-68). Little or no binding occurs between human uPAR and murine uPA, and vice versa (Estreicher, A et al. (1989) J Biol Chem 264:1180-9.

Sequence alignment of uPAR residues involved in uPA binding show that most hydrophobic residues (4 of 5, that is, V29, L31, L40, L55, L66) and charged residues (5 out of 6, T8, R53, E68, T127, D140, H166) are conserved in all different species of uPAR. The only significant difference in murine uPAR compared to human uPAR is a change from L to E at residue 31. In human uPAR, L31 is a part of the hydrophobic patch (FIG. 3C) and plays an important role in binding to human uPA as observed in the present structure. This explains why murine uPAR cannot bind with human uPA.

On the uPA side, sequence alignment of the binding residues of uPA¹⁻¹⁴³ (or uPA¹⁻¹³⁵) in different species (Table 2) indicates significant variation of receptor binding residues (underscored in Table 2) in mouse when compared with human uPA. The W30R replacement in murine (vs. human) uPA is notable because human W30 is part of a hydrophobic cluster interacting with human uPAR's hydrophobic patch. Humanization of the murine uPA⁷⁻⁴³ by an R30W mutation (along with other mutations such as Y22N) resulted in high-affinity ligand for human uPAR (Quax, P H, J M Grimbergen, et al (1998) Arterioscler Thromb Vasc Biol 18:693-701.

This species specificity makes it difficult to test or screen for potential inhibitors of human uPAR-uPA interaction using mouse or rat uPAR (Ploug, 2003, supra; Behrendt, N (2004) Biol Chem 385:103-36). The structure defined herein provides a potential solution for this problem. Because residues involved in the first region of the uPAR-uPA interface are highly conserved among different species, antagonists targeting this region should inhibit both human and mouse uPAR-uPA interactions.

The structure of uPAR-uPA¹⁻¹⁴³ complex described herein provides a model that unifies and validates a large body of biochemical research on uPAR-uPA interactions (Ploug, 2003, supra; Behrendt, supra). Moreover, it provides a platform for rational design of inhibitors of uPAR-uPA interactions that may prevent, reverse or attenuate the pathophysiological consequences of these interactions, as in tumor metastasis.

TABLE 2

Sequence Alignment of uPA from Various Animal
Species

| | | | |
|---|---|---|---|
| 20 30 | 40 | 87 | SEQ ID NO |

| | | | | |
|---|---|---|---|---|
| HUMAN | GGTCVSNKYFSNIHWCN | Q | H | 1 |
| RAT | GGVCVSYKYFSSIRRCS | E | H | 2 |
| MOUSE | GGVCVSYKYFSRIRRCS | E | H | 3 |
| PIG | GGKCVSYKYFSNIQRCS | E | H | 4 |
| BOVINE | GGKCVTYKYFSNIQRCS | E | H | 5 |
| CHICK | GGTCITYRFFSQIKRCL | L | D | 6 |

Underscored residues interact with human uPAR.

The references cited herein are all incorporated by reference herein, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

1. A composition comprising a crystallized complex of uPA or a fragment thereof bound to a soluble uPAR and further bound to and constrained by a ligand which has an affinity of at least about 100 μM and which binds uPAR without disrupting uPA-uPAR binding interactions.

2. The composition-of claim 1, wherein said uPA is human uPA and said uPAR is human uPAR and said ligand is a uPAR-specific antibody or antigen-binding fragment thereof.

3. The composition-of claim 1, wherein said uPA is human uPA and said uPAR is human uPAR and said ligand is a uPA-specific antibody or an antigen-binding fragment thereof.

4. The composition of claim 2 wherein the ligand is a mAb designated ATN-615

5. The composition of claim 1, wherein a three-dimensional atomic structure of said complex is defined by a set of structural coordinates corresponding to the set of structure coordinates set forth in Table 1 and FIG. 4.

6. The composition of claim 1 wherein a three-dimensional atomic structure of said complex is defined by a set of structural coordinates having a root mean square deviation of not more than about 1.2 Å from the set of structure coordinates set forth in Table 1 and FIG. 4.

7. The composition of claim 6 wherein a three-dimensional atomic structure of said complex is defined by a set of structural coordinates having a root mean square deviation of not more than about 0.6 Å from the set of structure coordinates set forth in Table 1 and FIG. 4.

8. The composition of claim 7 wherein a three-dimensional atomic structure of said complex is defined by a set of structural coordinates having a root mean square deviation of not more than about 0.3 Å from the set of structure coordinates set forth in Table 1 and FIG. 4.

9. A computing platform for generating a 3D model of a uPA-uPAR complex further constrained by a uPAR ligand, which computing platform comprises:
(a) a data-storage device storing data comprising a set of structural coordinates defining the structure of at least a portion of a 3D crystal structure of the uPA-uPAR complex
(b) a data processing unit for generating the 3D model from the data stored in said data-storage device.

10. A computer generated model representing the conformationally constrained 3D structure of a uPA-uPAR complex to which is also bound a ligand for uPAR, the computer generated model having a 3D atomic structure defined by a set of x-ray crystallographic coordinates set out in Table 1 and FIG. 4.

11. A computer readable medium comprising, in a retrievable format, data that include a set of structure coordinates defining at least a portion of a 3D crystallographic structure of a crystallized uPA-uPAR complex that is conformationally constrained by being bound to a ligand for uPAR.

12. The computer readable medium of claim 11 wherein the ligand is an Fab fragment of mAb ATN-615

13. The computer readable medium of claim 11 wherein said structure coordinates defining at least a portion of a three-dimensional structure of the crystallized complex correspond to a set of coordinates set forth in Table 1 and FIG. 4.

14. The computer readable medium of claim 11 wherein said structural coordinates have a root mean square deviation of not more than 2 Å from a set of structural coordinates corresponding to a set of coordinates set forth in Table 1 and FIG. 4.

15. A method of crystallizing a ternary complex of uPA, soluble uPAR and a ligand for uPAR:
(a) forming a binary suPAR-uPA complex by incubating uPA with suPAR at a 1:1 molar ratio at room temperature in a first buffer, and purifying the complex;
(b) mixing the binary complex at a 1:1 molar ratio in a second buffer with a ligand that binds suPAR when the suPAR is bound to uPA, and purifying and concentrating a ternary 1:1:1 suPAR-uPA-ligand complex; and
(c) subjecting the ternary complex to microdialysis with a crystallization buffer, thereby crystallizing the ternary complex.


SEQUENCE LISTING (6 sequences)

SEQ ID NO: 1 - protein, 17 aa, Artificial Sequence
  Synthetic Peptide
GGTCVSNKYFSNIHWCN

SEQ ID NO: 2 - protein, 17 aa, Artificial Sequence
  Synthetic Peptide
GGVCVSYKYFSSIRRCS

SEQ ID NO: 3 - protein, 17 aa, Artificial Sequence
  Synthetic Peptide
GGVCVSYKYFSRIRRCS

SEQ ID NO: 4 - protein, 17 aa, Artificial Sequence
  Synthetic Peptide
GGKCVSYKYFSNIQRCS

SEQ ID NO: 5 - protein, 17 aa, Artificial Sequence
  Synthetic Peptide
GGKCVTYKYFSNIQRCS

SEQ ID NO: 6 - protein, 17 aa, Artificial Sequence
  Synthetic Peptide
GGTCITYRFFSQIKRCL